(19) United States
(12) Patent Application Publication
(10) Pub. No.: US 2025/0059612 A1
(43) Pub. Date: Feb. 20, 2025

(54) METHOD FOR SEPARATING GENOMIC DNA FOR AMPLIFICATION OF SHORT NUCLEIC ACID TARGETS

(71) Applicant: Becton, Dickinson and Company, Franklin Lakes, NJ (US)

(72) Inventors: Honghua Zhang, San Diego, CA (US); Daniel Jacob Koslover, San Diego, CA (US)

(21) Appl. No.: 18/721,621

(22) PCT Filed: Feb. 3, 2023
(86) PCT No.: PCT/US2023/061978

§ 371 Date: Jun. 18, 2024

Related U.S. Application Data
(60) Provisional application No. 63/307,085, filed on Feb. 5, 2022.

(51) Int. Cl. C12Q 1/689; C12Q 1/6806; C12Q 1/6844

(52) U.S. Cl. CPC C12Q 1/689; C12Q 1/6806; C12Q 1/6844

(57) ABSTRACT

Disclosed herein include methods, compositions, and kits for use in detecting a target nucleic acid sequence in a sample. Provided include acidic compositions for separating dsDNA. In some embodiments, the acidic compositions comprise a monovalent salt and/or a divalent salt, one or more surfactants, and an acidic agent. In some embodiments, the pH of the acidic composition is less than 4. Also provided herein are reagent compositions comprising a buffering agent and one or more amplification agents.

### RELATED APPLICATIONS

This present application is a National Stage application of PCT Application PCT/US2023/061978, filed on Feb. 3, 2023, which claims the benefit under 35 U.S.C. § 119 (e) of U.S. Provisional Patent Application Ser. No. 63/307,085, filed Feb. 5, 2022, the content of this related application is incorporated herein by reference in its entirety for all purposes.

### REFERENCE TO SEQUENCE LISTING

The present application is being filed along with a Sequence Listing in electronic format. The Sequence Listing is provided as a file entitled 68EB-317333-WO, created Feb. 2, 2023, which is 8.0 kilobytes in size. The information in the electronic format of the Sequence Listing is incorporated herein by reference in its entirety.

### BACKGROUND

### Field

The present disclosure relates generally to methods and compositions for amplification (e.g., isothermal amplification) of nucleic acids.

### Description of the Related Art

Nucleic acid-based diagnostics can be useful for rapid detection of infection, disease and/or genetic variations. For example, identification of bacterial or viral nucleic acid in a sample can be useful for diagnosing a particular type of infection. Other examples include identification of single nucleotide polymorphisms for disease management or forensics, and identification of genetic variations indicative of genetically modified food products. Often, nucleic acid-based diagnostic assays require amplification of a specific portion of nucleic acid in a sample. A common technique for nucleic acid amplification is the polymerase chain reaction (PCR). This technique typically requires a cycling of temperatures (i.e., thermocycling) to proceed through the steps of denaturation (e.g., separation of the strands in the double-stranded DNA (dsDNA) complex), annealing of oligonucleotide primers (short strands of complementary DNA sequences), and extension of the primer along a complementary target by a polymerase. Such thermocycling can be a time consuming process that generally requires specialized machinery. Thus, a need exists for quicker nucleic acid amplification methods that can be performed without thermocycling. Such methods can be useful, for example, for on-site testing and point-of-care diagnostics.

Most isothermal amplification methods require either a distinct heat denaturation step or the presence of a helicase, single-stranded DNA binding protein (SSB), or nicking enzyme for separation of genomic DNA strands. Since these requirements typically add either an extra step or considerable expense to the instrument and/or consumable, they are not as desirable as a purely chemical approach. A number of chemical methods, including alkaline treatment, formamide, glycerol, and DMSO, have previously been identified for separating strands of DNA for use in downstream applications. However, denaturants such as urea and formamide must be removed from solution prior to any amplification, which can be a time-consuming and costly step. Though the pH of an alkaline solution can be readily adjusted without chemical purification, its application is limited by the fact that magnesium ions are not readily soluble in aqueous solutions sufficiently alkaline to support separation of DNA strands. There is a need for compositions and methods of nucleic acid detection wherein chemical denaturation provides simple and rapid denaturation of DNA (without a need for sample separation, purification, or extraction steps) and is compatible with downstream amplification and detection steps.

### SUMMARY

Disclosed herein include methods for amplifying a target nucleic acid sequence in a sample. In some embodiments, the method comprises: (a) contacting a sample comprising biological entities with an acidic composition to generate an acidic mixture, wherein the acidic composition is capable of lysing biological entities to release sample nucleic acids comprised therein, wherein the sample nucleic acids comprise double-stranded DNA (dsDNA) suspected of comprising a target nucleic acid sequence, wherein the target nucleic acid sequence is no longer than 100 nucleotides in length, wherein the acidic composition comprises (i) a monovalent salt and/or a divalent salt, (ii) one or more surfactants, (iii), one or more chelating agents, and (iv) an acidic agent, and wherein the acidic composition has a pH of less than 4, thereby denaturing the dsDNA to generate single-stranded DNA (ssDNA); (b) contacting a reagent composition (e.g., a dried composition, a wet composition) comprising a buffering agent with the acidic mixture to generate a neutral mixture, wherein the neutral mixture comprises the ssDNA, wherein the reagent composition comprises one or more amplification reagents; and (c) amplifying the target nucleic acid sequence in the neutral mixture, thereby generating a nucleic acid amplification product. In some embodiments, the dsDNA suspected of comprising a target nucleic acid sequence is no longer than 100 base pairs in length. In some embodiments, one or more of the sample nucleic acids is no longer than 100 base pair in length.

Amplifying the target nucleic acid sequence can comprise generating the nucleic acid amplification product at detectable levels within about 20 minutes, about 15 minutes, about 10 minutes, or about 5 minutes. In some embodiments, the acidic composition has a pH of about 1 to about 6.9, for example a pH of about 1 to about 3.9 or a pH of about 2. In some embodiments, the neutral mixture has a pH of about 7 to about 9, for example a pH of about 8.8. The method can comprise: detecting the target nucleic acid sequence in the sample, wherein detecting the target nucleic acid sequence in the sample comprises: (d) detecting the nucleic acid amplification product, wherein the detecting is performed in less than about 20 minutes, less than about 15 minutes, less than about 10 minutes, or less than about 5 minutes, from the time the reagent composition is contacted with the acidic mixture.

Contacting the reagent composition (e.g., a dried composition, a wet composition) with the acidic mixture can comprise dissolving the reagent composition in the acidic mixture. The reagent composition can be a wet composition or a dried composition. In some embodiments, one or more amplification reagents comprise one or more of an enzyme having a hyperthermophile polymerase activity, a first primer, a second primer, and dNTPs. In some embodiments, the reagent composition is a wet composition (e.g., an aqueous composition, in a form suspended in liquid medium). In some embodiments, the reagent composition is lyophilized and/or heat-dried (e.g., a dried composition) and comprises one or more additives, wherein the one or more additives comprise: an amino acid; a sugar or sugar alcohol. In some embodiments, the sugar or sugar alcohol comprises sucrose, lactose, trehalose, dextran, erythritol, arabitol, xylitol, sorbitol, mannitol, or any combination thereof. In some embodiments, the one or more additives comprise a polymer. In some embodiments, the polymer comprises polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof.

In some embodiments, the dsDNA suspected of comprising the target nucleic acid sequence comprises a first strand and a second strand complementary to each other. In some embodiments, amplifying the target nucleic acid sequence comprises: amplifying the target nucleic acid sequence in an isothermal amplification condition, wherein the amplifying comprises contacting the ssDNA with: i) a first primer and a second primer, wherein the first primer is capable of hybridizing to a sequence of the first strand of the target nucleic acid sequence, and the second primer is capable of hybridizing to a sequence of the second strand of the target nucleic acid sequence; and ii) an enzyme having a hyperthermophile polymerase activity, thereby generating a nucleic acid amplification product, wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

The enzyme having a hyperthermophile polymerase activity can have an amino acid sequence that is at least about 90% identical or at least about 95% identical to the amino acid sequence of SEQ ID NO: 1 or a functional fragment thereof. In some embodiments, the enzyme having a hyperthermophile polymerase activity is a polymerase comprising the amino acid sequence of SEQ ID NO: 1. In some embodiments, the enzyme having a hyperthermophile polymerase activity has low or no exonuclease activity.

In some embodiments, the first primer and/or the second primer is about 8 to 16 bases long. In some embodiments, the first primer and/or the second primer comprises one or more of DNA bases, modified DNA bases, or a combination thereof. In some embodiments, the nucleic acid amplification product is about 20 to 40 bases long. In some embodiments, the spacer sequence comprises a portion of the target nucleic acid sequence. In some embodiments, the spacer sequence is 1 to 10 bases long. The dsDNA can comprise genomic DNA (gDNA), plasmid DNA, or both.

The sample can be, for example, a biological sample or an environmental sample. In some embodiments, the environmental sample is, or is obtained from, a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof. In some embodiments, the biological sample is, or is obtained from, a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof.

In some embodiments, the acidic agent comprises an organic acid, an inorganic acid, or both. In some embodiments, the acidic agent is selected from hydrochloric acid, glycine hydrochloride, acetic acid, citric acid, and phosphoric acid. In some embodiments, the acidic agent is present in the acidic composition at a concentration of less than about 20 mM. For example, the acidic agent can be present in the acidic composition at a concentration in the range of about 1 mM to about 100 mM, e.g., at a concentration of about 10 mM.

The monovalent salt and/or the divalent salt can comprise a sodium salt, a potassium salt, a calcium salt, a magnesium salt, or any combination thereof. In some embodiments, the monovalent salt is selected from ammonium sulfate, sodium fluoride, sodium chloride, sodium bromide, sodium iodide, potassium fluoride, potassium chloride, potassium bromide, and potassium iodide. In some embodiments, the divalent salt is selected from the group consisting of magnesium sulfate, calcium chloride, magnesium chloride, copper (II) chloride, zinc chloride, calcium oxide, magnesium oxide, barium oxide, sodium sulfate, calcium sulfate, copper (II) sulfate, potassium carbonate, and sodium carbonate. In some embodiments, the monovalent salt and/or the divalent salt is present in the acidic composition at a concentration in the range of about 1 mM to about 14 mM, for example a concentration of about 5 mM or about 4 mM.

The one or more surfactants can comprise one or more of a cationic surfactant, an anionic surfactant, a non-ionic surfactant, and an amphoteric surfactant. In some embodiments, the one or more surfactants is present in the acidic composition at a concentration in the range of about 0.01% to about 2% weight by volume (% w/v) of the acidic composition. In some embodiments, the one or more surfactants is present in the acidic composition at a concentration of about 0.1% weight by volume (% w/v) of the acidic composition. In some embodiments, the acidic composition further comprises a chelating agent. In some embodiments, the chelating agent is selected from ethylene diamine tetra-acetate (EDTA), ethylene glycol bis(amino ethyl) N,N′-tetra-acetate (EGTA), nitrilo-tri-acetate (NTA), and Tris. In some embodiments, the chelating agent is present in the acidic composition at a concentration in the range of about 0.1 mM to about 14 mM.

In some embodiments, contacting the sample with the acidic composition is performed at a temperature in the range of about 18° C. to about 99° C., for example at a temperature of about 78° C. In some embodiments, contacting the sample with the acidic composition is performed at a temperature in the range of about 18° C. to about 25° C. In some embodiments, contacting the sample with the acidic composition is performed for a period of about 5 seconds, about 10 seconds, about 20 seconds, about 30 seconds, about 40 seconds, about 50 seconds, about 1 minute, about 2 minutes, about 3 minutes, about 4 minutes, about 5 minutes, about 6 minutes, about 7 minutes, about 8 minutes, about 9 minutes, about 10 minutes, about 20 minutes, about 30 minutes, about 40 minutes, about 50 minutes, or about 60 minutes. In some embodiments, the buffering agent comprises MES, MOPS, MOPSO, Tris, HEPES, phosphate, acetate, citrate, succinate, ammonium buffers, or any combination thereof. In some embodiments, the neutral mixture has a pH in the range of about 7 to about 9, for example a pH of about 8.8.

In some embodiments, the target nucleic acid sequence comprises a length of no longer than about 20 nucleotides, no longer than about 30 nucleotides, no longer than about 40 nucleotides, no longer than about 50 nucleotides, no longer than about 60 nucleotides, or no longer than about 90 nucleotides. In some embodiments, the target nucleic acid sequence comprises a length of about 30 nucleotides. In some embodiments, the amplifying is performed in an isothermal amplification condition. In some embodiments, the isothermal amplification condition comprises a constant temperature of about 30° C. to about 72° C., for example a constant temperature of about 67° C. In some embodiments, the amplifying is performed for a period of about 5, 6, 7, 8, 9, 10, 20, 30, 40, 50, or 60 minutes, for example a period of about 15 minutes.

The amplifying can be performed in helicase-free, single-stranded binding protein-free, cleavage agent-free, and recombinase-free, isothermal amplification conditions. In some embodiments, step (d) further comprises determining the amount of the dsDNA that comprises the target nucleic acid sequence in the sample. In some embodiments, detecting the nucleic acid amplification product comprises use of a real-time detection method. In some embodiments, detecting the nucleic acid amplification product comprises contacting the nucleic acid amplification product with a signal-generating oligonucleotide capable of hybridizing to the nucleic acid amplification product, wherein the single-generating oligonucleotide comprises a fluorophore, a quencher, or both. In some embodiments, detecting the nucleic acid amplification product comprises detecting a fluorescent signal. In some embodiments, the fluorescent signal is from a molecular beacon. In some embodiments, the method is performed in a single reaction vessel. In some embodiments, the amplifying comprises multiplex amplification of two or more target nucleic acid sequences. In some embodiments, the detecting comprises multiplex detection of two or more nucleic acid amplification products derived from said two or more target nucleic acid sequences. In some embodiments, the two or more target nucleic acid sequences are specific to two or more different organisms.

In some embodiments, the amplifying does not comprise using any enzyme other than the enzyme having a hyperthermophile polymerase activity. In some embodiments, the amplifying comprises one or more of the following: Archaeal Polymerase Amplification (APA), loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA). In some embodiments, the amplifying does not comprise one or more of the following: Archaeal Polymerase Amplification (APA), LAMP, HDA, RPA, SDA, NASBA, TMA, NEAR, RCA, MDA, RAM, cHDA, SPIA, SMART, 3SR, GEAR and IMDA. In some embodiments, the amplifying does not comprise LAMP.

In some embodiments, the method does not comprise one or more of the following: (i) dilution of the acidic mixture; (ii) dilution of the neutral mixture; (iii) heat denaturation of the acidic mixture; (iv) sonication of the acidic mixture; (v) sonication of the neutral mixture; (vi) the addition of ribonuclease inhibitors to the acidic mixture; (vii) the addition of ribonuclease inhibitors to the neutral mixture; (viii) purification of the sample; (ix) purification of the sample nucleic acids; (x) purification of the nucleic acid amplification product; (xi) removal of the one or more surfactants from the acidic mixture or the neutral mixture; (xii) heating denaturing and/or enzymatic denaturing of the sample nucleic acids prior to and/or during amplification; (xiii) the addition of ribonuclease H to the acidic mixture or the neutral mixture; (xiv) contacting the sample, the acidic mixture, or the neutral mixture with a helicase, a single-stranded binding protein, a nicking enzyme, a restriction enzyme, or a recombinase; (xv) contacting the sample, the acidic mixture, or the neutral mixture with glycerol, formamide, or urea; (xvi) purifying the ssDNA from the acidic mixture or the neutral mixture; (xvii) a heat denaturing step; and (xviii) enriching, purifying and/or isolating the dsDNA.

In some embodiments, the acidic composition and/or the reagent composition comprises a reducing agent, a chelating agent, or both. In some embodiments, the acidic composition does not comprise a reducing agent, a chelating agent, or both. In some embodiments, the chelating agent is ethylene diamine tetra-acetate (EDTA), ethylene glycol bis(amino ethyl) N,N′-tetra-acetate (EGTA), nitrilo-tri-acetate (NTA), Tris, or any combination thereof. In some embodiments, the reducing agent is 2-mercaptoethanol, dithiothreitol (DTT), tris(2-carboxyethyl) phosphine (TCEP), dithioerythritol (DTE), reduced glutathione, cysteamine, tri-n-butylphosphine (TBP), dithioerythritol, tris(3-hydroxypropyl) phosphine (THPP), 2-mercaptoethylamine-HCl, dithiobutylamine (DTBA), cysteine, cysteine-thioglycolate, salts of sulfurous acid, thioglycolic acid, hydroxyethyldisulfide (HED), or any combination thereof. In some embodiments, the acidic composition does not comprise both an anionic surfactant and a cationic surfactant. In some embodiments, the acidic composition further comprises a tween surfactant, including but not limited to Tween 20, Tween 40, Tween 45, Tween 60, Tween 65, Tween 80, Tween 81 and Tween 85. In some embodiments, the tween surfactant comprises about 0.01% (w/v) of the acidic composition.

Disclosed herein include acidic compositions for lysing biological entities and denaturing dsDNA comprised therein. In some embodiments, the acidic composition comprises: a monovalent salt and/or a divalent salt; one or more surfactants; and an acidic agent, wherein the acidic agent is present in the acidic composition at a concentration of less than 100 mM, and wherein the acidic composition has a pH less than 4. The monovalent salt can be present in the acidic composition at a concentration of less than 30 mM. The divalent salt can be present in the acidic composition at a concentration of less than 15 mM.

In some embodiments, the acidic composition does not comprise a reducing agent, a chelating agent, or both. In some embodiments, the chelating agent is EDTA, EGTA, NTA, Tris, or any combination thereof. In some embodiments, the reducing agent is selected from 2-mercaptoethanol, DTT, TCEP, DTE, reduced glutathione, cysteamine, TBP, THPP, 2-mercaptoethylamine-HCl, DTBA, cysteine, cysteine-thioglycolate, salts of sulfurous acid, thioglycolic acid, HED, or any combination thereof. In some embodiments, the acidic composition does not comprise either an anionic surfactant or a cationic surfactant. In some embodiments, the acidic composition does not comprise glycerol, formamide, or urea.

In some embodiments, the acidic composition has a pH in the range of about 1 to about 3.9, for example a pH of about 2. In some embodiments, the acidic agent comprises an organic acid and/or an inorganic acid. In some embodiments, the acidic agent is selected from hydrochloric acid, glycine hydrochloride, acetic acid, citric acid, sulfuric acid, and phosphoric acid. In some embodiments, the acidic agent is present in the acidic composition at a concentration in the range of about 1 mM to about 100 mM, e.g., at a concentration of about 10 mM. In some embodiments, the monovalent salt and/or the divalent salt comprises a sodium salt, a potassium salt, a calcium salt, a magnesium salt, or any combination thereof. In some embodiments, the monovalent salt is selected from ammonium sulfate, ammonium chloride, sodium fluoride, sodium chloride, sodium bromide, sodium iodide, potassium fluoride, potassium chloride, potassium bromide, and potassium iodide. In some embodiments, the divalent salt is selected from magnesium sulfate, calcium chloride, magnesium chloride, copper (II) chloride, zinc chloride, calcium oxide, magnesium oxide, barium oxide, sodium sulfate, calcium sulfate, copper (II) sulfate, potassium carbonate, and sodium carbonate. In some embodiments, the monovalent salt and/or the divalent salt is present in the acidic composition at a concentration in the range of about 1 mM to about 14 mM, for example about 5 mM or about 4 mM. In some embodiments, the one or more surfactants comprises one or more of a cationic surfactant, an anionic surfactant, a non-ionic surfactant, and an amphoteric surfactant. In some embodiments, the one or more surfactants is present in the acidic composition at a concentration in the range of about 0.01% to about 2% weight by volume (% w/v) of the acidic composition, for example a concentration of about 0.1% weight by volume (% w/v) of the acidic composition. In some embodiments, the acidic composition further comprises a tween surfactant. In some embodiments, the tween surfactant is selected from Tween 20, Tween 40, Tween 45, Tween 60, Tween 65, Tween 80, Tween 81 and Tween 85. In some embodiments, the tween surfactant comprises about 0.01% to about 1% (w/v) of the acidic composition. In some embodiments, the acidic composition further comprises a chelating agent selected from ethylene diamine tetra-acetate (EDTA), ethylene glycol bis(amino ethyl) N,N′-tetra-acetate (EGTA), nitrilo-tri-acetate (NTA), Tris, and any combination thereof. In some embodiments, the chelating agent is present in the acidic composition at a concentration in the range of about 0.1 mM to about 14 mM.

Disclosed herein include kits for detecting a target nucleic acid sequence in a sample. In some embodiments, the kit comprises: (a) the acidic composition as provided herein, wherein the acidic composition is capable of lysing biological entities to release sample nucleic acids comprised therein, wherein the sample nucleic acids comprise dsDNA suspected of comprising a target nucleic acid sequence, wherein the target nucleic acid sequence is no longer than 100 nucleotides in length; and (b) a reagent composition comprising a buffering agent and one or more amplification reagents for amplifying the target nucleic acid sequence under isothermal amplification conditions, wherein said one or more amplification reagents comprise: (i) a first primer and a second primer, wherein the first primer is capable of hybridizing to a sequence of a first strand of the target nucleic acid sequence, and the second primer is capable to hybridizing to a sequence of a second strand of the target nucleic acid sequence; and (ii) an enzyme having a hyperthermophile polymerase activity capable of generating a nucleic acid amplification product. In some embodiments, the dsDNA suspected of comprising a target nucleic acid sequence is no longer than 100 base pairs in length. In some embodiments, one or more of the sample nucleic acids is no longer than 100 base pair in length.

In some embodiments, the buffering agent is selected from MES, MOPS, MOPSO, Tris, HEPES, phosphate, acetate, citrate, succinate, ammonium buffers, and any combination thereof. The kit can comprise: at least one component providing real-time detection activity for a nucleic acid amplification product. In some embodiments, the real-time detection activity is provided by a molecular beacon. In some embodiments, the enzyme having a hyperthermophile polymerase activity has an amino acid sequence that is at least about 90% identical or at least about 95% identical to the amino acid sequence of SEQ ID NO: 1 or a functional fragment thereof. In some embodiments, the enzyme having a hyperthermophile polymerase activity is a polymerase comprising the amino acid sequence of SEQ ID NO: 1. In some embodiments, the nucleic acid amplification product is about 20 to 40 bases long. In some embodiments, the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

In some embodiments, the first primer and/or the second primer is about 8 to 16 bases long. In some embodiments, the first primer and/or the second primer comprises one or more of DNA bases, modified DNA bases, or a combination thereof. In some embodiments, the reagent composition is lyophilized and/or heat-dried (e.g., a dried composition) and comprises one or more additives. The one or more additives can comprise: an amino acid; a sugar or sugar alcohol. The sugar or sugar alcohol can comprise sucrose, lactose, trehalose, dextran, erythritol, arabitol, xylitol, sorbitol, mannitol, or any combination thereof. In some embodiments, the one or more additives comprise a polymer. The polymer can comprise polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof.

In some embodiments, a mixture of the acidic composition and the reagent composition has a pH of about 7 to about 9, e.g., a pH of about 8.8. In some embodiments, a mixture of the sample, the acidic composition, and the reagent composition has a pH of about 7 to about 9, for example a pH of about 8.8. In some embodiments, the buffering agent comprises Tris. In some embodiments, a mixture of the sample, the acidic composition, and the reagent composition comprises Tris at a concentration in the range of about 30 mM Tris to about 50 mM Tris. In some embodiments, the kit comprises a sterile container housing the acidic composition and the reagent composition.

In the methods, compositions, kits, and systems disclosed herein, the biological entities can comprise one or more of prokaryotic cells, eukaryotic cells, viral particles, exosomes, protoplasts, and microvesicles. In some embodiments, the biological entities comprise a virus, a bacteria, a fungi, a protozoa, portions thereof, or any combination thereof. In some embodiments, the target nucleic acid sequence is a nucleic acid sequence of a virus, bacteria, fungi, or protozoa. In some embodiments, the sample nucleic acids are derived from a virus, bacteria, fungi, or protozoa. Non-limiting examples of viruses include Hepatitis B virus, Herpes Simplex, Herpesvirus 6, Herpesvirus 7, Epstein-Barr Virus, Cytomegalo-virus, Varicella-Zoster Virus, JC Virus, Parvovirus B19, Rotavirus, Human Adenovirus, and Genital Human Papillomavirus (HPV). Non-limiting examples of bacteria include Salmonella enterica, Streptococcus pyogenes, Clostridium difficile, Streptococcus agalactiae, Mycobacteria tuberculosis, Rickettsia rickettsii, Ehrlichia chaffeensis, Borrelia burgdorferi, Yersinia pestis, Treponema pallidum, Chlamydia trachomatis, Chlamydia pneumoniae, Mycoplasma pneumoniae, Mycoplasma sp., Legionella pneumophila, Legionella dumoffii, Mycoplasma fermentans, Ehrlichia sp., Haemophilus influenzae, Neisseria meningitidis, Neisseria gonorrhoeae, Streptococcus pneumonia, S. agalactiae, or Listeria monocytogenes. In some embodiments, the fungi is Coccidioides posadasii, Cryptococcus neoformans, Pneumocystis carinii, Histoplasma capsulatum, Blastomyces dermatitidis, Coccidioides immitis, or Trichophyton rubrum. In some embodiments, the protozoa is Trichomonas vaginalis, Trypanosoma cruzi, Leishmania sp., Plasmodium, Entamoeba histolytica, Babesia microti, Giardia lamblia, Cyclospora sp., and Eimeria sp.

### BRIEF DESCRIPTION OF THE DRAWINGS

FIG. 1A-FIG. 1B show non-limiting exemplary data related to the effect of a low pH solution (ARBT solution; FIG. 1B) on dissociation of gDNA for Archaeal Polymerase Amplification (APA) as compared to a non-acidic control (TE solution; FIG. 1A). Legend indicates number of target sequence copies in the sample. NTC, no target control. LOD, limit of detection.

FIG. 2 shows non-limiting exemplary data related to the effect of pre-incubation temperature on gDNA dissociation. Legend indicates number of target sequence copies in the sample and pre-incubation temperature. NTC, no target control. LOD, limit of detection.

FIG. 3A-FIG. 3B show non-limiting exemplary data related to the effect of elution buffer pH on N. gonorrhoeae (Ng) gDNA detection (in a wet reaction), with acidic elution buffer (FIG. 3B) compared to non-acidic control elution buffer (FIG. 3A). Legend indicates number of target sequence copies in the sample. NTC, no target control. LOD, limit of detection.

FIG. 4A-FIG. 4B show non-limiting exemplary data related to the effect of elution buffer pH (FIG. 4B) on N. gonorrhoeae (Ng) gDNA detection (in a reaction using dried lyophilized pellets), with acidic elution buffer (FIG. 4B) compared to non-acidic control elution buffer (FIG. 4A). Legend indicates number of target sequence copies in the sample. NTC, no target control. LOD, limit of detection.

FIG. 5A-FIG. 5B show non-limiting exemplary data related to the effect of temperature and pH on N. gonorrhoeae (Ng) gDNA stability, with incubations performed at room temperature (RT; FIG. 5A) or at 78° C. (FIG. 5B) before mixing with amplification components. Legend indicates incubation in acidic (ARBT) or non-acidic (TE) conditions with indicated incubation times and temperatures.

FIG. 6A-FIG. 6B show non-limiting exemplary data related to the effect of temperature and pH on N. gonorrhoeae (Ng) gDNA stability in 10% urine matrix, with incubations performed at room temperature (RT; FIG. 6B) or at 78° C. (FIG. 6A) before mixing with amplification components. Legend indicates incubation in acidic (ARBT) or non-acidic (TE) conditions with indicated incubation times and temperatures. NTC, no target control.

FIG. 7 shows non-limiting exemplary data related to the effect of temperature and pH on N. gonorrhoeae (Ng) gDNA stability (in a dry reaction format). Legend indicates incubation time and temperatures. NTC, no target control.

FIG. 8 shows non-limiting exemplary melting curves of λ DNA as a function of pH (adapted from Biophys J. 2001 February; 80 (2): 874-881. Effect of pH on the overstretching transition of double-stranded DNA: evidence of force-induced DNA melting).

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include methods, compositions, and kits for lysing biological entities and separating double-stranded polynucleotides comprised therein for amplifying and detecting one or more target nucleotides sequences.

Disclosed herein include methods for amplifying a target nucleic acid sequence in a sample. In some embodiments, the method comprises: (a) contacting a sample comprising biological entities with an acidic composition to generate an acidic mixture, wherein the acidic composition is capable of lysing biological entities to release sample nucleic acids comprised therein, wherein the sample nucleic acids comprise dsDNA suspected of comprising a target nucleic acid sequence, wherein the target nucleic acid sequence is no longer than 100 nucleotides in length, wherein the acidic composition comprises (i) a monovalent salt and/or a divalent salt, (ii) one or more surfactants, and (iii) an acidic agent, and wherein the acidic composition has a pH of less than 4, thereby denaturing the dsDNA to generate single-stranded DNA (ssDNA); (b) contacting a reagent composition (e.g., a dried composition, a wet composition) comprising a buffering agent with the acidic mixture to generate a neutral mixture, wherein the neutral mixture comprises the ssDNA, wherein the reagent composition comprises one or more amplification reagents; and (c) amplifying the target nucleic acid sequence in the neutral mixture, thereby generating a nucleic acid amplification product.

Disclosed herein include acidic compositions for lysing biological entities and denaturing dsDNA comprised therein. In some embodiments, the acidic composition comprises: a monovalent salt and/or a divalent salt; one or more surfactants; and an acidic agent, wherein the acidic agent is present in the acidic composition at a concentration of less than 100 mM, and wherein the acidic composition has a pH less than 4. The monovalent salt can be present in the acidic composition at a concentration of less than 30 mM. The divalent salt can be present in the acidic composition at a concentration of less than 15 mM.

Disclosed herein include kits for detecting a target nucleic acid sequence in a sample. In some embodiments, the kit comprises: (a) the acidic composition as provided herein, wherein the acidic composition is capable of lysing biological entities to release sample nucleic acids comprised therein, wherein the sample nucleic acids comprise dsDNA suspected of comprising a target nucleic acid sequence, wherein the target nucleic acid sequence is no longer than 100 nucleotides in length; and (b) a reagent composition (e.g., a dried composition, a wet composition) comprising a buffering agent and one or more amplification reagents for amplifying the target nucleic acid sequence under isothermal amplification conditions, wherein said one or more amplification reagents comprise: (i) a first primer and a second primer, wherein the first primer is capable of hybridizing to a sequence of a first strand of the target nucleic acid sequence, and the second primer is capable to hybridizing to a sequence of a second strand of the target nucleic acid sequence; and (ii) an enzyme having a hyperthermophile polymerase activity capable of generating a nucleic acid amplification product. In some embodiments, the dsDNA suspected of comprising a target nucleic acid sequence is no longer than 100 base pairs in length.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the term “acid” shall be given its ordinary meaning, and, in some embodiments, an “acid” can be either an Arrhenius acid, a Bronsted-Lowry acid, or a Lewis acid. The Arrhenius acids are substances or fluids which increase the concentration of hydronium ions (H₃O⁺) in solution. The Brønsted-Lowry acid is a substance which can act as a proton donor. Lewis acids are electron-pair acceptors. As used herein, the term “base” refers to either a substance that can accept hydrogen cations (protons) or more generally, donate a pair of valence electrons. A soluble base is referred to as an alkali if it contains and releases hydroxide ions (OH⁻) quantitatively. The Brønsted-Lowry theory defines bases as proton (hydrogen ion) acceptors, while the more general Lewis theory defines bases as electron pair donors, allowing other Lewis acids than protons to be included.

As used herein, the term “buffer” shall be given its ordinary meaning, and shall also refer to aqueous solutions or compositions that resist changes in pH when acids or bases are added to the solution or composition. This resistance to pH change is due to the buffering properties of such solutions. Thus, solutions or compositions exhibiting buffering activity are referred to as buffers or buffer solutions. Buffers generally do not have an unlimited ability to maintain the pH of a solution or composition. Rather, they are typically able to maintain the pH within certain ranges, for example between pH 7 and pH 9. Typically, buffers are able to maintain the pH within one log above and below their pKa. Buffers and buffer solutions are typically made from buffer salts or from non-ionic buffer components like TRIS and HEPES. Non-limiting examples of buffers include MES, MOPS, MOPSO, Tris, HEPES, phosphate, acetate, citrate, succinate, and ammonium buffers, as well as combinations of these.

Provided herein are methods and compositions for amplifying nucleic acid. Traditional nucleic acid amplification methods typically require a thermocycling process, nucleic acid denaturation, proteins (e.g., enzymes) that promote strand unwinding, strand separation and/or strand exchange (e.g., helicases, recombinases), and/or endonuclease agents (e.g., restriction enzymes, nicking enzymes), and often require a minimum reaction time of 20 to 30 minutes. The nucleic acid amplification methods provided herein can be performed without thermocycling, without thermal denaturation and/or enzymatic denaturation of sample nucleic acids, without added proteins (e.g., enzymes) to promote strand unwinding, strand separation and/or strand exchange, without endonuclease agents, and within a reaction time of about 10-15 minutes.

### Chemical Methods for the Separation of Genomic Strands of DNA for Amplification of Short Nucleic Acid Targets

Provided herein include methods and compositions for direct pathogen lysis in clinical samples to enable isothermal amplification using an archaeal polymerase and real-time detection of nucleic acids. Rapid point of care (POC) diagnostics can be developed that do not require sample purification. Viral particles, bacterial cells, or other pathogens still need be lysed so that their DNA and/or RNA can be released from the cell and can be available for an amplification reaction. Conventional chemical lysis methods (e.g., employing strong bases, ionic detergents, and chaotropic agents) are incompatible with enzyme function, as these agents will also inactivate any DNA polymerase or other enzymes. Therefore, there is significant value in an effective chemical lysis method that is compatible with enzyme function and does not require any nucleic acid purification steps to remove the lysis reagents.

Provided herein include fast and simple methods for separating complementary strands of dsDNA (e.g., genomic DNA (gDNA)) for subsequent targeting of short sequence stretches in, e.g., amplification reactions. In some embodiments, the separation of strands is accomplished through the use of an acidic agent (e.g., an inorganic acid) such as HCl, and can be performed concurrently with sample lysis. Without being bound by any particular theory, once lysis releases the dsDNA, protonation and spontaneous loss of hydrogen bonding between complementary bases enables the dissociation of DNA strands in the lysis solution (e.g., acidic composition). In some embodiments, the denatured DNA sample is mixed with reaction components (e.g., reagent composition) for subsequent amplification at a constant temperature without any separation or purification steps. In some embodiments, amplification reaction components includes an amplification buffer (e.g., reagent composition) with sufficient capacity to neutralize the acid and raise the pH to that required for rapid amplification. In some embodiments, the amplification reaction contains primers complementary to the target gDNA at high concentration to ensure the formation of gDNA-primer complexes occurs more rapidly and frequently than the spontaneous reformation of the original double-stranded gDNA. In some embodiments, amplification can be initiated by primers extending on the gDNA targets through the incorporation of nucleotides by one or more DNA polymerases.

Disclosed herein includes the surprising finding that exposing genomic DNA to a low pH solution as an initial step in Archaeal Polymerase Amplification does not adversely affect amplification as is commonly believed. Instead, as shown in the Examples, this approach can increase the speed of isothermal amplification as well as yield improvement in sensitivity of genomic DNA detection up to 100 fold. The methods, compositions, and kits provided herein can advantageously provide a point-of-care molecular diagnostic for pathogens (e.g., DNA pathogens) in clinical samples. Following pathogen lysis, genomic DNA strands are separated with, in some embodiments, no need for a separate thermal or enzymatic step.

There are provided, in some embodiments, sample preparation methods employing acid dissociation for Archaeal Polymerase Amplification (APA). Without being bound by any particular theory, in some embodiments the acidic conditions function to denature or dissociate genomic DNA materials to facilitate isothermal APA amplification. In some embodiments, the presence of an acidic agent in the acidic compositions (e.g., resuspension buffer, elution buffer, lysis buffer) disclosed herein can also improve biological entity lysis efficiency (e.g., cell lysis efficiency). The disclosed acid dissociation approach can be employed for denaturation of DNA in a variety in nucleic acid detection methods, such as isothermal amplification methods including LAMP, NEAR, RCA, MDA, and RPA to improve initiation on genomic DNA for improvement of amplification efficiency. The methods, compositions, and kits provided herein are not limited to isothermal amplification methods and they can be applied to applications requiring single stranded DNA or dissociation of DNA such as forensic analysis.

Currently available methods do not employ acidic conditions in the sample preparation process for DNA dissociation or pathogen lysis for direct DNA amplification. Most isothermal amplification methods require a prior heat denaturation step, chemical denaturation, or enzymes that unwind genomic DNA. They are poorly suited for a point of care setting. Accordingly, there is a need for a simple and rapid denaturation method, which is addressed by the compositions and methods provided herein.

Although dsDNA is known to spontaneously separate in acidic solutions, their use is widely considered to be incompatible with subsequent amplification of DNA due to the detrimental effects arising from hydrolytic depurination. Depurination, one of the discoveries for which Thomas Lindahl was awarded a Nobel prize in chemistry in 2015, is a naturally occurring process under physiological conditions. It is a chemical reaction of purine deoxyribonucleosides in which the β-N-glycosidic bond between a purine base guanine or adenine and deoxyribose moiety is hydrolytically cleaved. The cleavage of the base-sugar bond results in the loss of genetic information and formation of an abasic site in DNA, and an alteration of the DNA structure. DNA damage from depurination has been shown to be problematic for amplification of PCR products, as a single DNA lesion within the template is sufficient to stall the PCR enzyme (e.g., DNA polymerase).

The methods, compositions, and kits disclosed herein resolve the above-mentioned needs and overcome the challenges presented by acid-catalyzed DNA damage by pairing the use of acidic separation with the amplification of short DNA sequences (e.g., about 30 nucleotides in length). In some embodiments, an acidic agent is included in the elution/lysis solution which dissociates genomic DNA upon lysis of biological entities (e.g., lysis of pathogen cell walls). The inclusion of a high-capacity buffer provided herein in the amplification step can ensure that the acid is neutralized and an optimal pH condition is maintained. The acidic composition (e.g., resuspension buffer, elution solution, lysis solution) also contains, in some embodiments, low concentrations of monovalent salt and/or divalent magnesium, which have been reported to suppress depurination, possibly, and without being bound by any particular theory, by neutralizing negative charges on phosphate groups on the DNA backbones. Unlike alkaline treatment in which magnesium is insoluble at high pH, magnesium is readily solubilized at acidic pH.

Disclosed herein include methods for amplifying a target nucleic acid sequence in a sample. In some embodiments, the method comprises: (a) contacting a sample comprising biological entities with an acidic composition to generate an acidic mixture, wherein the acidic composition is capable of lysing biological entities to release sample nucleic acids comprised therein, wherein the sample nucleic acids comprise dsDNA suspected of comprising a target nucleic acid sequence, wherein the target nucleic acid sequence is no longer than 100 nucleotides in length, wherein the acidic composition comprises (i) a monovalent salt and/or a divalent salt, (ii) one or more surfactants, and (iii) an acidic agent, and wherein the acidic composition has a pH of less than 4, thereby denaturing the dsDNA to generate single-stranded DNA (ssDNA); (b) contacting a reagent composition (e.g., a dried composition, a wet composition) comprising a buffering agent with the acidic mixture to generate a neutral mixture, wherein the neutral mixture comprises the ssDNA, wherein the reagent composition comprises one or more amplification reagents; and (c) amplifying a target nucleic acid sequence in the neutral mixture, thereby generating a nucleic acid amplification product. In some embodiments, the dsDNA suspected of comprising a target nucleic acid sequence is no longer than 100 base pairs in length. In some embodiments, one or more of the sample nucleic acids is no longer than 100 base pair in length. In some embodiments, the dsDNA suspected of comprising a target nucleic acid sequence is longer than 100 base pairs in length. In some embodiments, one or more of the sample nucleic acids is longer than 100 base pair in length. The sample nucleic acids and/or the dsDNA suspected of comprising a target nucleic acid sequence can be about 100 nt, 500 nt, 1 kb, 5 kb, 10 kb, 50 kb, 100 kb, 200 kb, 300 kb, 400 kb, 500 kb, 600 kb, 700 kb, 800 kb, 900 kb, 1 Mb, 2 Mb, 3 Mb, 4 Mb, 5 Mb, 6 Mb, 7 Mb, 8 Mb, 9 Mb, 10 Mb, 20 Mb, 30 Mb, 40 Mb, 50 Mb, 60 Mb, 70 Mb, 80 Mb, 90 Mb, or 100 Mb, or a number or a range between any two of these values, nucleotides in length. The sample nucleic acids and/or the dsDNA suspected of comprising a target nucleic acid sequence can be fully denatured upon the contacting with the acidic composition. In some embodiments, a portion of a sample nucleic acid molecule and/or dsDNA molecule suspected of comprising a target nucleic acid sequence is denatured (e.g., local denaturing) upon the contacting with the acidic composition. In some embodiments, a portion of a sample nucleic acid molecule and/or dsDNA molecule suspected of comprising a target nucleic acid sequence is denatured (e.g., local denaturing) in the acidic mixture and a portion of said molecule remains in a double-stranded form.

Amplifying the target nucleic acid sequence can comprise generating the nucleic acid amplification product at detectable levels within about 20 minutes, 15 minutes, or about 10 minutes (e.g., within about 20, 19, 18, 17, 16, 15, 14, 13, 12, 11, 10, 9, 8, 7, 6, 5, 4, 3, 2, 1, or a number or a range between any two of these values, minutes). In some embodiments, steps (b) and (c) are performed concurrently (e.g., the amplification begins once the contacting of the reagent composition and the acidic mixture has occurred). In some embodiments, the reagent composition comprises two or more dried compositions (comprising the same or different components) or two or more wet compositions (comprising the same or different components). In some embodiments, the acidic composition comprises two or more acidic agents (comprising the same or different components).

The acidic composition can have a pH of about 1 to about 6.9 (e.g., about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, or a number or a range between any two of these values). In some embodiments, the acidic composition has a pH of about 1 to about 3.9, for example a pH of about 2 or a pH of about 2, about 2.1, about 2.2, about 2.3, about 2.4, and about 2.5. In some embodiments, the acidic composition has a pH of about 2.2. In some embodiments, the neutral mixture has a pH of about 7 to about 9 (e.g., about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, or a number or a range between any two of these values). In some embodiments, the neutral mixture has a pH of about 8.8.

The method can comprise: detecting the target nucleic acid sequence in the sample, wherein detecting the target nucleic acid sequence in the sample comprises: (d) detecting the nucleic acid amplification product, wherein the detecting is performed in less than about 20 minutes, less than about 15 minutes, or less than about 10 minutes (e.g., about 20, 19, 18, 17, 16, 15, 14, 13, 12, 11, 10, 9, 8, 7, 6, 5, 4, 3, 2, 1, or a number or a range between any two of these values, minutes), from the time the reagent composition is contacted with the acidic mixture.

Contacting the reagent composition (e.g., a dried composition, a wet composition) with the acidic mixture can comprise dissolving the reagent composition in the acidic mixture. The one or more amplification reagents can comprise one or more of an enzyme having a hyperthermophile polymerase activity, a first primer, a second primer, and dNTPs.

In some embodiments, the reagent composition (e.g., a dried composition) is lyophilized and/or heat-dried and comprises one or more additives, wherein the one or more additives comprise: an amino acid; a sugar or sugar alcohol. In some embodiments, the sugar or sugar alcohol comprises sucrose, lactose, trehalose, dextran, erythritol, arabitol, xylitol, sorbitol, mannitol, or any combination thereof. In some embodiments, the one or more additives comprise a polymer. In some embodiments, the polymer comprises polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof.

The dsDNA suspected of comprising the target nucleic acid sequence can comprise a first strand and a second strand complementary to each other. In some embodiments, amplifying the target nucleic acid sequence comprises: amplifying the target nucleic acid sequence in an isothermal amplification condition, wherein the amplifying comprises contacting the ssDNA with: i) a first primer and a second primer, wherein the first primer is capable of hybridizing to a sequence of the first strand of the target nucleic acid sequence, and the second primer is capable of hybridizing to a sequence of the second strand of the target nucleic acid sequence; and ii) an enzyme having a hyperthermophile polymerase activity, thereby generating a nucleic acid amplification product, wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

The enzyme having a hyperthermophile polymerase activity can have an amino acid sequence that is at least about 90% or 95% identical to the sequence of SEQ ID NO: 1 or a functional fragment thereof, for example the enzyme having a hyperthermophile polymerase activity can be a polymerase comprising the amino acid sequence of SEQ ID NO: 1. In some embodiments, the enzyme having a hyperthermophile polymerase activity has low or no exonuclease activity.

The first primer and/or the second primer can be about 8 to 16 bases long. The first primer and/or the second primer can comprise one or more of DNA bases, modified DNA bases, or a combination thereof. The nucleic acid amplification product can be about 20 to 40 bases long (e.g., about 20, 21, 22, 23, 24, 25, 26, 27, 28, 29, 30, 31, 32, 33, 34, 35, 36, 37, 38, 39, or 40 bases long). The spacer sequence can comprise a portion of the target nucleic acid sequence. The spacer sequence can be 1 to 10 bases long. The dsDNA can comprise genomic DNA (gDNA), plasmid DNA, or both.

The biological entities can comprise one or more of prokaryotic cells, eukaryotic cells, viral particles, exosomes, protoplasts, and microvesicles. In some embodiments, the biological entities comprise a virus, a bacteria, a fungi, a protozoa, portions thereof, or any combination thereof. The target nucleic acid sequence can be a nucleic acid sequence of a virus, bacteria, fungi, or protozoa. In some embodiments, the sample nucleic acids are derived from a virus, bacteria, fungi, or protozoa. The virus can be Hepatitis B virus, Herpes Simplex, Herpesvirus 6, Herpesvirus 7, Epstein-Barr Virus, Cytomegalo-virus, Varicella-Zoster Virus, JC Virus, Parvovirus B19, Rotavirus, Human Adenovirus, or Genital Human Papillomavirus (HPV). The bacteria can be Salmonella enterica, Streptococcus pyogenes, Clostridium difficile, Streptococcus agalactiae, Mycobacteria tuberculosis, Rickettsia rickettsii, Ehrlichia chaffeensis, Borrelia burgdorferi, Yersinia pestis, Treponema pallidum, Chlamydia trachomatis, Chlamydia pneumoniae, Mycoplasma pneumoniae, Mycoplasma sp., Legionella pneumophila, Legionella dumoffii, Mycoplasma fermentans, Ehrlichia sp., Haemophilus influenzae, Neisseria meningitidis, Neisseria gonorrhoeae, Streptococcus pneumonia, S. agalactiae, or Listeria monocytogenes. The fungi can be Coccidioides posadasii, Cryptococcus neoformans, Pneumocystis carinii, Histoplasma capsulatum, Blastomyces dermatitidis, Coccidioides immitis, or Trichophyton rubrum. The protozoa can be Trichomonas vaginalis, Trypanosoma cruzi, Leishmania sp., Plasmodium, Entamoeba histolytica, Babesia microti, Giardia lamblia, Cyclospora sp., or Eimeria sp.

The sample can be a biological sample or an environmental sample. The environmental sample can be, or can be obtained from, a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof. The biological sample can be, or can be obtained from, a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof.

Contacting the sample with the acidic composition can be performed at a temperature of about 18° C., 19° C., 20° C., 21° C., 22° C., 23° C., 24° C., 25° C., 26° C., 27° C., 28° C., 29° C., 30° C., 31° C., 32° C., 33° C., 34° C., 35° C., 36° C., 37° C., 38° C., 39° C., 40° C., 41° C., 42° C., 43° C., 44° C., 45° C., 46° C., 47° C., 48° C., 49° C., 50° C., 51° C., 52° C., 53° C., 54° C., 55° C., 56° C., 57° C., 58° C., 59° C., 60° C., 61° C., 62° C., 63° C., 64° C., 65° C., 66° C., 67° C., 68° C., 69° C., 70° C., 71° C., 72° C., 73° C., 74° C., 75° C., 76° C., 77° C., 78° C., 79° C., 80° C., 81° C., 81.5° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or a number or a range between any two of these values. Contacting the sample with the acidic composition can be performed at a temperature of about 78° C. Contacting the sample with the acidic composition can be performed at a temperature in the range of about 18° C. to about 25° C. (e.g., room temperature). Contacting the sample with the acidic composition can be performed for a period of about 5 seconds, 10 seconds, 20 seconds, 30 seconds, 40 seconds, 50 seconds, 1 minute, 2 minutes, 3 minutes, 4 minutes, 5 minutes, 6 minutes, 7 minutes, 8 minutes, 9 minutes, 10 minutes, 20 minutes, 30 minutes, 40 minutes, 50 minutes, or about 60 minutes, or a number or a range between any two of these values.

The buffering agent can comprise MES, MOPS, MOPSO, Tris, HEPES, phosphate, acetate, citrate, succinate, ammonium buffers, or any combination thereof. In some embodiments, the neutral mixture has a pH in the range of about 7 to about 9 (e.g., about 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, about 9.0, or a number or a range between any two of these values). In some embodiments, the neutral mixture has a pH of about 8.8. The buffering agent can be present in the neutral mixture at a concentration of about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, or a number or a range between any two of these values. The buffering agent(s) present in the reagent composition (e.g., a dried composition, a wet composition) and neutral mixture provided herein can vary, and can include, for example, citrate buffer, maleate, phosphate, glycine, glycylglycine, carbonate, ethanolamine, ADA, imidazole, hydrazine, HEPBS, TABS, borate, N-(2-Acetamido)-aminoethanesulfonic acid (ACES), salt of acetic acid (acetate), N-(2-Acetamido)-iminodiacetic acid (ADA), 2-Aminoethanesulfonic acid, Taurine (AES), Ammonia, 2-Amino-2-methyl-1-propanol (AMP), 2-Amino-2-methyl-1,3-propanediol, (Ammediol or AMPD), N-(1,1-Dimethyl-2-hydroxyethyl)-3-amino-2-hydroxypropanesulfonic acid (AMPSO), N,N-Bis-(2-hydroxyethyl)-2-aminoethanesulfonic acid (BES), sodium bicarbonate, N,N′-Bis(2-hydroxyethyl)-glycine (Bicine), [Bis-(2-hydroxyethyl)-imino]-tris-(hydroxymethylmethane) (BIS-Tris), 1,3 Bis[tris(hydroxymethyl)-methylamino]propane) (BIS-Tris-Propane), boric acid, Dimethylarsinic acid (Cacodylate), 3-(Cyclohexylamino)-propanesulfonic acid (CAPS), 3-(cyclohexylamino)-2-hydroxy-1-propanesulfonic acid (CAPSO), sodium carbonate, cyclohexylaminoethanesulfonic acid (CHES), salt of acid citric (Citrate), 3-[N-Bis(hydroxyethyl)amino]-2-hydroxypropanesulfonic acid (DIPSO), Formate Salt of formic acid, glycine, glycylglycine, N-(2-Hydroxyethyl)-piperazine-N′-ethanesulfonic acid (HEPES), N-(2-Hydroxyethyl)-piperazine-N′-3-propanesulfonic acid (HEPPS, EPPS), N-(2-Hydroxyethyl)-piperazine-N′-2-hydroxypropanesulfonic acid (HEPPSO), salt of malic acid (malate), maleate salt of maleic acid, 2-(N-Morpholino)-ethanesulfonic acid (MES), 3-(N-Morpholino)-propanesulfonic acid (MOPS), 3-(N-Morpholino)-2-hydroxypropanesulfonic acid (MOPSO), Salt of phosphoric acid (Phosphate), Piperazine-N,N′-bis(2-ethanesulfonic acid) (PIPES), Piperazine-N,N′-bis(2-hydroxypropanesulfonic acid) (POPSO), pyridine, salt of succinic acid (succinate), 3-{[Tris(hydroxymethyl)-methyl]-amino}-propanesulfonic acid (TAPS), 3-[N-Tris(hydroxymethyl)-methylamino]-2-hydroxypropanesulfonic acid (TAPSO), Triethanolamine (TEA), 2-[Tris(hydroxymethyl)-methylamino]-ethanesulfonic acid (TES), N-[Tris(hydroxymethyl)-methyl]-glycine (Tricine), and Tris(hydroxymethyl)-aminomethane (Tris), or any combination thereof.

The methods, compositions, and kits disclosed herein can pair the use of acidic separation with the amplification of short target nucleic acid sequences (e.g., about 30 nucleotides in length). The target nucleic acid sequence can have a length of no longer than about 20 nucleotides, no longer than about 30 nucleotides, no longer than about 40 nucleotides, no longer than about 50 nucleotides, no longer than about 60 nucleotides, or no longer than about 100 nucleotides (e.g., 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 nucleotides). The target nucleic acid sequence can have a length of about 30 nucleotides.

The amplifying can be performed in an isothermal amplification condition. The isothermal amplification condition can comprise a constant temperature of about 30° C. to about 72° C. The isothermal amplification condition can comprise a constant temperature of about 67° C. The amplifying can be performed for a period of about 5, 6, 7, 8, 9, 10, 20, 30, 40, 50, or about 60 minutes, or a number or a range between any two of these values. The amplifying can be performed for a period of about 15 minutes. The amplifying can be performed in helicase-free, single-stranded binding protein-free, cleavage agent-free, and recombinase-free, isothermal amplification conditions.

In some embodiments, step (d) further comprises determining the amount of the dsDNA that comprises the target nucleic acid sequence in the sample. Detecting the nucleic acid amplification product can comprise use of a real-time detection method. Detecting the nucleic acid amplification product can comprise contacting the nucleic acid amplification product with a signal-generating oligonucleotide capable of hybridizing to the nucleic acid amplification product, wherein the single-generating oligonucleotide comprises a fluorophore, a quencher, or both. Detecting the nucleic acid amplification product can comprise detecting a fluorescent signal. The fluorescent signal can be from a molecular beacon. The method can be performed in a single reaction vessel.

The amplifying can comprise multiplex amplification of two or more target nucleic acid sequences. The detecting can comprise multiplex detection of two or more nucleic acid amplification products derived from said two or more target nucleic acid sequences. In some embodiments, the two or more target nucleic acid sequences are specific to two or more different organisms. In some embodiments, the two or more different organisms comprise Chlamydia trachomatis and Neisseria gonorrhoeae.

In some embodiments of the methods described herein, the amplifying/amplification does not comprise using any enzyme other than the enzyme having a hyperthermophile polymerase activity. The amplifying can comprise one or more of the following: Archaeal Polymerase Amplification (APA), loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA). In some embodiments, the amplifying does not comprise one or more of the following: Archaeal Polymerase Amplification (APA), LAMP, HDA, RPA, SDA, NASBA, TMA, NEAR, RCA, MDA, RAM, cHDA, SPIA, SMART, 3SR, GEAR and IMDA.

In some embodiments, the method does not comprise one or more of the following: (i) dilution of the acidic mixture (e.g., treated sample); (ii) dilution of the neutral mixture (e.g., amplification reaction mixture); (iii) heat denaturation of the acidic mixture; (iv) sonication of the acidic mixture; (v) sonication of the neutral mixture; (vi) the addition of ribonuclease inhibitors to the acidic mixture; (vii) the addition of ribonuclease inhibitors to the neutral mixture; (viii) purification of the sample; (ix) purification of the sample nucleic acids; (x) purification of the nucleic acid amplification product; (xi) removal of the one or more surfactants from the acidic mixture or the neutral mixture; (xii) heating denaturing and/or enzymatic denaturing of the sample nucleic acids prior to and/or during amplification; (xiii) the addition of ribonuclease H to the acidic mixture or the neutral mixture; (xiv) contacting the sample, the acidic mixture, or the neutral mixture with a helicase, a single-stranded binding protein, a nicking enzyme, a restriction enzyme, or a recombinase; (xv) contacting the sample, the acidic mixture, or the neutral mixture with glycerol, formamide, or urea; (xvi) purifying the ssDNA from the acidic mixture or the neutral mixture; (xvii) a heat denaturing step; and (xviii) enriching, purifying and/or isolating the dsDNA.

Compositions, kits, and methods for nucleic acid detection wherein lytic agents employed to lyse biological entities (e.g., viral particles, bacteria) are prevented from inactivating amplification reagents (e.g., polymerases) and wherein the deleterious activity of ribonucleases is inhibited at some or all stages are described in PCT Patent Application Publication No. WO2022198086A1, the content of which is incorporated herein by reference in its entirety. In some embodiments, the acidic compositions (e.g., lysis buffers) provided herein comprise one or more reducing agents (e.g., DTT) as described therein and/or the reagent compositions provided herein comprise one or more protectants (e.g., a cyclodextrin compound) as described therein. In some embodiments, the disclosed methods and compositions enable isothermal amplification and real-time detection of nucleic acids without a need for sample separation or purification for point-of-care molecular diagnostics for direct pathogen lysis in clinical samples. There is provided, in some embodiments, an acidic composition (e.g., a lysis buffer) containing a potent ionic detergent that can be used to lyse pathogens in clinical samples. In some embodiments, an acidic composition (e.g., a lysis buffer) can comprise a chelating agent. The amplification reagents can comprise a protectant against the lysis reagent, and can be dried (e.g., lyophilized, heat dried) and used for the amplification of the released nucleic acids in point-of-care settings.

The methods and compositions provided herein can be applied to other amplification methods for sample preparation without purification or separation, for example, PCR, RT-PCR, or other isothermal amplification methods. The methods and compositions provided herein can also be applied to genome sequencing methods or any nucleic acids (DNA or RNA) amplification or detection methods that require a sample preparation step. The methods and compositions provided herein can also find use in genotyping, diagnostics and forensics. The disclosed methods and compositions are not limited to isothermal amplification methods, but rather can be applied to other amplification/detection methods, for example, RT-PCR, WGS sequencing, and can also be applied to RNA purification/extraction without separation.

Disclosed herein include methods for detecting a target nucleic acid sequence in a sample. In some embodiments, the method comprises: (a) contacting a sample comprising biological entities with a lysis buffer (e.g., an acidic composition) provided herein to generate an acidic mixture (e.g., treated sample), wherein the lysis buffer is capable of lysing biological entities to release sample nucleic acids comprised therein, wherein the sample nucleic acids are suspected of comprising a target nucleic acid sequence. The method can comprise: (b) contacting a reagent composition (e.g., a dried composition, a wet composition) with the acidic mixture to generate a neutral mixture (e.g., amplification reaction mixture), wherein the reagent composition comprises one or more amplification reagents. The method can comprise: (c) amplifying a target nucleic acid sequence in the neutral mixture, thereby generating a nucleic acid amplification product. The method can comprise: (d) detecting the nucleic acid amplification product, wherein the detecting is performed in less than or less than about 20 minutes (e.g., less than or less than about 20, 19, 18, 17, 16, 15, 14, 13, 12, 11, 10, 9, 8, 7, 6, 5, 4, 3, 2, 1 minute(s), or a number or a range between any two of these values) from the time the reagent composition is contacted with the acidic mixture. In some embodiments, steps (b) and (c) are performed concurrently (e.g., the amplification begins once the contacting of the reagent composition and the acidic mixture has occurred). In some embodiments, the reagent composition comprises two or more dried compositions (comprising the same or different components) or two or more wet compositions (comprising the same or different components). In some embodiments, the lysis buffer comprises two or more lysis buffers (comprising the same or different components).

The sample nucleic acids can comprise sample ribonucleic acids and/or sample deoxyribonucleic acids. The sample ribonucleic acids can comprise a cellular RNA, a mRNA, a microRNA, a bacterial RNA, a viral RNA, or any combination thereof. The one or more amplification reagents can comprise a reverse transcriptase and/or an enzyme having a hyperthermophile polymerase activity. In some embodiments, the enzyme having a hyperthermophile polymerase activity has a reverse transcriptase activity. Contacting the reagent composition (e.g., a dried composition, a wet composition) with the acidic mixture (e.g., treated sample) can comprise dissolving the reagent composition in the acidic mixture. The reagent composition can comprise one or more of a reverse transcriptase, an enzyme having a hyperthermophile polymerase activity, a first primer, a second primer, and a reverse transcription primer. The amplifying can be performed in an isothermal amplification condition. Detecting the nucleic acid amplification product can comprise use of a real-time detection method. The sample nucleic acids can comprise a nucleic acid comprising the target nucleic acid sequence. The target nucleic acid sequence can comprise a first strand and a second strand complementary to each other.

Amplifying the target nucleic acid sequence can comprise: amplifying a target nucleic acid sequence comprising a first strand and a second strand complementary to each other in an isothermal amplification condition, wherein the amplifying comprises contacting a nucleic acid comprising the target nucleic acid sequence with: i) a first primer and a second primer, wherein the first primer is capable of hybridizing to a sequence of the first strand of the target nucleic acid sequence, and the second primer is capable of hybridizing to a sequence of the second strand of the target nucleic acid sequence; and ii) an enzyme having a hyperthermophile polymerase activity, thereby generating a nucleic acid amplification product, wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

In some embodiments, the amplifying does not comprise using any enzyme other than the enzyme having a hyperthermophile polymerase activity, and the amplifying step does not comprise denaturing the nucleic acid. In some embodiments, the method does not comprise contacting the nucleic acid with a single-stranded DNA binding protein prior to or during step (c). In some embodiments, the method does not comprise thermal or enzymatic denaturation of the sample nucleic acid.

The nucleic acid can be a dsDNA. The nucleic acid can be a product of reverse transcription reaction. The nucleic acid can be a product of reverse transcription reaction generated from sample ribonucleic acids. Step (c) can comprise generating the nucleic acid by a reverse transcription reaction.

The sample nucleic acids can comprise sample ribonucleic acids. The method can comprise contacting sample ribonucleic acids with a reverse transcriptase and/or a reverse transcription primer to generate a cDNA. Amplifying the target nucleic acid sequence can comprise: (c1) contacting sample ribonucleic acids with a reverse transcriptase and/or a reverse transcription primer to generate a cDNA; (c2) contacting the cDNA with an enzyme having a hyperthermophile polymerase activity to generate a dsDNA, wherein the dsDNA comprises a target nucleic acid sequence, and wherein the target nucleic acid sequence comprises a first strand and a second strand complementary to each other; (c3) amplifying the target nucleic acid sequence under an isothermal amplification condition, wherein the amplifying comprises contacting the dsDNA with: (i) a first primer and a second primer, wherein the first primer is capable of hybridizing to a sequence of the first strand of the target nucleic acid sequence, and the second primer is capable of hybridizing to a sequence of the second strand of the target nucleic acid sequence; and (ii) the enzyme having a hyperthermophile polymerase activity, thereby generating a nucleic acid amplification product, wherein the nucleic acid amplification product comprises: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

In some embodiments, the method does not comprise using any enzymes other than the reverse transcriptase and the enzyme having a hyperthermophile polymerase activity. Step (d) further can comprise determining the amount of the dsDNA and/or nucleic acid that comprises the target nucleic acid sequence in the sample. The enzyme having a hyperthermophile polymerase activity can have an amino acid sequence that is at least about 90%, or at least about 95%, identical to the amino acid sequence of SEQ ID NO: 1 or a functional fragment thereof. The enzyme having a hyperthermophile polymerase activity can be a polymerase comprising, or consisting of, the amino acid sequence of SEQ ID NO: 1. In some embodiments, the enzyme having a hyperthermophile polymerase activity has low or no exonuclease activity.

Amplifying the target nucleic acid sequence can be performed at a constant temperature of about 55° C. to about 75° C., for example about 65° C. The first primer, the second primer, or both can be about 8 to 16 bases long. The first primer, the second primer, or both can comprise one or more of DNA bases, modified DNA bases, or a combination thereof. The nucleic acid amplification product can be about 20 to 40 bases long. The spacer sequence can comprise a portion of the target nucleic acid sequence. The spacer sequence can be 1 to 10 bases long.

In some embodiments, the method comprises: contacting the nucleic acid amplification product with a signal-generating oligonucleotide capable of hybridizing to the amplification product. The signal-generating oligonucleotide can comprise a fluorophore, a quencher, or both. Detecting the nucleic acid amplification product can comprise detecting a fluorescent signal. The fluorescent signal can be from a molecular beacon. The method can be performed in a single reaction vessel. The sample ribonucleic acids can be contacted with the reverse transcriptase and the enzyme having a hyperthermophile polymerase activity simultaneously. The sample ribonucleic acids can be contacted with the reverse transcriptase, the enzyme having a hyperthermophile polymerase activity, and the first and second primers simultaneously. In some embodiments, the sample ribonucleic acids are contacted with the reverse transcriptase, the enzyme having a hyperthermophile polymerase activity, the first primer, the second primer, and the reverse transcription primer simultaneously. Reverse transcription of the sample ribonucleic acids can occur by the addition of a reverse transcription primer. In some embodiments, the reverse transcription primer is an oligo (dT) primer, random hexanucleotide primer, or a target-specific oligonucleotide primer. In some embodiments, oligo (dT) primers are 12-18 nucleotides in length and bind to the endogenous poly(A)+ tail at the 3′ end of mRNA. Random hexanucleotide primers can bind to sample ribonucleic acids at a variety of complementary sites. Target-specific oligonucleotide primers typically selectively prime the sample ribonucleic acids of interest. In some embodiments, the first primer and/or second primer is a reverse transcription primer.

The amplifying step can comprise multiplex amplification of two or more target nucleic acid sequences. The detecting step can comprise multiplex detection of two or more nucleic acid amplification products derived from said two or more target nucleic acid sequences. The two or more target nucleic acid sequences can be specific to two or more different organisms. The lysis buffers (e.g., acidic compositions) provided herein can be employed upstream of a variety of amplification reactions, such as, for example, isothermal amplification reactions.

In some embodiments, the method does not comprise one or more, or any, of the following: (i) dilution of the acidic mixture (e.g., treated sample); (ii) dilution of the neutral mixture (e.g., amplification reaction mixture); (iii) heat denaturation of the acidic mixture; (iv) sonication of the acidic mixture; (v) sonication of the neutral mixture; (vi) the addition of ribonuclease inhibitors to the acidic mixture; (vii) the addition of ribonuclease inhibitors to the neutral mixture; (viii) purification of the sample; (ix) purification of the sample nucleic acids; (x) purification of the nucleic acid amplification product; (xi) removal of the one or more lytic agents (e.g., one or more surfactants) from the acidic mixture or the neutral mixture; (xii) heat and/or enzymatic denaturing the sample nucleic acids prior to or during amplification; and (xiii) the addition of ribonuclease H to the acidic mixture or neutral mixture. In some embodiments, the sample is held at an amplification temperature (e.g., 67° C.). In some embodiments, the sample (e.g. a sample comprising RNA) is held at temperature between room temperature and reaction temperature for 1-2 minutes before the amplification reaction to facilitate reverse transcription reaction.

In some embodiments, step (a), step (b), step (c), and/or step (d) is performed for a period of about 20 minutes, 15 minutes, 10 minutes, 5 minutes, 2.5 minutes, or about 1 minute. In some embodiments, step (a), step (b), step (c), and/or step (d) comprises sonication, osmotic shock, chemical treatment, heating, or any combination thereof.

The term “isothermal amplification reaction” shall be given its ordinary meaning and shall also include reactions wherein the temperature does not significantly change during the reaction. In some embodiments, the temperature of the isothermal amplification reaction does not deviate by more than 10° C., for example, by not more than 5° C. and by not more than 2° C. during the main enzymatic reaction step where amplification takes place. Depending on the method of isothermal amplification of nucleic acids, different enzymes can be used for amplification. Exemplary isothermal amplification compositions and methods are described in WO2017176404, the content of which is incorporated herein by reference in its entirety.

Disclosed herein include methods for amplifying nucleic acids. In some embodiments, the method comprises: contacting sample nucleic acid under isothermal amplification conditions with components comprising a) at least one oligonucleotide, which at least one oligonucleotide comprises a polynucleotide complementary to a target sequence in the sample nucleic acid, and b) at least one component providing hyperthermophile polymerase activity, thereby generating a nucleic acid amplification product. In some embodiments, the method comprises: contacting sample nucleic acid under isothermal amplification conditions with a) non-enzymatic components comprising at least one oligonucleotide, which at least one oligonucleotide comprises a polynucleotide complementary to a target sequence in the sample nucleic acid, and b) an enzymatic component consisting of a hyperthermophile polymerase or a polymerase comprising an amino acid sequence that is at least about 90% identical or at least 95% identical to a hyperthermophile polymerase, thereby generating a nucleic acid amplification product. In some embodiments, the method comprises: contacting sample nucleic acid under isothermal amplification conditions with a) non-enzymatic components comprising at least one oligonucleotide, which at least one oligonucleotide comprises a polynucleotide complementary to a target sequence in the sample nucleic acid, and b) enzymatic activity consisting of i) hyperthermophile polymerase activity and, optionally, ii) reverse transcriptase activity, thereby generating a nucleic acid amplification product.

Disclosed herein include methods for processing nucleic acids. In some embodiments, the method comprises: amplifying nucleic acid, wherein the amplifying comprises contacting sample nucleic acid under isothermal amplification conditions with a) at least one oligonucleotide, which at least one oligonucleotide comprises a polynucleotide complementary to a target sequence in the sample nucleic acid, and b) at least one component providing hyperthermophile polymerase activity, thereby generating a nucleic acid amplification product. In some embodiments, the method for processing nucleic acids comprises: amplifying nucleic acid, wherein the amplifying comprises contacting sample nucleic acid under isothermal amplification conditions with a) non-enzymatic components comprising at least one oligonucleotide, which at least one oligonucleotide comprises a polynucleotide complementary to a target sequence in the sample nucleic acid, and b) an enzymatic component consisting of a hyperthermophile polymerase or a polymerase comprising an amino acid sequence that is at least about 90% identical or at least 95% identical to a hyperthermophile polymerase, thereby generating a nucleic acid amplification product. In some embodiments, the method for processing nucleic acids comprises: amplifying nucleic acid, wherein the amplifying comprises contacting sample nucleic acid under isothermal amplification conditions with a) non-enzymatic components comprising at least one oligonucleotide, which at least one oligonucleotide comprises a polynucleotide complementary to a target sequence in the sample nucleic acid, and b) enzymatic activity consisting of i) hyperthermophile polymerase activity and, optionally, ii) reverse transcriptase activity, thereby generating a nucleic acid amplification product. In some embodiments, the enzymatic activity consists of i) hyperthermophile polymerase activity, and ii) reverse transcriptase activity.

Disclosed herein include methods for determining the presence, absence or amount of a target sequence in sample nucleic acid. In some embodiments, the method comprises: a) amplifying a target sequence in the sample nucleic acid, wherein: the target sequence comprises a first strand and a second strand, the first strand and second strand are complementary to each other, and the amplifying comprises contacting sample nucleic acid under helicase-free isothermal amplification conditions with: i) a first oligonucleotide and a second oligonucleotide, wherein the first oligonucleotide comprises or consists of a first polynucleotide continuously complementary to a sequence in the first strand, and the second oligonucleotide comprises or consists of a second polynucleotide continuously complementary to a sequence in the second strand; and ii) at least one component providing a hyperthermophile polymerase activity, thereby generating a nucleic acid amplification product, wherein the nucleic acid amplification product comprises or consists of 1) a first nucleotide sequence that is continuously complementary to or substantially identical to the first polynucleotide of the first oligonucleotide, 2) a second nucleotide sequence that is continuously complementary to or substantially identical to the second polynucleotide of the second oligonucleotide, and 3) a spacer sequence comprising 1 to 10 bases, and the spacer sequence is flanked by the first nucleotide sequence and the second nucleotide sequence; and b) detecting the nucleic acid amplification product, wherein detecting the nucleic acid amplification product comprises use of a real-time detection method and is performed in 10 minutes or less from the time the sample nucleic acid is contacted with (a) (i) and (a) (ii), whereby the presence, absence or amount of a target sequence in sample nucleic acid is determined.

Disclosed herein include kits for determining the presence, absence or amount of a target sequence in sample nucleic acid. In some embodiments, the kit comprises: a) components for amplifying a target sequence in the sample nucleic acid under helicase-free isothermal amplification conditions, which components comprise: i) a first oligonucleotide and a second oligonucleotide, wherein the first oligonucleotide comprises or consists of a first polynucleotide continuously complementary to a sequence in a first strand of the target sequence, and the second oligonucleotide comprises or consists of a second polynucleotide continuously complementary to a sequence in a second strand of the target sequence, which first strand and second strand of the target sequence are complementary to each other; and ii) at least one component providing a hyperthermophile polymerase activity; and b) at least one component providing real-time detection activity for a nucleic acid amplification product.

The enzymatic activity can comprise, or consist of, i) hyperthermophile polymerase activity, and ii) reverse transcriptase activity. In some embodiments, the method does not comprise enzymatic denaturation and/or heat denaturation of the sample nucleic acid prior to or during amplification. In some embodiments, the sample nucleic acid is not contacted with an endonuclease prior to or during amplification. In some embodiments, the sample nucleic acid is not contacted with an unwinding agent prior to or during amplification. In some embodiments, the sample nucleic acid is not contacted with a helicase prior to or during amplification. In some embodiments, the sample nucleic acid is not contacted with a recombinase prior to or during amplification. In some embodiments, the sample nucleic acid is not contacted with a single-stranded DNA binding protein prior to or during amplification. In some embodiments, the sample nucleic acid is unmodified prior to amplification. In some embodiments, the unmodified sample nucleic acid is from disrupted cells. In some embodiments, the sample nucleic acid comprises DNA. In some embodiments, the sample nucleic acid comprises genomic DNA. In some embodiments, the sample nucleic acid comprises RNA. In some embodiments, the sample nucleic acid comprises viral RNA. In some embodiments, the sample nucleic acid comprises bacterial RNA. The sample nucleic acid can comprise single-stranded nucleic acid, double-stranded nucleic acid, or both. For example, the double-stranded nucleic acid can comprise a first strand and a second strand. In some embodiments, the at least one oligonucleotide comprises or consists of a first oligonucleotide and a second oligonucleotide. In some embodiments, the first oligonucleotide and the second oligonucleotide each comprise 8 to 16 bases. In some embodiments, the first oligonucleotide comprises or consists of a first polynucleotide complementary to a target sequence in the first strand of the sample nucleic acid, and the second oligonucleotide comprises a second polynucleotide complementary to a target sequence in the second strand of the sample nucleic acid. In some embodiments, sample nucleic acid is obtained from a subject prior to amplification. In some embodiments, unpurified sample nucleic acid is amplified. In some embodiments, purified sample nucleic acid is amplified. In some embodiments, the method further comprises purifying sample nucleic acid prior to amplification.

In some embodiments, the hyperthermophile polymerase activity is provided by a hyperthermophile polymerase or functional fragment thereof. In some embodiments, the hyperthermophile polymerase activity is provided by a polymerase comprising an amino acid sequence that is at least about 90% identical to a hyperthermophile polymerase or functional fragment thereof. In some embodiments, the hyperthermophile polymerase activity is provided by an Archaea hyperthermophile polymerase or functional fragment thereof. In some embodiments, the hyperthermophile polymerase activity is provided by a polymerase comprising an amino acid sequence of SEQ ID NO: 1 or functional fragment thereof, or a polymerase comprising an amino acid sequence that is at least about 90% identical to the amino acid sequence of SEQ ID NO: 1 or functional fragment thereof. In some embodiments, the hyperthermophile polymerase activity is provided by a polymerase having low or no exonuclease activity. In some embodiments, the amplification is performed at a constant temperature of about 55° C. to about 75° C., for example a constant temperature of about 55° C. to about 65° C. or about 65° C. or about 60° C.

In some embodiments, the nucleic acid amplification product is detectable in 20, 15, 14, 13, 12, 11, or 10 minutes or less. In some embodiments, the nucleic acid amplification product comprises, or consists of, a polynucleotide that is continuously complementary to or substantially identical to a target sequence in the sample nucleic acid. The nucleic acid amplification product can be about 20 to 40 bases long. In some embodiments, the nucleic acid amplification product comprises or consists of i) a first nucleotide sequence that is continuously complementary to or substantially identical to the first polynucleotide of the first oligonucleotide, ii) a second nucleotide sequence that is continuously complementary to or substantially identical to the second polynucleotide of the second oligonucleotide, and iii) a spacer sequence, wherein the spacer sequence is flanked by the first nucleotide sequence and the second nucleotide sequence. In some embodiments, the spacer sequence comprises 1 to 10 bases, for example 1 to 5 bases. In some embodiments, the spacer sequence is not complementary to or identical to the first polynucleotide of the first oligonucleotide and is not complementary to or identical to the second polynucleotide of the second oligonucleotide. In some embodiments, the spacer sequence is continuously complementary to or substantially identical to a portion of a target sequence in the sample nucleic acid.

In some embodiments, the method further comprises detecting the nucleic acid amplification product. In some embodiments, detecting the nucleic acid amplification product is performed in 10 minutes or less from the time the sample nucleic acid is contacted with the component providing the hyperthermophile polymerase activity and the at least one oligonucleotide. In some embodiments, detecting the nucleic acid amplification product comprises use of a real-time detection method. In some embodiments, detecting the nucleic acid amplification product comprises detection of a fluorescent signal. In some embodiments, the fluorescent signal is from a molecular beacon. In some embodiments, the method further comprises contacting the nucleic acid amplification product with a signal generating oligonucleotide that comprises i) a polynucleotide complementary to a sequence in the amplification product, and ii) a fluorophore and a quencher. In some embodiments, one or more of the at least one oligonucleotide comprise a polynucleotide not complementary to a sequence in the sample nucleic acid that hybridizes to a signal generating oligonucleotide, and wherein the method further comprises contacting the amplification product with the signal generating oligonucleotide that comprises a fluorophore and a quencher. In some embodiments, the method is performed in a single reaction volume. In some embodiments, the method is performed in a single reaction vessel. In some embodiments, the method comprises multiplex amplification.

In some embodiments, the enzymatic activity consists of i) hyperthermophile polymerase activity, and ii) reverse transcriptase activity. In some embodiments, the first oligonucleotide comprises, or consists of, a first polynucleotide complementary to a target sequence in the first strand of the sample nucleic acid, and the second oligonucleotide comprises a second polynucleotide complementary to a target sequence in the second strand of the sample nucleic acid. In some embodiments, the first oligonucleotide comprises, or consists of, a first polynucleotide continuously complementary to a target sequence in the first strand of the sample nucleic acid, and the second oligonucleotide comprises a second polynucleotide continuously complementary to a target sequence in the second strand of the sample nucleic acid.

In some embodiments, the amplifying comprises contacting sample nucleic acid under helicase-free and recombinase-free isothermal amplification conditions. In some embodiments, the at least one component providing a hyperthermophile polymerase activity comprises, or consists of, a hyperthermophile polymerase or functional fragment thereof, or a polymerase comprising, or consisting of, an amino acid sequence that is at least about 90% identical to a hyperthermophile polymerase or functional fragment thereof. In some embodiments, part (a) (ii) further comprises at least one component providing a reverse transcriptase activity. In some embodiments, the at least one component providing hyperthermophile polymerase activity further provides a reverse transcriptase activity. In some embodiments, the sample nucleic acid is amplified under helicase-free and recombinase-free isothermal amplification conditions. In some embodiments, the real-time detection activity is provided by a molecular beacon. In some embodiments, kit further comprises instructions for carrying out a method provided herein for determining the presence, absence or amount of a target sequence in sample nucleic acid.

In some embodiments, the methods and components described herein comprise a storage-stable lysis buffer. In some embodiments, the lysis buffer is resistant to the formation of a precipitate for a period of time under a storage condition (e.g., storage-stable lysis buffer). Compositions, kits, and methods wherein lysis buffers resist precipitation are described in the U.S. Patent Application No. 63/307,092, entitled “NON-OPAQUE LYTIC BUFFER COMPOSITION FORMULATIONS”, filed on Feb. 5, 2022, the content of which is incorporated herein by reference in its entirety.

### Nucleic Acid, Subjects, Samples and Nucleic Acid Processing

Provided herein are methods and compositions for amplifying nucleic acid. The terms “nucleic acid” and “nucleic acid molecule” may be used interchangeably herein. The terms refer to nucleic acids of any composition, such as DNA (e.g., complementary DNA (cDNA), genomic DNA (gDNA) and the like), RNA (e.g., message RNA (mRNA), short inhibitory RNA (siRNA), ribosomal RNA (rRNA), tRNA, microRNA, and/or DNA or RNA analogs (e.g., containing base analogs, sugar analogs and/or a non-native backbone and the like), RNA/DNA hybrids and polyamide nucleic acids (PNAs), all of which can be in single- or double-stranded form, and unless otherwise limited, can encompass known analogs of natural nucleotides that can function in a similar manner as naturally occurring nucleotides. A nucleic acid can be, or can be from, a plasmid, phage, autonomously replicating sequence (ARS), centromere, artificial chromosome, chromosome, or other nucleic acid able to replicate or be replicated in vitro or in a host cell, a cell, a cell nucleus, a mitochondria, or cytoplasm of a cell. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, single nucleotide polymorphisms (SNPs), and complementary sequences as well as the sequence explicitly indicated. Specifically, degenerate codon substitutions may be achieved by generating sequences in which the third position of one or more selected (or all) codons is substituted with mixed-base and/or deoxyinosine residues. The term nucleic acid may be used interchangeably with locus, gene, cDNA, and mRNA encoded by a gene. The term also may include, as equivalents, derivatives, variants and analogs of RNA or DNA synthesized from nucleotide analogs, single-stranded (“sense” or “antisense”, “plus” strand or “minus” strand, “forward” reading frame or “reverse” reading frame, “forward” strand or “reverse” strand) and double-stranded polynucleotides. The term “gene” means the segment of DNA involved in producing a polypeptide chain; and generally includes regions preceding and following the coding region (leader and trailer) involved in the transcription/translation of the gene product and the regulation of the transcription/translation, as well as intervening sequences (introns) between individual coding segments (exons). A nucleotide or base generally refers to the purine and pyrimidine molecular units of nucleic acid (e.g., adenine (A), thymine (T), guanine (G), and cytosine (C)). For RNA, the base thymine is replaced with uracil. Nucleic acid length or size may be expressed as a number of bases.

In some embodiments of the methods provided herein, one or more nucleic acid targets are amplified. Target nucleic acids may be referred to as target sequences, target polynucleotides, and/or target polynucleotide sequences, and may include double-stranded and single-stranded nucleic acid molecules. Target nucleic acid may be, for example, DNA or RNA. Where the target nucleic acid is an RNA molecule, the molecule may be, for example, double-stranded, single-stranded, or the RNA molecule may comprise a target sequence that is single-stranded. Where the target nucleic acid is double stranded, the target nucleic acid generally includes a first strand and a second strand. A first strand and a second strand may be referred to as a forward strand and a reverse strand and generally are complementary to each other. Where the target nucleic acid is single stranded, a complementary strand may be generated, for example by polymerization and/or reverse transcription, rendering the target nucleic acid double stranded and having a first/forward strand and a second/reverse strand.

A target sequence may refer to either the sense or antisense strand of a nucleic acid sequence, and also may refer to sequences as they exist on target nucleic acids, amplified copies, or amplification products, of the original target sequence. A target sequence can be a subsequence within a larger polynucleotide. For example, a target sequence can be a short sequence (e.g., 20 to 50 bases) within a nucleic acid fragment, a chromosome, a plasmid, that is targeted for amplification. In some embodiments, a target sequence may refer to a sequence in a target nucleic acid that is complementary to an oligonucleotide (e.g., primer) used for amplifying a nucleic acid. Thus, a target sequence may refer to the entire sequence targeted for amplification or may refer to a subsequence in the target nucleic acid where an oligonucleotide binds. An amplification product may be a larger molecule that comprises the target sequence, as well as at least one other sequence, or other nucleotides. The amplification product can be about the same length as the target sequence, for example exactly the same length as the target sequence. The amplification product can comprise, or consist of, the target sequence.

The length of the target sequence, and/or the guanosine cytosine (GC) concentration (percent), may depend, in part, on the temperature at which an amplification reaction is run, and this temperature may depend, in part, on the stability of the polymerase(s) used in the reaction. Sample assays may be performed to determine an appropriate target sequence length and GC concentration for a set of reaction conditions. For example, where a polymerase is stable up to 60° C. to 65° C., then the target sequence may be, for example, from 19 to 50 nucleotides in length, or for example, from about 40 to 50, 20 to 45, 20 to 40, or 20 to 30 nucleotides in length. GC concentration under these conditions may be, for example, less than 60%, less than 55%, less than 50%, or less than 45%.

Target nucleic acid may include, for example, genomic nucleic acid, plasmid nucleic acid, mitochondrial nucleic acid, cellular nucleic acid, extracellular nucleic acid, bacterial nucleic acid and viral nucleic acid. In some embodiments, target nucleic acid may include genomic DNA, chromosomal DNA, plasmid DNA, mitochondrial DNA, a gene, any type of cellular RNA, messenger RNA, bacterial RNA, viral RNA or a synthetic oligonucleotide. Genomic nucleic acid can include any nucleic acid from any genome, for example, animal, plant, insect, viral and bacterial genomes (e.g., genomes present in spores). In some embodiments, genomic target nucleic acid is within a particular genomic locus or a plurality of genomic loci. A genomic locus can include any or a combination of open reading frame DNA, non-transcribed DNA, intronic sequences, extronic sequences, promoter sequences, enhancer sequences, flanking sequences, or any sequences considered associated with a given genomic locus.

The target sequence can comprise one or more repetitive elements (e.g., multiple repeat sequences, inverted repeat sequences, palindromic sequences, tandem repeats, microsatellites, minisatellites, and the like). In some embodiments, a target sequence is present within a sample nucleic acid (e.g., within a nucleic acid fragment, a chromosome, a genome, a plasmid) as a repetitive element (e.g., a multiple repeat sequence, an inverted repeat sequence, a palindromic sequence, a tandem repeat, a microsatellite repeat, a minisatellite repeat and the like). For example, a target sequence may occur multiple times as a repetitive element and one, some, or all occurrences of the target sequence within a repetitive element may be amplified (e.g., using a single pair of primers) using methods described herein. In some embodiments, a target sequence is present within a sample nucleic acid (e.g., within a nucleic acid fragment, a chromosome, a genome, a plasmid) as a duplication and/or a paralog.

Target nucleic acid can include microRNAs. MicroRNAs, miRNAs, or small temporal RNAs (stRNAs) are short (e.g., about 21 to 23 nucleotides long) and single-stranded RNA sequences involved in gene regulation. MicroRNAs may interfere with translation of messenger RNAs and are partially complementary to messenger RNAs. Target nucleic acid can include microRNA precursors such as primary transcript (pri-miRNA) and pre-miRNA stem-loop-structured RNA that is further processed into miRNA. Target nucleic acid can include short interfering RNAs (siRNAs), which are short (e.g., about 20 to 25 nucleotides long) and at least partially double-stranded RNA molecules involved in RNA interference (e.g., down-regulation of viral replication or gene expression).

Nucleic acid utilized in methods described herein can be obtained from any suitable biological specimen or sample, e.g., isolated from a sample obtained from a subject. A subject can be any living or non-living organism, including but not limited to a human, a non-human animal, a plant, a bacterium, a fungus, a virus, or a protist. Any human or non-human animal can be selected, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. A subject may be a male or female, and a subject may be any age (e.g., an embryo, a fetus, infant, child, adult).

A sample or test sample can be any specimen that is isolated or obtained from a subject or part thereof. Non-limiting examples of specimens include fluid or tissue from a subject, including, without limitation, blood or a blood product (e.g., serum, plasma, or the like), umbilical cord blood, bone marrow, chorionic villi, amniotic fluid, cerebrospinal fluid, spinal fluid, lavage fluid (e.g., bronchoalveolar, gastric, peritoneal, ductal, ear, arthroscopic), serum, plasma, urine, aspirate, biopsy sample, celocentesis sample, cells (e.g., blood cells) or parts thereof (e.g., mitochondrial, nucleus, extracts, or the like), washings of female reproductive tract, urine, feces, sputum, saliva, nasal mucous, prostate fluid, lavage, semen, lymphatic fluid, bile, tears, sweat, breast milk, breast fluid, hard tissues (e.g., liver, spleen, kidney, lung, or ovary), the like or combinations thereof. The term blood encompasses whole blood, blood product or any fraction of blood, such as serum, plasma, buffy coat, or the like as conventionally defined. Blood plasma refers to the fraction of whole blood resulting from centrifugation of blood treated with anticoagulants. Blood serum refers to the watery portion of fluid remaining after a blood sample has coagulated. Fluid or tissue samples often are collected in accordance with standard protocols hospitals or clinics generally follow. For blood, an appropriate amount of peripheral blood (e.g., between 3-40 milliliters) often is collected and can be stored according to standard procedures prior to or after preparation.

A sample or test sample can include samples containing spores, viruses, cells, nucleic acid from prokaryotes or eukaryotes, or any free nucleic acid. For example, a method described herein may be used for detecting nucleic acid on the outside of spores (e.g., without the need for lysis). A sample can be isolated from any material suspected of containing a target sequence, such as from a subject described above. In some embodiments, a target sequence is present in air, plant, soil, or other materials suspected of containing biological organisms.

Nucleic acid can be derived (e.g., isolated, extracted, purified) from one or more sources by methods known in the art. Any suitable method can be used for isolating, extracting and/or purifying nucleic acid from a biological sample, including methods of DNA preparation in the art, and various commercially available reagents or kits, such as Qiagen's QIAamp Circulating Nucleic Acid Kit, QiaAmp DNA Mini Kit or QiaAmp DNA Blood Mini Kit (Qiagen, Hilden, Germany), GenomicPrep™ Blood DNA Isolation Kit (Promega, Madison, Wis.), GFX™ Genomic Blood DNA Purification Kit (Amersham, Piscataway, N.J.), and the like or combinations thereof. U.S. Pat. No. 7,888,006 provides DNA purification methods and does not disclose the compositions (e.g., lysis buffers, protectants) and methods provided herein

In some embodiments, a cell lysis procedure is performed. Cell lysis can be performed prior to initiation of an amplification reaction described herein (e.g., to release DNA and/or RNA from cells for amplification). Cell lysis procedures and reagents are known in the art and may be performed by chemical (e.g., detergent, hypotonic solutions, enzymatic procedures, and the like, or combination thereof), physical (e.g., French press, sonication, and the like), or electrolytic lysis methods. For example, chemical methods generally employ lysing agents to disrupt cells and extract nucleic acids from the cells, followed by treatment with chaotropic salts. In some embodiments, cell lysis comprises use of detergents (e.g., ionic, nonionic, anionic, zwitterionic). In some embodiments, cell lysis comprises use of ionic detergents (e.g., sodium dodecyl sulfate (SDS), sodium lauryl sulfate (SLS), deoxycholate, cholate, sarkosyl). Physical methods such as freeze/thaw followed by grinding, the use of cell presses and the like also may be useful. High salt lysis procedures also may be used. For example, an alkaline lysis procedure may be utilized. The latter procedure traditionally incorporates the use of phenol-chloroform solutions, and an alternative phenol-chloroform-free procedure involving three solutions may be utilized. In the latter procedures, one solution can contain 15 mM Tris, pH 8.0; 10 mM EDTA and 100 μg/ml Rnase A; a second solution can contain 0.2N NaOH and 1% SDS; and a third solution can contain 3M KOAc, pH 5.5, for example. In some embodiments, a cell lysis buffer is used in conjunction with the methods and components described herein.

Nucleic acid can be provided for conducting methods described herein without processing of the sample(s) containing the nucleic acid. For example, nucleic acid can be provided for conducting amplification methods described herein without prior nucleic acid purification. In some embodiments, a target sequence is amplified directly from a sample (e.g., without performing any nucleic acid extraction, isolation, purification and/or partial purification steps). In some embodiments, nucleic acid is provided for conducting methods described herein after processing of the sample(s) containing the nucleic acid. For example, a nucleic acid can be extracted, isolated, purified, or partially purified from the sample(s). The term “isolated” generally refers to nucleic acid removed from its original environment (e.g., the natural environment if it is naturally occurring, or a host cell if expressed exogenously), and thus is altered by human intervention (e.g., “by the hand of man”) from its original environment. The term “isolated nucleic acid” can refer to a nucleic acid removed from a subject (e.g., a human subject). An isolated nucleic acid can be provided with fewer non-nucleic acid components (e.g., protein, lipid, carbohydrate) than the amount of components present in a source sample. A composition comprising isolated nucleic acid can be about 50% to greater than 99% free of non-nucleic acid components. A composition comprising isolated nucleic acid can be about 90%, 91%, 92%, 93%, 94%, 95%, 96%, 97%, 98%, 99% or greater than 99% free of non-nucleic acid components. The term “purified” generally refers to a nucleic acid provided that contains fewer non-nucleic acid components (e.g., protein, lipid, carbohydrate) than the amount of non-nucleic acid components present prior to subjecting the nucleic acid to a purification procedure. A composition comprising purified nucleic acid may be about 80%, 81%, 82%, 83%, 84%, 85%, 86%, 87%, 88%, 89%, 90%, 91%, 92%, 93%, 94%, 95%, 96%, 97%, 98%, 99% or greater than 99% free of other non-nucleic acid components.

Nucleic acid may be provided for conducting methods described herein without modifying the nucleic acid. Modifications can include, for example, denaturation, digestion, nicking, unwinding, incorporation and/or ligation of heterogeneous sequences, addition of epigenetic modifications, addition of labels (e.g., radiolabels such as ³²P, ³³P, ¹²⁵I, or ³⁵S; enzyme labels such as alkaline phosphatase; fluorescent labels such as fluorescein isothiocyanate (FITC); or other labels such as biotin, avidin, digoxigenin, antigens, haptens, fluorochromes), and the like. Accordingly, in some embodiments, an unmodified nucleic acid is amplified.

Methods disclosed herein for detecting a target nucleic acid sequence (single-stranded or ds DNA and/or RNA) in a sample can detect a target nucleic acid sequence (e.g., DNA or RNA) with a high degree of sensitivity. In some embodiments, the method can be used to detect a target DNA/RNA present in a sample comprising a plurality of RNAs/DNAs (including the target RNA/DNA and a plurality of non-target RNAs/DNAs), wherein the target RNA/DNA is present at one or more copies per 10, 20, 25, 50, 100, 500, 10³, 5×10³, 10⁴, 5×10⁴, 10⁵, 5×10⁵, 10⁶, or 10⁷, non-target DNAs/RNAs. As used herein, the terms “RNA/DNA” and “RNAs/DNAs” shall be given their ordinary meaning, and shall also refer to DNA, or RNA, or a combination of DNA and RNA.

The threshold of detection, for a method of detecting a target RNA/DNA in a sample, can be, for example 10 nM or less. The term “threshold of detection” shall be given its ordinary meaning, and shall also describe the minimal amount of target RNA/DNA that must be present in a sample in order for detection to occur. As an illustrative example, when a threshold of detection is 10 nM, then a signal can be detected when a target RNA/DNA is present in the sample at a concentration of 10 nM or more. In some embodiments, a disclosed method has a threshold of detection of 5 nM or less, 1 nM or less, 0.5 nM or less, 0.1 nM or less, 0.05 nM or less, 0.01 nM or less, 0.005 nM or less, 0.001 nM or less, 0.0005 nM or less, 0.0001 nM or less, 0.00005 nM or less, 0.00001 nM or less, 10 pM or less, 1 pM or less, 500 fM or less, 250 fM or less, 100 fM or less, 50 fM or less, 500 aM (attomolar) or less, 250 aM or less, 100 aM or less, 50 aM or less, 10 aM or less, or 1 aM or less. The composition or method disclosed herein can have an attomolar (aM) sensitivity of detection, a femtomolar (fM) sensitivity of detection, a picomolar (pM) sensitivity of detection, or a nanomolar (nM) sensitivity of detection.

A sample can comprise sample nucleic acids (e.g., a plurality of sample nucleic acids). The term “plurality” is used herein to mean two or more. Thus, in some embodiments, a sample includes two or more (e.g., 3 or more, 5 or more, 10 or more, 20 or more, 50 or more, 100 or more, 500 or more, 1,000 or more, or 5,000 or more) sample nucleic acids (e.g., DNAs/RNAs). A disclosed method can be used as a very sensitive way to detect a target nucleic acid present in a sample (e.g., in a complex mixture of nucleic acids such as DNAs/RNAs). In some embodiments the sample includes 5, 10, 20, 25, 50, 100, 500, 10³, 5×10³, 10⁴, 5×10⁴, 10⁵, 5×10⁵, 10⁶, or 10⁷, 50, or more, DNAs/RNAs that differ from one another in sequence. In some embodiments, the sample includes DNAs/RNAs from a cell (e.g., a eukaryotic cell, a mammalian cell, or a human cell) or a cell lysate (e.g., a eukaryotic cell lysate, a mammalian cell lysate, a human cell lysate, a prokaryotic cell lysate, a plant cell lysate, or the like).

The term “sample” is used here shall be given its ordinary meaning and shall include any sample that includes RNA and/or DNA (e.g., in order to determine whether a target DNA and/or target RNA is present among a population of RNAs and/or DNAs). The sample can be derived from any source, e.g., the sample can be a synthetic combination of purified DNAs and/or RNAs; the sample can be a cell lysate, an DNA/RNA-enriched cell lysate, or DNAs/RNAs isolated and/or purified from a cell lysate. The sample can be from a patient (e.g., for the purpose of diagnosis). The sample can be from permeabilized cells, crosslinked cells, tissue sections, or combination thereof. The sample can be from tissues prepared by crosslinking followed by delipidation and adjustment to make a uniform refractive index. A sample can include a target nucleic acid (e.g., target DNA/RNA) and a plurality of non-target DNAs/RNAs. In some embodiments, the target DNA/RNA is present in the sample at one copy per 10, 20, 25, 50, 100, 500, 10³, 5×10³, 10⁴, 5×10⁴, 10⁵, 5×10⁵, 106, or 107, non-target DNAs/RNAs.

A sample with respect to a patient encompasses blood and other liquid samples of biological origin, solid tissue samples such as a biopsy specimen or tissue cultures or cells derived therefrom and the progeny thereof, as well as samples that have been manipulated in any way after their procurement (such as by treatment with reagents); washed; or enriched for certain cell populations (e.g., cancer cells) or particular types of molecules (e.g., RNAs). A sample can comprise, or be, a biological sample including but not limited to a clinical sample such as blood, plasma, serum, aspirate, cerebral spinal fluid (CSF), and also includes tissue obtained by surgical resection, tissue obtained by biopsy, cells in culture, cell supernatants, cell lysates, tissue samples, organs, bone marrow, and the like. A biological sample can comprise biological fluids derived therefrom (e.g., cancerous cell, infected cell, etc.), e.g., a sample comprising RNAs that is obtained from such cells (e.g., a cell lysate or other cell extract comprising RNAs).

The source of the sample can be a (or is suspected of being a) diseased cell, fluid, tissue, or organ; or a normal (non-diseased) cell, fluid, tissue, or organ. In some embodiments, the source of the sample is a (or is suspected of being a) pathogen-infected cell, tissue, or organ. For example, the source of a sample can be an individual who may or may not be infected—and the sample can be any biological sample (e.g., blood, saliva, biopsy, plasma, serum, bronchoalveolar lavage, sputum, a fecal sample, cerebrospinal fluid, a fine needle aspirate, a swab sample (e.g., a buccal swab, a cervical swab, a nasal swab), interstitial fluid, synovial fluid, nasal discharge, tears, buffy coat, a mucous membrane sample, an epithelial cell sample (e.g., epithelial cell scraping), etc.) collected from the individual. The sample can be a cell-free liquid sample or a liquid sample that comprise cells. Pathogens can be viruses, fungi, helminths, protozoa, malarial parasites, Plasmodium parasites, Toxoplasma parasites, Schistosoma parasites, and the like. “Helminths” include roundworms, heartworms, and phytophagous nematodes (Nematoda), flukes (Tematoda), Acanthocephala, and tapeworms (Cestoda). Protozoan infections include infections from Giardia spp., Trichomonas spp., African trypanosomiasis, amoebic dysentery, babesiosis, balantidial dysentery, Chaga's disease, coccidiosis, malaria and toxoplasmosis. Examples of pathogens such as parasitic/protozoan pathogens include, but are not limited to: Plasmodium falciparum, Plasmodium vivax, Trypanosoma cruzi and Toxoplasma gondii. Fungal pathogens include, but are not limited to: Cryptococcus neoformans, Histoplasma capsulatum, Coccidioides immitis, Blastomyces dermatitidis, Chlamydia trachomatis, and Candida albicans. Pathogenic viruses include, e.g., immunodeficiency virus (e.g., HIV); influenza virus; dengue; West Nile virus; herpes virus; yellow fever virus; Hepatitis C virus; Hepatitis A virus; Hepatitis B virus; papillomavirus; and the like. Pathogenic viruses can include DNA viruses such as: a papovavirus (e.g., HPV, polyomavirus); a hepadnavirus; a herpesvirus (e.g., HSV (e.g., HSV I, HSV II), varicella zoster virus (VZV), epstein-barr virus (EBV), cytomegalovirus (CMV), herpes lymphotropic virus, Pityriasis Rosea, kaposi's sarcoma-associated herpesvirus); an adenovirus (e.g., atadenovirus, aviadenovirus, ichtadenovirus, mastadenovirus, siadenovirus); a poxvirus (e.g., smallpox, vaccinia virus, cowpox virus, monkeypox virus, orf virus, pseudocowpox, bovine papular stomatitis virus; tanapox virus, yaba monkey tumor virus; molluscum contagiosum virus (MCV)); a parvovirus (e.g., adeno-associated virus (AAV), Parvovirus B19, human bocavirus, bufavirus, human parv4 G1); Geminiviridae; Nanoviridae; Phycodnaviridae; and the like. Non-limiting examples of pathogens include Mycobacterium tuberculosis, Streptococcus agalactiae, methicillin-resistant Staphylococcus aureus, Legionella pneumophila, Streptococcus pyogenes, Escherichia coli, Neisseria gonorrhoeae, Neisseria meningitidis, Pneumococcus, Cryptococcus neoformans, Histoplasma capsulatum, Hemophilus influenzae B, Treponema pallidum, Lyme disease spirochetes, Pseudomonas aeruginosa, Mycobacterium leprae, Brucella abortus, rabies virus, human serum parvo-like virus, respiratory syncytial virus, measles virus, adenovirus, human T-cell leukemia viruses, murine leukemia virus, mumps virus, vesicular stomatitis virus, Sindbis virus, lymphocytic choriomeningitis virus, wart virus, blue tongue virus, Sendai virus, feline leukemia virus, reovirus, polio virus, simian virus 40, mouse mammary tumor virus, dengue virus, rubella virus, Toxoplasma gondii, Trypanosoma rangeli, Trypanosoma cruzi, Trypanosoma rhodesiense, Trypanosoma brucei, Schistosoma mansoni, Schistosoma japonicum, Babesia bovis, Eimeria tenella, Onchocerca volvulus, Leishmania tropica, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium and M. pneumoniae.

### Amplification

Provided herein include methods for amplifying nucleic acid. In some embodiments, nucleic acids are amplified using a suitable amplification process. Nucleic acid amplification involves enzymatic synthesis of nucleic acid amplicons (copies), which contain a sequence complementary to a nucleotide sequence being amplified. The amplification cam be performed in a single vessel, a single chamber, and/or a single volume (i.e., contiguous volume). In some embodiments, the amplification and detection (e.g., a detection method described herein) are performed in a single vessel, a single chamber, and/or a single volume (i.e., contiguous volume).

The terms “amplify”, “amplification”, “amplification reaction”, or “amplifying” refer to any in vitro process for multiplying the copies of a target nucleic acid. Amplification sometimes refers to an “exponential” increase in target nucleic acid. “Amplifying” can also refer to linear increases in the numbers of a target nucleic acid, but is different than a one-time, single primer extension step. In some embodiments a limited amplification reaction, also known as pre-amplification, can be performed. Pre-amplification is a method in which a limited amount of amplification occurs due to a small number of cycles, for example 10 cycles, being performed. Pre-amplification can allow some amplification, but stops amplification prior to the exponential phase, and typically produces about 500 copies of the desired nucleotide sequence(s). Use of pre-amplification may limit inaccuracies associated with depleted reactants in certain amplification reactions, and also may reduce amplification biases due to nucleotide sequence or species abundance of the target. In some embodiments a one-time primer extension may be performed as a prelude to linear or exponential amplification.

A generalized description of an amplification process is presented herein. Primers (e.g., oligonucleotides described herein) and target nucleic acid are contacted, and complementary sequences anneal or hybridize to one another, for example. Primers can anneal to a target nucleic acid, at or near (e.g., adjacent to, abutting, and the like) a sequence of interest. A primer annealed to a target may be referred to as a primer-target hybrid, hybridized primer-target, or a primer-target duplex. The terms near or adjacent to when referring to a nucleotide sequence of interest refer to a distance (e.g., number of bases) or region between the end of the primer and the nucleotide or nucleotides (e.g., nucleotide sequence) of a target. Generally, adjacent is in the range of about 1 nucleotide to about 50 nucleotides (e.g., about 1, 2, 3, 4, 5, 6, 7, 8, 9, 10, 20, 30, 40, or 50 nucleotide(s)) away from a nucleotide or nucleotide sequence of interest. In some embodiments, primers in a set (e.g., a pair of primers, a forward and a reverse primer, a first oligonucleotide and a second oligonucleotide) anneal within about 1 to 20 nucleotides from a nucleotide or nucleotide sequence of interest and produce amplified products. In some embodiments, primers anneal within a nucleotide or a nucleotide sequence of interest. After annealing, each primer is extended along the target (i.e., template strand) by a polymerase to generate a complementary strand. Several cycles of primer annealing and extension can be carried out, for example, until a detectable amount of amplification product is generated. In some embodiments, where a target nucleic acid is RNA, a DNA copy (cDNA) of the target RNA is synthesized prior to or during the amplification step by reverse transcription.

Components of an amplification reaction (e.g., the one or more amplification reagents) can include, for example, one or more primers (e.g., individual primers, primer pairs, primer sets, oligonucleotides, multiple primer sets for multiplex amplification, and the like), nucleic acid target(s) (e.g., target nucleic acid from a sample), one or more polymerases, nucleotides (e.g., dNTPs and the like), and a suitable buffer (e.g., a buffer comprising a detergent, a reducing agent, monovalent ions, and divalent ions). An amplification reaction can further include one or more of: a reverse transcriptase, a reverse transcription primer, and one or more detection agents.

Nucleic acid amplification can be conducted in the presence of native nucleotides, for example, dideoxyribonucleoside triphosphates (dNTPs), and/or derivatized nucleotides. A native nucleotide generally refers to adenylic acid, guanylic acid, cytidylic acid, thymidylic acid, or uridylic acid. A derivatized nucleotide generally is a nucleotide other than a native nucleotide. A ribonucleoside triphosphate is referred to as NTP or rNTP, where N can be A, G, C, U. A deoxynucleoside triphosphate substrates is referred to as dNTP, where N can be A, G, C, T, or U. Monomeric nucleotide subunits may be denoted as A, G, C, T, or U herein with no particular reference to DNA or RNA. In some embodiments, non-naturally occurring nucleotides or nucleotide analogs, such as analogs containing a detectable label (e.g., fluorescent or colorimetric label), may be used. For example, nucleic acid amplification can be carried out in the presence of labeled dNTPs, for example, radiolabels such as ³²P, ³³P, ¹²⁵I, or ³⁵S; enzyme labels such as alkaline phosphatase; fluorescent labels such as fluorescein isothiocyanate (FITC); or other labels such as biotin, avidin, digoxigenin, antigens, haptens, or fluorochromes. In some embodiments, nucleic acid amplification may be carried out in the presence of modified dNTPs, for example, heat activated dNTPs (e.g., CleanAmp™ dNTPs from TriLink).

The one or more amplification reagents can include non-enzymatic components and enzymatic components. Non-enzymatic components can include, for example, primers, nucleotides, buffers, salts, reducing agents, detergents, and ions. In some embodiments, the Non-enzymatic components do not include proteins (e.g., nucleic acid binding proteins), enzymes, or proteins having enzymatic activity, for example, polymerases, reverse transcriptases, helicases, topoisomerases, ligases, exonucleases, endonucleases, restriction enzymes, nicking enzymes, recombinases and the like. In some embodiments, an enzymatic component consists of a polymerase or consists of a polymerase and a reverse transcriptase. Accordingly, such enzymatic components would exclude other proteins (e.g., nucleic acid binding proteins and/or proteins having enzymatic activity), for example, helicases, topoisomerases, ligases, exonucleases, endonucleases, restriction enzymes, nicking enzymes, recombinases, and the like.

In some embodiments, amplification conditions comprise an enzymatic activity (e.g., an enzymatic activity provided by a polymerase or provided by a polymerase and a reverse transcriptase). In some embodiments, the enzymatic activity does not include enzymatic activity provided by enzymes other than the polymerase and/or the reverse transcriptase, for example, helicases, topoisomerases, ligases, exonucleases, endonucleases, restriction enzymes, nicking enzymes, recombinases, and the like. A polymerase activity and a reverse transcriptase activity can be provided by separate enzymes or separate enzyme types (e.g., polymerase(s) and reverse transcriptase(s)), or provided by a single enzyme or enzyme type (e.g., polymerase(s)).

Amplification of nucleic acid can comprise a non-thermocycling type of PCR. In some embodiments, amplification of nucleic acid comprises an isothermal amplification process, for example an isothermal polymerase chain reaction (iPCR). Isothermal amplification generally is an amplification process performed at a constant temperature. Terms such as isothermal conditions, isothermally and constant temperature generally refer to reaction conditions where the temperature of the reaction is kept essentially constant during the course of the amplification reaction. Isothermal amplification conditions generally do not include a thermocycling (i.e., cycling between an upper temperature and a lower temperature) component in the amplification process. When amplifying under isothermal conditions, the reaction can be kept at an essentially constant temperature, which means the temperature may not be maintained at precisely one temperature. For example, small fluctuations in temperature (e.g., +1 to 5° C.) may occur in an isothermal amplification process due to, for example, environmental or equipment-based variables. Often, the entire reaction volume is kept at an essentially constant temperature, and isothermal reactions herein generally do not include amplification conditions that rely on a temperature gradient generated within a reaction vessel and/or convective-flow based temperature cycling.

Isothermal amplification reactions herein can be conducted at an essentially constant temperature. In some embodiments, isothermal amplification reactions herein are conducted at a temperature of about 55° C. to a temperature of about 75° C., for example at a temperature of, or a temperature of about, 55, 56, 57, 58, 59, 60, 61, 62, 63, 64, 65, 66, 67, 68, 69, 70, 71, 72, 73, 74, or about 75° C., or a number or a range between any two of these values. In some embodiments, a temperature element (e.g., heat source) is kept at an essentially constant temperature, for example an essentially constant temperature at or below about 75° C., at or below about 70° C., at or below about 65° C., or at or below about 60° C.

An amplification process herein can be conducted over a certain length of time, for example until a detectable nucleic acid amplification product is generated. A nucleic acid amplification product may be detected by any suitable detection process and/or a detection process described herein. The amplification process can be conducted over a length of time within about 20 minutes or less, or about 10 minutes or less. For example, an amplification process can be conducted within about 1, 2, 3, 4, 5, 6, 7, 8, 9, 10, 11, 12, 13, 14, 15, 16, 17, 18, 19 or 20 minutes, or a number or a range between any two of these values.

Nucleic acid targets can be amplified without exposure to agents or conditions that denature nucleic acid, in some embodiments. Nucleic acid targets can be amplified without exposure to agents or conditions that promote strand separation during the amplification step (and/or other steps) in some embodiments. Nucleic acid targets can be amplified without exposure to agents or conditions that promote unwinding during the amplification step (and/or other steps) in some embodiments. Agents or conditions that denature nucleic acid and/or promote strand separation and/or promote unwinding may include, for example, thermal conditions (e.g., high temperatures), pH conditions (e.g., high or low pH), chemical agents, proteins (e.g., enzymatic agents), and the like.

In some embodiments, the methods disclosed herein does not comprise thermal denaturation (e.g., heating a solution containing a nucleic acid to an elevated temperature, such as, for example a temperature above 75° C., 80° C., 90° C., or 95° C., or higher) or protein-based (e.g., enzymatic) denaturation of a nucleic acid. Protein-based (e.g., enzymatic) denaturation can comprise contacting a nucleic acid with one or more of a helicase, a topoisomerase, a ligase, an exonuclease, an endonuclease, a restriction enzyme, a nicking enzyme, a recombinase, a RNA replicase, and a nucleic acid binding protein (e.g., single-stranded binding protein). In some embodiments, the compositions provided herein do not comprise a helicase, a topoisomerase, a ligase, an exonuclease, an endonuclease, a restriction enzyme, a nicking enzyme, a recombinase, a RNA replicase, and/or a nucleic acid binding protein (e.g., single-stranded binding protein). In some embodiments, the compositions and methods provided herein do not comprise intercalators, alkylating agents, and/or chemicals such as formamide, glycerol, urea, dimethyl sulfoxide (DMSO), or N,N,N-trimethylglycine (betaine). In some embodiments, the disclosed methods do not comprise contacting a nucleic acid with denaturing agents (e.g., formamide). In some embodiments, the amplifying step does not comprise agents and/or conditions that denature nucleic acids (e.g., promote strand separation and/or promote unwinding). In some embodiments, the amplifying step (e.g., step (c)) does not comprise agents and/or conditions that denature nucleic acids (e.g., promote strand separation and/or promote unwinding) other than a polymerase (e.g., a hyperthermophile polymerase). In some embodiments, the methods and compositions provided herein not comprise agents and/or conditions that denature nucleic acids (e.g., promote strand separation and/or promote unwinding) other than a polymerase (e.g., a hyperthermophile polymerase) and/or low pH conditions (e.g., contact with acid(s)).

Nucleic acid targets can be amplified without exposure to agents or conditions that promote strand separation and/or unwinding, for example a helicase, a topoisomerase, a ligase, an exonuclease, an endonuclease, a restriction enzyme, a nicking enzyme, a recombinase, a RNA replicase, a nucleic acid binding protein (e.g., single-stranded binding protein), or any combination thereof. For example, nucleic acid targets can be amplified without exposure to a helicase, including but not limited to DNA helicases and RNA helicases. Amplification conditions that do not include use of a helicase are helicase-free amplification conditions.

Nucleic acid targets can be amplified without exposure to a recombinase, including but not limited to, Cre recombinase, Hin recombinase, Tre recombinase, FLP recombinase, RecA, RAD51, RadA, T4 uvsX. In some embodiments, nucleic acid targets are amplified without exposure to a recombinase accessory protein, for example, a recombinase loading factor (e.g., T4 uvsY). Nucleic acid targets can be amplified without exposure to a nucleic acid binding protein (e.g., single-stranded binding protein or single-strand DNA-binding protein (SSB)), for example, T4 gp32. In some embodiments, nucleic acid targets are amplified without exposure to a topoisomerase. Nucleic acid targets can be amplified with or without exposure to agents or conditions that destabilize nucleic acid. As used herein, the term “destabilization” shall be given its ordinary meaning, and shall also refer to a disruption in the overall organization and geometric orientation of a nucleic acid molecule (e.g., double helical structure) by one or more of tilt, roll, twist, slip, and flip effects (e.g., as described in Lenglet et al., (2010) Journal of Nucleic Acids Volume 2010, Article ID 290935, 17 pages). Destabilization generally does not refer to melting or separation of nucleic acid strands (e.g., denaturation). Nucleic acid destabilization can be achieved, for example, by exposure to agents such as intercalators or alkylating agents, and/or chemicals such as formamide, urea, DMSO, or betaine. In some embodiments, methods provided herein include use of one or more destabilizing agents. In some embodiments, methods provided herein exclude use of destabilizing agents. In some embodiments, nucleic acid targets are amplified without exposure to a ligase and/or an RNA replicase.

Nucleic acid targets can be amplified without cleavage or digestion, in some embodiments. For example, nucleic acid targets can be amplified without prior exposure to one or more cleavage agents, and intact nucleic acid is amplified. In some embodiments, nucleic acid targets are amplified without exposure to one or more cleavage agents during amplification. In some embodiments, nucleic acid targets are amplified without exposure to one or more cleavage agents after amplification. Amplification conditions that do not include use of a cleavage agent may be referred to herein as cleavage agent-free amplification conditions. The term “cleavage agent” generally refers to an agent, sometimes a chemical or an enzyme that can cleave a nucleic acid at one or more specific or non-specific sites. Specific cleavage agents often cleave specifically according to a particular nucleotide sequence at a particular site. Cleavage agents can include endonucleases (e.g., restriction enzymes, nicking enzymes, and the like); exonucleases (DNAses, RNAses (e.g., RNAseH), 5′ to 3′ exonucleases (e.g. exonuclease II), 3′ to 5′ exonucleases (e.g. exonuclease I), and poly(A)-specific 3′ to 5′ exonucleases); and chemical cleavage agents.

Nucleic acid targets can be amplified without use of restriction enzymes and/or nicking enzymes. In some embodiments, nucleic acid is amplified without prior exposure to restriction enzymes and/or nicking enzymes. In some embodiments, nucleic acid is amplified without exposure to restriction enzymes and/or nicking enzymes during amplification. In some embodiments, nucleic acid is amplified without exposure to restriction enzymes and/or nicking enzymes after amplification. Nucleic acid targets can be amplified without exonuclease treatment. Exonucleases include, for example, DNAses, RNAses (e.g., RNAseH), 5′ to 3′ exonucleases (e.g. exonuclease II), 3′ to 5′ exonucleases (e.g. exonuclease I), and poly(A)-specific 3′ to 5′ exonucleases. In some embodiments, nucleic acid is amplified without exonuclease treatment prior to, during, and/or after amplification. Amplification conditions that do not include use of an exonuclease are exonuclease-free amplification conditions. In some embodiments, nucleic acid is amplified without DNAse treatment and/or RNAse treatment.

An amplified nucleic acid may be referred to herein as a nucleic acid amplification product or amplicon. In some embodiments, the amplification product includes naturally occurring nucleotides, non-naturally occurring nucleotides, nucleotide analogs and the like and combinations of the foregoing. An amplification product typically has a nucleotide sequence that is identical to or substantially identical to a sequence in a sample nucleic acid (e.g., target sequence) or complement thereof. A “substantially identical” nucleotide sequence in an amplification product will generally have a high degree of sequence identity to the nucleotide sequence being amplified or complement thereof (e.g., about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or greater than 99% sequence identity), and variations sometimes are a result of polymerase infidelity or other variables.

In some embodiments, a nucleic acid amplification product comprises a polynucleotide that is continuously complementary to or substantially identical to a target sequence in sample nucleic acid. Continuously complementary generally refers to a nucleotide sequence in a first strand, for example, where each base in order (e.g., read 5′ to 3′) pairs with a correspondingly ordered base in a second strand, and there are no gaps, additional sequences or unpaired bases within the sequence considered as continuously complementary. Stated another way, continuously complementary generally refers to all contiguous bases of a nucleotide sequence in a first stand being complementary to corresponding contiguous bases of a nucleotide sequence in a second strand. For example, a first strand having a sequence 5′-ATGCATGCATGC-3′ (SEQ ID NO: 3) would be considered as continuously complementary to a second strand having a sequence 5′-GCATGCATGCAT-3′ (SEQ ID NO: 4), where all contiguous bases in the first strand are complementary to all corresponding contiguous bases in the second strand. However, a first strand having a sequence 5′-ATGCATAAAAAAGCATGC-3′ (SEQ ID NO: 5) would not be considered as continuously complementary to a second strand having a sequence 5′-GCATGCATGCAT-3′ (SEQ ID NO: 4), because the sequence of six adenines (6 As) in the middle of the first strand would not pair with bases in the second strand. A continuously complementary sequence sometimes is about 5 to about 25 contiguous bases in length, e.g., about 5, 6, 7, 8, 9, 10, 11, 12, 13, 14, 15, 16, 17, 18, 19, 20, 21, 22, 23, 24, 25, or a range between any two of these values, contiguous bases in length. In some embodiments, a nucleic acid amplification product consists of a polynucleotide that is continuously complementary to or substantially identical to a target sequence in sample nucleic acid. Accordingly, in some embodiments, a nucleic acid amplification product does not include any additional sequences (e.g., at the 5′ and/or 3′ end, or within the product) that are not continuously complementary to or substantially identical to a target sequence, for example, additional sequences incorporated into an amplification product by way of tailed primers or ligation, and/or additional sequences providing cleavage agent recognition sites (e.g., nicking enzyme recognition sites). Generally, unless a target sequence comprises tandem repeats, an amplification product does not include product in the form of tandem repeats.

Nucleic acid amplification products can comprise sequences complementary to or substantially identical to one or more primers used in an amplification reaction. In some embodiments, a nucleic acid amplification product comprises a first nucleotide sequence that is continuously complementary to or identical to a first primer sequence, and a second nucleotide sequence that is continuously complementary to or identical to a second primer sequence.

Nucleic acid amplification products can comprise a spacer sequence. As described herein, a spacer sequence in an amplification product is a sequence (1 or more bases) continuously complementary to or substantially identical to a portion of a target sequence in the sample nucleic acid, and is flanked by sequences in the amplification product that are complementary to or substantially identical to one or more primers used in an amplification reaction. A spacer sequence flanked by sequences in the amplification product generally lies between a first sequence (complementary to or substantially identical to a first primer) and a second sequence (complementary to or substantially identical to a second primer). Thus, an amplification product typically includes a first sequence followed by a spacer sequences followed by a second sequence. A spacer sequence generally is not complementary to or substantially identical to a sequence in the primer(s). A spacer sequence can be, or can comprise, about 1 to 10 bases, including 1, 2, 3, 4, 5, 6, 7, 8, 9, or 10 bases. In some embodiments, a nucleic acid amplification product consists of, or consists essentially of, a first nucleotide sequence that is continuously complementary to or identical to a first primer sequence, a second nucleotide sequence that is continuously complementary to or identical to a second primer sequence, and a spacer sequence. In some embodiments, a nucleic acid amplification product does not include any additional sequences (e.g., at the 5′ and/or 3′ end; or within the product) that are not continuously complementary to or identical to a first primer sequence and a second primer sequence, and are not part of a spacer sequence, for example, additional sequences incorporated into an amplification product by way of tailed or looped primers, ligation or other mechanism. In some embodiments, a nucleic acid amplification product generally does not include additional sequences (e.g., at the 5′ and/or 3′ end; or within the product) that are not continuously complementary to or identical to a first primer sequence and a second primer sequence, and are not part of a spacer sequence, for example, additional sequences incorporated into an amplification product by way of tailed or looped primers, ligation or other mechanism. However, in such embodiments, a nucleic acid amplification product may include, for example, some mismatched (i.e., non-complementary) bases or one more extra bases (e.g., at the 5′ and/or 3′ end; or within the product) introduced into the product by way of error or promiscuity in the amplification process.

Nucleic acid amplification products can be up to 50 bases in length, including 10, 15, 20, 25, 30, 35, 40, 45, 50, or a number or a range between any two of these values, bases long. In some embodiments, nucleic acid amplification products for a given target sequence have the same length or substantially the same length (e.g., within 1 to 10 bases). Accordingly, nucleic acid amplification products for a given target sequence may produce a single signal (e.g., band on an electrophoresis gel) and generally do not produce multiple signals indicative of multiple lengths (e.g., a ladder or smear on an electrophoresis gel). For multiplex reactions, nucleic acid amplification products for different target sequences may have different lengths.

The methods and components described herein can be used for multiplex amplification which generally refers to the amplification of more than one nucleic acid of interest (e.g., amplification or more than one target sequence). For example, multiplex amplification can refer to amplification of multiple sequences from the same sample or amplification of one of several sequences in a sample. Multiplex amplification also can refer to amplification of one or more sequences present in multiple samples either simultaneously or in step-wise fashion. For example, a multiplex amplification can be used for amplifying least two target sequences that are capable of being amplified (e.g., the amplification reaction comprises the appropriate primers and enzymes to amplify at least two target sequences). In some embodiments, an amplification reaction is prepared to detect at least two target sequences, but only one of the target sequences is present in the sample being tested, such that both sequences are capable of being amplified, but only one sequence is amplified. In some embodiments, where two target sequences are present, an amplification reaction results in the amplification of both target sequences. A multiplex amplification reaction can result in the amplification of one, some, or all of the target sequences for which it comprises the appropriate primers and enzymes. In some embodiments, an amplification reaction is prepared to detect two sequences with one pair of primers, where one sequence is a target sequence and one sequence is a control sequence (e.g., a synthetic sequence capable of being amplified by the same primers as the target sequence and having a different spacer base or sequence than the target). In some embodiments, an amplification reaction is prepared to detect multiple sets of sequences with corresponding primer pairs, where each set includes a target sequence and a control sequence.

### Primers

Nucleic acid amplification generally is conducted in the presence of one or more primers. A primer is generally characterized as an oligonucleotide that includes a nucleotide sequence capable of hybridizing or annealing to a target nucleic acid, at or near (e.g., adjacent to) a specific region of interest (i.e., target sequence). Primers can allow for specific determination of a target nucleic acid nucleotide sequence or detection of the target nucleic acid (e.g., presence or absence of a sequence), or feature thereof, for example. A primer can be naturally occurring or synthetic. The term specific, or specificity, generally refers to the binding or hybridization of one molecule to another molecule, such as a primer for a target polynucleotide. That is, specific or specificity refers to the recognition, contact, and formation of a stable complex between two molecules, as compared to substantially less recognition, contact, or complex formation of either of those two molecules with other molecules. The term anneal or hybridize generally refers to the formation of a stable complex between two molecules. The terms primer, oligo, or oligonucleotide may be used interchangeably herein, when referring to primers.

A primer can be designed and synthesized using suitable processes, and can be of any length suitable for hybridizing to a target sequence and performing an amplification process described herein. Primers often are designed according to a sequence in a target nucleic acid. A primer in some embodiments may be about 5 to about 30 bases in length, for example 5, 6, 7, 8, 9, 10, 11, 12, 13, 14, 15, 16, 17, 18, 19, 20, 21, 22, 23, 24, 25, 26, 27, 28, 29 or 30 bases in length. A primer may be composed of naturally occurring and/or non-naturally occurring nucleotides (e.g., modified nucleotides, labeled nucleotides), or a mixture thereof. Modifications and modified bases may include, for example, phosphorylation, (e.g., 3′ phosphorylation, 5′ phosphorylation); attachment chemistry or linkers modifications (e.g., Acrydite™, adenylation, azide (NHS ester), digoxigenin (NHS ester), cholesteryl-TEG, I-Linker™, amino modifiers (e.g., amino modifier C6, amino modifier C12, amino modifier C6 dT, Uni-Link™ amino modifier), alkynes (e.g., 5′ hexynyl, 5-octadiynyl dU), biotinylation (e.g., biotin, biotin (azide), biotin dT, biotin-TEG, dual biotin, PC biotin, desthiobiotin-TEG), thiol modifications (e.g., thiol modifier C3 S-S, dithiol, thiol modifier C6 S-S)); fluorophores (e.g., Freedom™ Dyes, Alexa Fluor® Dyes, LI-COR IRDyes®, ATTO™ Dyes, Rhodamine Dyes, WellRED Dyes, 6-FAM (azide), Texas Red®-X (NHS ester), Lightcycler® 640 (NHS ester), Dy 750 (NHS ester)); Iowa Black® dark quenchers modifications (e.g., lowa Black® FQ, Iowa Black® RQ); dark quenchers modifications (e.g., Black Hole Quencher®-1, Black Hole Quencher®-2, Dabcyl); spacers (C3 spacer, PC spacer, hexanediol, spacer 9, spacer 18, 1′,2′-dideoxyribose (dSpacer); modified bases (e.g., 2-aminopurine, 2,6-diaminopurine (2-amino-dA), 5-bromo dU, deoxyUridine, inverted dT, inverted dideoxy-T, dideoxy-C, 5-methyl dC, deoxyInosine, Super TR, Super G®, locked nucleic acids (LNA's), 5-nitroindole, 2′-O-methyl RNA bases, hydroxmethyl dC, UNA unlocked nucleic acid (e.g., UNA-A, UNA-U, UNA-C, UNA-G), Iso-dC, Iso-dG, Fluoro C, Fluoro U, Fluoro A, Fluoro G); phosphorothioate bonds modifications (e.g., phosphorothioated DNA bases, phosphorothioated RNA bases, phosphorothioated 2′ O-methyl bases, phosphorothioated LNA bases); and click chemistry modifications. In some embodiments, modifications and modified bases include uracil bases, ribonucleotide bases, O-methyl RNA bases, phosphorothioate linkages, 3′ phosphate groups, spacer bases (such as C3 spacer or other spacer bases). For example, a primer may comprise one or more O-methyl RNA bases (e.g., 2′-O-methyl RNA bases). 2′-O-methyl RNA generally is a post-transcriptional modification of RNA found in tRNA and other small RNAs. Primers can be directly synthesized that include 2′-O-methyl RNA bases. This modification can, for example, increase Tm of RNA: RNA duplexes and provide stability in the presence of single-stranded ribonucleases and DNases. 2′-O-methyl RNA bases may be included in primers, for example, to increase stability and binding affinity to a target sequence. In some embodiments, a primer may comprise one or more phosphorothioate linkages (e.g., phosphorothioate bond modifications). A phosphorothioate (PS) bond substitutes a sulfur atom for a non-bridging oxygen in the phosphate backbone of a primer. This modification typically renders the internucleotide linkage resistant to nuclease degradation. Phosphorothioate bonds may be introduced between about the last 3 to 5 nucleotides at the 5′-end or the 3′-end of a primer to inhibit exonuclease degradation, for example. Phosphorothioate bonds included throughout an entire primer can help reduce attack by endonucleases, in some embodiments. A primer can, for example, comprise a 3′ phosphate group. 3′ phosphorylation can inhibit degradation by certain 3′-exonucleases and can be used to block extension by DNA polymerases, in certain instances. In some embodiments, a primer comprises one or more spacer bases (e.g., one or more C3 spacers). A C3 spacer phosphoramidite can be incorporated internally or at the 5′-end of a primer. Multiple C3 spacers can be added at either end of a primer to introduce a long hydrophilic spacer arm for the attachment of fluorophores or other pendent groups, for example.

A primer can comprises DNA bases, RNA bases, or both, where one or more of the DNA bases and RNA bases is modified or unmodified. For example, a primer can be a mixture of DNA bases and RNA bases. The primer can consist of DNA bases (e.g., modified DNA bases and/or unmodified DNA bases). In some embodiments, the primer consists of unmodified DNA bases. In some embodiments, the primer consists of modified DNA bases. The primer can consist of RNA bases (e.g., modified RNA bases and/or unmodified RNA bases). In some embodiments, the primer consists of unmodified RNA bases. In some embodiments, the primer consists of modified RNA bases. In some embodiments, a primer comprises no RNA bases. In some embodiments, a primer comprises no DNA bases. In some embodiments, the primer comprises no cleavage agent recognition sites (e.g., no nicking enzyme recognition sites). In some embodiments, a primer comprises no tail (e.g., no tail comprising a nicking enzyme recognition site).

All or a portion of a primer sequence can be complementary or substantially complementary to a target nucleic acid, in some embodiments. Substantially complementary with respect to sequences generally refers to nucleotide sequences that will hybridize with each other. The stringency of the hybridization conditions can be altered to tolerate varying amounts of sequence mismatch. The target and primer sequences can be, for example, at least 75% complementary to each other, including 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% complementary to each other. Primers that are substantially complimentary to a target nucleic acid sequence typically are also substantially identical to the complement of the target nucleic acid sequence (i.e., the sequence of the anti-sense strand of the target nucleic acid). The primer and the anti-sense strand of the target nucleic acid can be at least 75% identical in sequence, for example 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% identical to each other.

In some embodiments, primers comprise a pair of primers. A pair of primers may include a forward primer and a reverse primer (e.g., primers that bind to the sense and antisense strands of a target nucleic acid). In some embodiments, primers consist of a pair of primers (i.e. a forward primer and a reverse primer). Accordingly, in some embodiments, amplification of a target sequence is performed using a pair of primers and no additional primers or oligonucleotides are included in the amplification of the target sequence (e.g., the amplification reaction components comprise no additional primer pairs for a given target sequence, no nested primers, no bumper primers, no oligonucleotides other than the primers, no probes, and the like). In some embodiments, primers consist of a pair of primers. In some embodiments, an amplification reaction can include additional primer pairs for amplifying different target sequences, such as in a multiplex amplification. In some embodiments, primers consist of a pair of primers, however, in some embodiments, an amplification reaction can include additional primers, oligonucleotides or probes for a detection process that are not considered part of amplification. In some embodiments, primers are used in sets. An amplification primer set can include a pair of forward and reverse primers for a given target sequence. For multiplex amplification, primers that amplify a first target sequence are considered a primer set, and primers that amplify a second target sequence are considered a different primer set.

Amplification reaction components can comprise, or consist of, a first primer (first oligonucleotide) complementary to a target sequence in a first strand (e.g., sense strand, forward strand) of a sample nucleic acid, and a second primer (second oligonucleotide) complementary to a target sequence in a second strand (e.g., antisense strand, reverse strand) of a sample nucleic acid. In some embodiments, a first primer (first oligonucleotide) comprises a first polynucleotide continuously complementary to a target sequence in a first strand of sample nucleic acid, and a second primer (second oligonucleotide) comprises a second polynucleotide continuously complementary to a target sequence in a second strand of sample nucleic acid. Continuously complementary for a primer-target generally refers to a nucleotide sequence in a primer, where each base in order pairs with a correspondingly ordered base in a target sequence, and there are no gaps, additional sequences or unpaired bases within the sequence considered as continuously complementary. In some embodiments, a primer does not include any additional sequences (e.g., at the 5′ and/or 3′ end, or within the primer) that are not continuously complementary to a target sequence, for example, additional sequences present in tailed primers or looped primers, and/or additional sequences providing cleavage agent recognition sites (e.g., nicking enzyme recognition sites). In some embodiments, amplification reaction components do not comprise primers comprising additional sequences (i.e., sequences other than the sequence that is continuously complementary to a target sequence), for example, tailed primers, looped primers, primers capable of forming step-loop structures, hairpin structures, and/or additional sequences providing cleavage agent recognition sites (e.g., nicking enzyme recognition sites), and the like.

The primer, in some embodiments, can contain a modification such as one or more inosines, abasic sites, locked nucleic acids, minor groove binders, duplex stabilizers (e.g., acridine, spermidine), Tm modifiers or any modifier that changes the binding properties of the primer. The primer, in some embodiments, can contain a detectable molecule or entity (e.g., a fluorophore, radioisotope, colorimetric agent, particle, enzyme and the like).

### Polymerase

In some embodiments, amplification reaction components (e.g., one or more amplification reagents) comprise one or more polymerases. Polymerases are proteins capable of catalyzing the specific incorporation of nucleotides to extend a 3′ hydroxyl terminus of a primer molecule, for example, an amplification primer described herein, against a nucleic acid target sequence (e.g., to which a primer is annealed). Non-limiting examples of polymerases include thermophilic or hyperthermophilic polymerases that can have activity at an elevated reaction temperature (e.g., above 55, 60, 65, 70, 75, 80, 85, 90, 95, or 100° C.). A hyperthermophilic polymerase may be referred to as a hyperthermophile polymerase. A polymerase may or may not have strand displacement capabilities. In some embodiments, a polymerase can incorporate about 1 to about 50 nucleotides in a single synthesis, for example about 1, 5, 10, 15, 20, 25, 30, 35, 40, 45 or 50 nucleotides, or a number or a range between any two of these values, in a single synthesis.

The amplification reaction components can comprise one or more DNA polymerases selected from: 9°N DNA polymerase; 9°Nm™ DNA polymerase; Therminator™ DNA Polymerase; Therminator™ II DNA Polymerase; Therminator™ III DNA Polymerase; Therminator™ y DNA Polymerase; Bst DNA polymerase; Bst DNA polymerase (large fragment); Phi29 DNA polymerase, DNA polymerase I (E. coli), DNA polymerase I, large (Klenow) fragment; Klenow fragment (3′-5′ exo-); T4 DNA polymerase; T7 DNA polymerase; Deep VentR™ (exo-) DNA Polymerase; Deep VentR™ DNA Polymerase; DyNAzyme™ EXT DNA; DyNAzyme™ II Hot Start DNA Polymerase; Phusion™ High-Fidelity DNA Polymerase; VentR® DNA Polymerase; VentR® (exo-) DNA Polymerase; RepliPHI™ Phi29 DNA Polymerase; rBst DNA Polymerase, large fragment (IsoTherm™ DNA Polymerase); MasterAmp™ AmpliTherm™ DNA Polymerase; Tag DNA polymerase; Tth DNA polymerase; Tfl DNA polymerase; Tgo DNA polymerase; SP6 DNA polymerase; Tbr DNA polymerase; DNA polymerase Beta; and ThermoPhi DNA polymerase.

In some embodiments, the amplification reaction components comprise one or more hyperthermophile DNA polymerases (e.g., hyperthermophile DNA polymerases that are thermostable at high temperatures). The hyperthermophile DNA polymerase can have a half-life of about 5 to 10 hours at 95° C. and a half-life of about 1 to 3 hours at 100° C. For example, the amplification reaction components can comprise one or more hyperthermophile DNA polymerases from Archaea (e.g., hyperthermophile DNA polymerases from Thermococcus, or hyperthermophile DNA polymerases from Thermococcaceaen archaean). In some embodiments, amplification reaction components comprise one or more hyperthermophile DNA polymerases from Pyrococcus, Methanococcaceae, Methanococcus, or Thermus. In some embodiments, amplification reaction components comprise one or more hyperthermophile DNA polymerases from Thermus thermophiles.

In some embodiments, amplification reaction components comprise a hyperthermophile DNA polymerase or functional fragment thereof. A functional fragment generally retains one or more functions of a full-length polymerase, for example, the capability to polymerize DNA (e.g., in an amplification reaction). In some instances, a functional fragment performs a function (e.g., polymerization of DNA in an amplification reaction) at a level that is at least about 50%, at least about 75%, at least about 90%, at least about 95% the level of function for a full length polymerase. Levels of polymerase activity can be assessed, for example, using a detectable nucleic acid amplification method, such as a detectable nucleic acid amplification method described herein. In some embodiments, amplification reaction components comprise a hyperthermophile DNA polymerase comprising an amino acid sequence of SEQ ID NO: 1 or SEQ ID NO: 2, or a functional fragment of SEQ ID NO: 1 or SEQ ID NO: 2.

In some embodiments, amplification reaction components (e.g., one or more amplification reagents) comprise a polymerase comprising an amino acid sequence that is at least about 90% identical to a hyperthermophile polymerase or a functional fragment thereof. In some embodiments, amplification reaction components comprise a polymerase comprising an amino acid sequence that is at least about 90%, 95%, or 99% identical to the amino acid sequence of SEQ ID NO: 1 or SEQ ID NO: 2, or a functional fragment thereof.

The polymerase can possess reverse transcription capabilities. In such embodiments, the amplification reaction can amplify RNA targets, for example, in a single step without the use of a separate reverse transcriptase. Non-limiting examples of polymerases that possess reverse transcriptase capabilities include Bst (large fragment), 9°N DNA polymerase, 9°Nm™ DNA polymerase, Therminator™, Therminator™ II, and the like). In some embodiments, amplification reaction components comprise one or more separate reverse transcriptases. In some embodiments, more than one polymerase is included in in an amplification reaction. For example, an amplification reaction may comprise a polymerase having reverse transcriptase activity and a second polymerase having no reverse transcriptase activity.

In some embodiments, one or more polymerases having exonuclease activity are used during amplification. In some embodiments, one or more polymerases having no or low exonuclease activity are used during amplification. In some embodiments, a polymerase having no or low exonuclease activity comprises one or more modifications (e.g., amino acid substitutions) that reduce or eliminate the exonuclease activity of the polymerase. For example, a modified polymerase having low exonuclease activity can have 10% or less exonuclease activity compared to an unmodified polymerase, for example less than about 10%, 9%, 8%, 7%, 6%, 5%, 4%, 3%, 2%, or 1% exonuclease activity compared to an unmodified polymerase. In some embodiments, a polymerase has no or low 5′ to 3′ exonuclease activity, and/or no or low 3′ to 5′ exonuclease activity. In some embodiments, a polymerase has no or low single strand dependent exonuclease activity, and/or no or low double strand dependent exonuclease activity. Non limiting examples of the modifications that can reduce or eliminate exonuclease activity for a polymerase include one or more amino acid substitutions at position 141 and/or 143 and/or 458 of SEQ ID NO: 1 (e.g., D141A, E143A, E143D and A485L), or at a position corresponding to position 141 and/or 143 and/or 458 of SEQ ID NO: 1.

### Detection and Quantification

The methods described herein can comprise detecting and/or quantifying nucleic acid amplification product(s). Amplification product(s) can be detected and/or quantified, for example, by any suitable detection and/or quantification method described herein. Non-limiting examples of detection and/or quantification methods include molecular beacon (e.g., real-time, endpoint), lateral flow, fluorescence resonance energy transfer (FRET), fluorescence polarization (FP), surface capture, 5′ to 3′ exonuclease hydrolysis probes (e.g., TAQMAN), intercalating/binding dyes, absorbance methods (e.g., colorimetric, turbidity), electrophoresis (e.g., gel electrophoresis, capillary electrophoresis), mass spectrometry, nucleic acid sequencing, digital amplification, a primer extension method (e.g., iPLEX™), Molecular Inversion Probe (MIP) technology from Affymetrix, restriction fragment length polymorphism (RFLP analysis), allele specific oligonucleotide (ASO) analysis, methylation-specific PCR (MSPCR), pyrosequencing analysis, acycloprime analysis, Reverse dot blot, GeneChip microarrays, Dynamic allele-specific hybridization (DASH), Peptide nucleic acid (PNA) and locked nucleic acids (LNA) probes, AlphaScreen, SNPstream, genetic bit analysis (GBA), Multiplex minisequencing, SNAPshot, GOOD assay, Microarray miniseq, arrayed primer extension (APEX), Microarray primer extension, Tag arrays, Coded microspheres, Template-directed incorporation (TDI), colorimetric oligonucleotide ligation assay (OLA), sequence-coded OLA, microarray ligation, ligase chain reaction, padlock probes, invader assay, hybridization using at least one probe, hybridization using at least one fluorescently labeled probe, cloning and sequencing, the use of hybridization probes and quantitative real time polymerase chain reaction (QRT-PCR), nanopore sequencing, chips and combinations thereof. In some embodiments, detecting a nucleic acid amplification product comprises use of a real-time detection method (i.e., product is detected and/or continuously monitored during an amplification process). In some embodiments, detecting a nucleic acid amplification product comprises use of an endpoint detection method (i.e., product is detected after completing or stopping an amplification process). Nucleic acid detection methods may also employ the use of labeled nucleotides incorporated directly into a target sequence or into probes containing complementary sequences to a target. Such labels may be radioactive and/or fluorescent in nature and can be resolved in any of the manners discussed herein. In some embodiments, quantification of a nucleic acid amplification product may be achieved using one or more detection methods described below. In some embodiments, the detection method can be used in conjunction with a measurement of signal intensity, and/or generation of (or reference to) a standard curve and/or look-up table for quantification of a nucleic acid amplification product.

Detecting a nucleic acid amplification product can comprise use of molecular beacon technology. The term molecular beacon generally refers to a detectable molecule, where the detectable property of the molecule is detectable under certain conditions, thereby enabling the molecule to function as a specific and informative signal. Non-limiting examples of detectable properties include optical properties (e.g., fluorescence), electrical properties, magnetic properties, chemical properties and time or speed through an opening of known size. Molecular beacons for detecting nucleic acid molecules can be, for example, hair-pin shaped oligonucleotides containing a fluorophore on one end and a quenching dye on the opposite end. The loop of the hair-pin can contain a probe sequence that is complementary to a target sequence and the stem is formed by annealing of complementary arm sequences located on either side of the probe sequence. A fluorophore and a quenching molecule can be covalently linked at opposite ends of each arm. Under conditions that prevent the oligonucleotides from hybridizing to its complementary target or when the molecular beacon is free in solution, the fluorescent and quenching molecules are proximal to one another preventing FRET. When the molecular beacon encounters a target molecule (e.g., a nucleic acid amplification product), hybridization can occur, and the loop structure is converted to a stable more rigid conformation causing separation of the fluorophore and quencher molecules leading to fluorescence. Due to the specificity of the probe, the generation of fluorescence generally is exclusively due to the synthesis of the intended amplified product. In some instances, a molecular beacon probe sequence hybridizes to a sequence in an amplification product that is identical to or complementary to a sequence in a target nucleic acid. In some instances, a molecular beacon probe sequence hybridizes to a sequence in an amplification product that is not identical to or complementary to a sequence in a target nucleic acid (e.g., hybridizes to a sequence added to an amplification product by way of a tailed amplification primer or ligation). Molecular beacons are highly specific and can discern a single nucleotide polymorphism. Molecular beacons also can be synthesized with different colored fluorophores and different target sequences, enabling simultaneous detection of several products in the same reaction (e.g., in a multiplex reaction). For quantitative amplification processes, molecular beacons can specifically bind to the amplified target following each cycle of amplification, and because non-hybridized molecular beacons are dark, it is not necessary to isolate the probe-target hybrids to quantitatively determine the amount of amplified product. The resulting signal is proportional to the amount of amplified product. Detection using molecular beacons can be done in real time or as an end-point detection method.

Detecting a nucleic acid amplification product can comprise use of lateral flow. Use of lateral flow typically includes use of a lateral flow device including but not limited to dipstick assays and thin layer chromatographic plates with various appropriate coatings. Immobilized on the flow path are various binding reagents for the sample, binding partners or conjugates involving binding partners for the sample and signal producing systems. Detection can be achieved by, for example, enzymatic detection, nanoparticle detection, colorimetric detection, and fluorescence detection. Nucleic acids can be captured on lateral flow devices, for example by antibody-dependent and/or antibody-independent methods. Antibody-dependent capture comprises an antibody capture line and a labeled probe of complementary sequence to the target. Antibody-independent capture uses non-covalent interactions between two binding partners, for example, the high affinity and irreversible linkage between a biotinylated probe and a streptavidin line. Capture probes can be immobilized directly on lateral flow membranes. Both antibody-dependent and antibody-independent methods may be used, for example, for detecting amplification products generated in a multiplex reaction.

Detecting a nucleic acid amplification product can comprise use of FRET which is an energy transfer mechanism between two chromophores: a donor and an acceptor molecule. Briefly, a donor fluorophore molecule is excited at a specific excitation wavelength. The subsequent emission from the donor molecule as it returns to its ground state may transfer excitation energy to the acceptor molecule through a long range dipole-dipole interaction. The emission intensity of the acceptor molecule can be monitored and is a function of the distance between the donor and the acceptor, the overlap of the donor emission spectrum and the acceptor absorption spectrum and the orientation of the donor emission dipole moment and the acceptor absorption dipole moment. FRET can be useful for quantifying molecular dynamics, for example, in DNA-DNA interactions as described for molecular beacons. For monitoring the production of a specific product, a probe can be labeled with a donor molecule on one end and an acceptor molecule on the other. Probe-target hybridization brings a change in the distance or orientation of the donor and acceptor and FRET change is observed.

Detecting a nucleic acid amplification product can comprise use of fluorescence polarization (FP). FP techniques are based on the principle that a fluorescently labeled compound when excited by linearly polarized light will emit fluorescence having a degree of polarization inversely related to its rate of rotation. Therefore, when a molecule such as a tracer-nucleic acid conjugate, for example, having a fluorescent label is excited with linearly polarized light, the emitted light remains highly polarized because the fluorophore is constrained from rotating between the time light is absorbed and emitted. When a free tracer compound (i.e., unbound to a nucleic acid) is excited by linearly polarized light, its rotation is much faster than the corresponding tracer-nucleic acid conjugate and the molecules are more randomly oriented, therefore, the emitted light is depolarized. Thus, fluorescence polarization provides a quantitative means for measuring the amount of tracer-nucleic acid conjugate produced in an amplification reaction.

Detecting a nucleic acid amplification product can comprise use of surface capture, accomplished for example by the immobilization of specific oligonucleotides to a surface producing a biosensor that is both highly sensitive and selective. Example surfaces that can be used include gold and carbon, and a surface capture method may use a number of covalent or noncovalent coupling methods to attach a probe to the surface. Detecting a nucleic acid amplification product can comprise use of 5′ to 3′ exonuclease hydrolysis probes (e.g., TAQMAN). TAQMAN probes, for example, are hydrolysis probes that can increase the specificity of a quantitative amplification method (e.g., quantitative PCR). The TAQMAN probe principle relies on 1) the 5′ to 3′ exonuclease activity of Taq polymerase to cleave a dual-labeled probe during hybridization to a complementary target sequence and 2) fluorophore-based detection. A resulting fluorescence signal permits quantitative measurements of the accumulation of amplification product during the exponential stages of amplification, and the TAQMAN probe can significantly increase the specificity of the detection.

Detecting a nucleic acid amplification product can comprise use of intercalating and/or binding dyes, for example dyes that specifically stain nucleic acid (e.g., intercalating dyes exhibit enhanced fluorescence upon binding to DNA or RNA). Dyes can include DNA or RNA intercalating fluorophores, including but not limited to, SYTOR 82, acridine orange, ethidium bromide, Hoechst dyes, PicoGreen®, propidium iodide, SYBR® I (an asymmetrical cyanine dye), SYBR® II, TOTO (a thiaxole orange dimer) and YOYO (an oxazole yellow dimer). Detecting a nucleic acid amplification product can comprise use of absorbance methods (e.g., colorimetric, turbidity). In some embodiments, detection and/or quantitation of nucleic acid can be achieved by directly converting absorbance (e.g., UV absorbance measurements at 260 nm) to concentration. Direct measurement of nucleic acid can be converted to concentration using the Beer Lambert law which relates absorbance to concentration using the path length of the measurement and an extinction coefficient. Detecting a nucleic acid amplification product can comprise use of electrophoresis (e.g., gel electrophoresis, capillary electrophoresis) and/or use of mass spectrometry. Mass Spectrometry is an analytical technique that can be used to determine the structure and quantity of a nucleic acid and can be used to provide rapid analysis of complex mixtures. Following amplification, samples can be ionized, the resulting ions separated in electric and/or magnetic fields according to their mass-to-charge ratio, and a detector measures the mass-to-charge ratio of ions. Mass spectrometry methods include, for example, MALDI, MALDI-TOF, and electrospray. These methods may be combined with gas chromatography (GC/MS) and liquid chromatography (LC/MS). Mass spectrometry (e.g., matrix-assisted laser desorption/ionization mass spectrometry (MALDI MS)) can be high throughput due to high-speed signal acquisition and automated analysis off solid surfaces.

Detecting a nucleic acid amplification product can comprise use of nucleic acid sequencing. The entire sequence or a partial sequence of an amplification product can be determined, and the determined nucleotide sequence may be referred to as a read. For example, linear amplification products may be analyzed directly without further amplification (e.g., by using single-molecule sequencing methodology). In some embodiments, linear amplification products is subject to further amplification and then analyzed (e.g., using sequencing by ligation or pyrosequencing methodology). Non-limiting examples of sequencing methods include single-end sequencing, paired-end sequencing, reversible terminator-based sequencing, sequencing by ligation, pyrosequencing, sequencing by synthesis, single-molecule sequencing, multiplex sequencing, solid phase single nucleotide sequencing, and nanopore sequencing. Detecting a nucleic acid amplification product can comprise use of digital amplification (e.g., digital PCR). Systems for digital amplification and analysis of nucleic acids are available (e.g., Fluidigm® Corporation).

### Acidic Compositions

The acidic compositions (e.g., lysis buffers, elution buffers, resuspension buffers) for dissociation of dsDNA disclosed herein can be made with low concentration of an acidic agent, such as 10-20 mM HCl. Other inorganic acids or organic acids known in the art can also be used. The amplification reaction buffer used for neutralization of pH can be 30-50 mM Tris pH 8.8 buffer. In some embodiments, the acidic composition can contain an acidic buffer such as glycine/-HCl to increase the acidic buffer capacity and compensate for pH variability found in clinical samples (e.g., urine or nasal swabs). Without being bound by any particular theory, in some embodiments, at low pH, the high concentration of positively charged hydrogen ions disrupts the secondary structure of nucleic acids by breaking down non-covalent hydrogen bonding and hydrophobic interactions, destabilizing the double helix structure and causing the strands to separate.

Disclosed herein include acidic compositions for lysing biological entities and denaturing dsDNA comprised therein. In some embodiments, the acidic composition comprises: a monovalent salt and/or a divalent salt; one or more surfactants; and an acidic agent, wherein the acidic agent is present in the acidic composition at a concentration of less than 100 mM, and wherein the acidic composition has a pH less than 4. The monovalent salt can be present in the acidic composition at a concentration of less than 30 mM. The divalent salt can be present in the acidic composition at a concentration of less than 15 mM.

In some embodiments, the acidic composition has a pH in the range of about 1 to about 3.9 (e.g., about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or a number or a range between any two of these values.). In some embodiments, the acidic composition has a pH of about 2. In some embodiments, the acidic composition has a pH of about 2, about 2.1, about 2.2, about 2.3, about 2.4, and about 2.5. The pH as used herein can be determined by any known method of calculating or measuring the pH of an aqueous solution. In some embodiments, the acidic composition does not comprise glycerol, formamide, or urea.

In some embodiments, the percentages of the acidic composition components disclosed herein are provided as % w/w, % m/v, % v/v, % m/w, % w/v, or variations thereof. In some embodiments, the percentage (% w/w, % m/v, % v/v, % m/w, % w/v, or variations thereof) of the acidic composition components disclosed herein (e.g., one or more surfactants) within the acidic composition can be, or be about, 0.000000001%, 0.00000001%, 0.0000001%, 0.000001%, 0.00001%, 0.0001%, 0.001%, 0.01%, 0.02%, 0.03%, 0.04%, 0.05%, 0.06%, 0.07%, 0.08%, 0.09%, 0.10%, 0.11%, 0.12%, 0.13%, 0.14%, 0.15%, 0.16%, 0.17%, 0.18%, 0.19%, 0.20%, 0.21%, 0.22%, 0.23%, 0.24%, 0.25%, 0.26%, 0.27%, 0.28%, 0.29%, 0.30%, 0.31%, 0.32%, 0.33%, 0.34%, 0.35%, 0.36%, 0.37%, 0.38%, 0.39%, 0.40%, 0.41%, 0.42%, 0.43%, 0.44%, 0.45%, 0.46%, 0.47%, 0.48%, 0.49%, 0.50%, 0.51%, 0.52%, 0.53%, 0.54%, 0.55%, 0.56%, 0.57%, 0.58%, 0.59%, 0.60%, 0.61%, 0.62%, 0.63%, 0.64%, 0.65%, 0.66%, 0.67%, 0.68%, 0.69%, 0.70%, 0.71%, 0.72%, 0.73%, 0.74%, 0.75%, 0.76%, 0.77%, 0.78%, 0.79%, 0.80%, 0.81%, 0.82%, 0.83%, 0.84%, 0.85%, 0.86%, 0.87%, 0.88%, 0.89%, 0.90%, 0.91%, 0.92%, 0.93%, 0.94%, 0.95%, 0.96%, 0.97%, 0.98%, 0.99%, 1.0%, 1.01%, 1.02%, 1.03%, 1.04%, 1.05%, 1.06%, 1.07%, 1.08%, 1.09%, 1.10%, 1.11%, 1.12%, 1.13%, 1.14%, 1.15%, 1.16%, 1.17%, 1.18%, 1.19%, 1.20%, 1.21%, 1.22%, 1.23%, 1.24%, 1.25%, 1.26%, 1.27%, 1.28%, 1.29%, 1.30%, 1.31%, 1.32%, 1.33%, 1.34%, 1.35%, 1.36%, 1.37%, 1.38%, 1.39%, 1.40%, 1.41%, 1.42%, 1.43%, 1.44%, 1.45%, 1.46%, 1.47%, 1.48%, 1.49%, 1.50%, 1.51%, 1.52%, 1.53%, 1.54%, 1.55%, 1.56%, 1.57%, 1.58%, 1.59%, 1.60%, 1.61%, 1.62%, 1.63%, 1.64%, 1.65%, 1.66%, 1.67%, 1.68%, 1.69%, 1.70%, 1.71%, 1.72%, 1.73%, 1.74%, 1.75%, 1.76%, 1.77%, 1.78%, 1.79%, 1.80%, 1.81%, 1.82%, 1.83%, 1.84%, 1.85%, 1.86%, 1.87%, 1.88%, 1.89%, 1.90%, 1.91%, 1.92%, 1.93%, 1.94%, 1.95%, 1.96%, 1.97%, 1.98%, 1.99%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, or a number or a range between any two of these values. In some embodiments, the percentages of acidic composition components disclosed herein are described with regards to their final concentration once the acidic composition is contacted with a sample comprising biological entities. Additionally, while in some embodiments, the acidic composition components disclosed herein are described in with regards to working concentrations (e.g., 1×) of the acidic composition, the disclosure also contemplates concentrated versions of the disclosed acidic compositions (e.g., a 2× acidic composition).

### Acidic Agents

The compositions (e.g., acidic compositions) disclosed herein can comprise one or more acidic agents. The acidic agent can comprise an organic acid, an inorganic acid, or both. The acidic agent can be hydrochloric acid, glycine hydrochloride, acetic acid, citric acid, phosphoric acid, or a combination thereof.

The acidic agent can vary depending on the embodiment. The inorganic acid can be hydrochloric acid (HCl). The inorganic acid can comprise one or more of hydrofluoric acid, hydrochloric acid, hydrobromic acid, hydroiodic acid, nitric acid, nitrous acid, phosphoric acid, phosphinic acid, phosphonic acid, sulfonic acid, sulfuric acid, sulfurous acid, disulfuric acid carbonic acid and boric acid. The organic acid can comprise one or more of acetic acid, C₂H₅COOH, C₃H₇COOH, C₄H₉COOH, (COOH)₂, CH₂ (COOH)₂, C₂H₄ (COOH)₂, C₃H₆ (COOH)₂, C₄H₈ (COOH)₂, C₅H₁₀ (COOH)₂, fumaric acid, maleic acid, malonic acid, lactic acid, citric acid, tartaric acid, oxalic acid, ascorbic acid, benzoic acid, salicylic acid, phthalic acid, pyruvic acid, L-aspartic acid, D-aspartic acid, carbonic acid, formic acid, methanesulfonic acid, ethanesulfonic acid, benzenesulfonic acid, glucosamine sulphate, L-threonic acid, camphoric acid, gluconic acid, L-glutamic acid, D-glutamic acid, trifluoroacetic acid or ranelic acid.

In some embodiments, the acidic agent is an acidic buffer (e.g., an acidic buffer solution). An acidic buffer solution can be one which has a pH less than 7. Acidic buffer solutions can be made from a weak acid and one of its salts (e.g., a sodium salt), or can be obtained from a commercial source. An example is a mixture of ethanoic acid and sodium ethanoate in solution. In this case, if the solution contained equal molar concentrations of both the acid and the salt, it would have a pH of 4.76. Thus, as used herein, the term “acidic buffer” shall be given its ordinary meaning, and shall also refer to a compound or compounds that, when added to an aqueous solution, reduces the pH and causes the resulting solution to resist a change in pH when the solution is mixed with solutions of lower or higher pH. The acidic buffer can have a pKa below about 7. The acidic buffer can have a pKa below about 7, below about 6, below about 5, below about 4 and below about 3. Acidic buffers with all individual values and ranges of pKa below about 7 are included in the present disclosure. Examples of acidic buffers suitable for acidic compositions described herein include, but are not limited to, phosphate, citrate, iso-citrate, acetate, succinate, ascorbic, formic, lactic, sulfuric, hydrochloric, nitric, benzoic, boric, butyric, capric, caprilic, carbonic, carboxylic, oxalic, pyruvic, phthalic, adipic, citramalic, fumaric, glycolic, tartaric, isotartaric, lauric, maleic, isomalic, malonic, orotic, propionic, methylpropionic, polyacrylic, succinic, salicylic, 5-sulfosalicylic, valeric, isovaleric, uric, and combinations thereof, such as a combination of hydrochloric acid and glycine that has a pH of about 2.2, as well as other suitable acids and bases, as known in the art. Other suitable acidic buffers are mixtures of an acid and one or more salts or buffering agents. For example, an acidic buffer suitable for use herein can be prepared using glycine in combination with hydrochloric acid in appropriate concentrations. The acidic buffer can comprise glycine-HCl. The acidic buffer can comprise 10.0 mM glycine, 8.8 mM HCl, or both. The pH of the acidic buffer can be about 1.0 to about 6.0 (e.g., 2.2).

The concentration of the acidic agent that is present in the acidic composition can vary. The acidic agent can be present in the acidic composition at a concentration of less than about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 30 mM, 31 mM, 32 mM, 33 mM, 34 mM, 35 mM, 36 mM, 37 mM, 38 mM, 39 mM, 40 mM, 41 mM, 42 mM, 43 mM, 44 mM, 45 mM, 46 mM, 47 mM, 48 mM, 49 mM, 50 mM, 51 mM, 52 mM, 53 mM, 54 mM, 55 mM, 56 mM, 57 mM, 58 mM, 59 mM, 60 mM, 61 mM, 62 mM, 63 mM, 64 mM, 65 mM, 66 mM, 67 mM, 68 mM, 69 mM, 70 mM, 71 mM, 72 mM, 73 mM, 74 mM, 75 mM, 76 mM, 77 mM, 78 mM, 79 mM, 80 mM, 81 mM, 82 mM, 83 mM, 84 mM, 85 mM, 86 mM, 87 mM, 88 mM, 89 mM, 90 mM, 91 mM, 92 mM, 93 mM, 94 mM, 95 mM, 96 mM, 97 mM, 98 mM, 99 mM, 100 mM, or a number or a range between any two of these values. The acidic agent can be present in the acidic composition at a concentration in the range of about 1 mM to about 100 mM, e.g., about 10 mM or about 8.8 mM.

### Salts

The acidic composition (e.g., lysis buffer) can comprise a monovalent salt and/or a divalent salt (e.g., magnesium), which, without being bound by any particular theory, can suppress depurination in some embodiments. In some embodiments, the acidic composition comprises low concentrations of monovalent salt and/or divalent salt. Without being bound by any particular theory, this may be due to neutralizing negative charges on phosphate groups on the DNA backbones.

The monovalent salt and/or the divalent salt of the acidic compositions (e.g., lysis buffers) provided herein can comprise a sodium salt, a potassium salt, a calcium salt, a magnesium salt, or any combination thereof. The monovalent salt can be selected from the group consisting of ammonium sulfate, ammonium chloride, sodium fluoride, sodium chloride, sodium bromide, sodium iodide, potassium fluoride, potassium chloride, potassium bromide, and potassium iodide. The divalent salt can be selected from the group consisting of magnesium sulfate, calcium chloride, magnesium chloride, copper (II) chloride, zinc chloride, calcium oxide, magnesium oxide, barium oxide, sodium sulfate, calcium sulfate, copper (II) sulfate, potassium carbonate, and sodium carbonate. In some embodiments, the monovalent salt is ammonium sulfate. In some embodiments, the divalent salt is magnesium sulfate.

In some embodiments, the monovalent salt and/or the divalent salt is present in the acidic composition at a concentration of less than 15 mM, for example about 0.1 mM, 0.2 mM, 0.3 mM, 0.4 mM, 0.5 mM, 0.6 mM, 0.7 mM, 0.8 mM, 0.9 mM, 1 mM, 2 mM, 3 mM, 4 mM, 5 mM, 6 mM, 7 mM, 8 mM, 9 mM, 10 mM, 11 mM, 12 mM, 13 mM, 14 mM, 15 mM, 16 mM, 17 mM, 18 mM, 19 mM, 20 mM, 21 mM, 22 mM, 23 mM, 24 mM, 25 mM, 26 mM, 27 mM, 28 mM, 29 mM, 29.9 mM, or a number or a range between any two of these values. The monovalent salt and/or the divalent salt can be present in the acidic composition at a concentration in the range of about 1 mM to about 14 mM, for example a concentration of about 5 mM or about 4 mM.

### Surfactants

The acid compositions (e.g., lysis buffers) provided herein can comprise one or more lytic agents (e.g., surfactants, detergents) such as, for example, a cationic surfactant, an anionic surfactant, a non-ionic surfactant, and an amphoteric surfactant. The anionic surfactant can comprise NH₄⁺, K⁺, Na⁺, or Li⁺ as a counter ion. The cationic surfactant can comprise I⁻, Br⁻, or Cl⁻ as a counter ion.

The anionic surfactant can be selected from the group consisting of potassium laurate, triethanolamine stearate, ammonium lauryl sulfate, lithium dodecyl sulfate, sodium lauryl sulfate, sodium alkyl sulfate (C8-16), SDS, alkyl polyoxyethylene sulfate, sodium alginate, dioctyl sodium sulfosuccinate, phosphatidyl glycerol, phosphatidyl inositol, phosphatidylserine, phosphatidic acid and salts thereof, glyceryl ester, sodium carboxymethylcellulose, bile acid and acid, alkyl sulfonate, aryl sulfonate, alkyl phosphate, alkyl sulfonate, stearic acid and salts thereof, calcium stearate, phosphate, sodium carboxymethyl cellulose, dioctyl sulfosuccinate, dialkyl ester of sodium sulfosuccinic acid, phospholipid and calcium carboxymethyl cellulose.

The cationic surfactant can be, for example, quaternary ammonium compounds, benzalkonium chloride, cetyl trimethyl ammonium bromide, chitonic acid, lauryl dimethyl benzyl ammonium chloride, acyl carnitine hydrochloride, alkyl pyridinium halide, cetylpyridinium chloride, cationic lipids, polymethylmethacrylate trimethyl ammonium bromide, sulfonium compounds, polyvinylpyrrolidone-2-dimethylaminoethyl methacrylate dimethyl sulfate, hexadecyl trimethyl ammonium bromide, phosphonium compounds, quaternary ammonium compounds, benzyl-di(2-chloroethyl) ethyl ammonium bromide, coconut trimethyl ammonium chloride, coconut trimethyl ammonium bromide, coconut methyl dihydroxyethyl ammonium chloride, coconut methyl dihydroxyethyl ammonium bromide, decyl triethyl ammonium chloride, decyl dimethyl hydroxyethyl ammonium chloride, decyl dimethyl hydroxyethyl ammonium chloride bromide, C12-15-dimethyl hydroxyethyl ammonium chloride, C12-15-dimethyl hydroxyethyl ammonium chloride bromide, coconut dimethyl hydroxyethyl ammonium chloride, coconut dimethyl hydroxyethyl ammonium bromide, myristyl trimethyl ammonium methyl sulphate, lauryl dimethyl benzyl ammonium chloride, lauryl dimethyl benzyl ammonium bromide, lauryl dimethyl (ethenoxy) 4 ammonium chloride, lauryl dimethyl (ethenoxy) 4 ammonium bromide, N-alkyl (C12-18) dimethylbenzyl ammonium chloride, N-alkyl (C14-18) dimethyl-benzyl ammonium chloride, N-tetradecyldimethylbenzyl ammonium chloride monohydrate, dimethyl didecyl ammonium chloride, N-alkyl (C12-14) dimethyl 1-naphthylmethyl ammonium chloride, trimethylammonium halide alkyl-trimethylammonium salts, dialkyl-dimethylammonium salts, lauryl trimethyl ammonium chloride, ethoxylated alkyamidoalkyldialkylammonium salts, ethoxylated trialkyl ammonium salts, dialkylbenzene dialkylammonium chloride, N-didecyldimethyl ammonium chloride, N-tetradecyldimethylbenzyl ammonium chloride monohydrate, N-alkyl (C12-14) dimethyl 1-naphthylmethyl ammonium chloride, dodecyldimethylbenzyl ammonium chloride, dialkyl benzenealkyl ammonium chloride, lauryl trimethyl ammonium chloride, alkylbenzyl methyl ammonium chloride, alkyl benzyl dimethyl ammonium bromide, C8-16 trimethyl ammonium bromide, C8-16 trimethyl ammonium chloride, C15 trimethyl ammonium bromide, C17 trimethyl ammonium bromide, dodecylbenzyl triethyl ammonium chloride, polydiallyldimethylammonium chloride, dimethyl ammonium chloride, alkyldimethylammonium halogenide, tricetyl methyl ammonium chloride, decyltrimethylammonium bromide, dodecyltriethylammonium bromide, tetradecyltrimethylammonium bromide, methyl trioctylammonium chloride, POLYQUAT 10, tetrabutylammonium bromide, benzyl trimethylammonium bromide, choline ester, benzalkonium chloride, stearalkonium chloride, cetyl pyridinium bromide, cetyl pyridinium chloride, halide salts of quaternized polyoxyethylalkylamines, MIRAPOL Alkaquat, alkyl pyridinium salts, amine, amine salts, imide azolinium salts, protonated quaternary acrylamides, methylated quaternary polymers, cationic gua gum, benzalkonium chloride, dodecyl trimethyl ammonium bromide, triethanolamine, or poloxamine.

The non-ionic surfactant can be, for example, polyoxyethylene fatty alcohol ether, polyoxyethylene sorbitan fatty acid ester, polyoxyethylene fatty acid ester, polyoxyethylene alkyl ether, polyoxyethylene castor oil derivatives, sorbitan ester, glyceryl ester, glycerol monostearate, polyethylene glycol, polypropylene glycol, polypropylene glycol ester, cetyl alcohol, cetostearyl alcohol, stearyl alcohol, aryl alkyl polyether alcohol, polyoxyethylene polyoxypropylene copolymers, poloxamer, poloxamine, methylcellulose, hydroxycellulose, hydroxymethylcellulose, hydroxyethylcellulose, hydroxypropylcellulose, hydroxypropylmethylcellulose, hydroxypropylmethylcellulose phthalate, noncrystalline cellulose, polysaccharides, starch, starch derivatives, hydroxyethyl starch, polyvinyl alcohol, triethanolamine stearate, amine oxide, dextran, glycerol, gum acacia, cholesterol, tragacanth, polyvinylpyrrolidone, alkyl sulfates, alkyl sulfonates, fatty acid soaps, salts of hydrox-, hydroperoxy-, polyhydroxy-, epoxy-fatty acids, salts of mono- and polycarboxylic acids, prostanoic acid and prostaglandins, leukotrienes and lipoxines, alkyl phosphates, alkyl phosphonates, sodium-dialkyl sufosuccinate, n-alkyl ethoxylated sulfates, cholate and desoxycholate of bile salts, perfluorocarboxylic acids, fluoroacliphatic phosphonates, or fluoroaliphatic sulphates.

The lytic agents provided herein can be capable of acting as a denaturing agent. “Denaturing agent” or “denaturant,” as used herein, shall be given its ordinary meaning and include any compound or material which will cause a reversible unfolding of a protein. The strength of a denaturing agent or denaturant will be determined both by the properties and the concentration of the particular denaturing agent or denaturant. Suitable denaturing agents or denaturants can be chaotropes, detergents, organic solvents, water miscible solvents, phospholipids, or a combination of two or more such agents. Suitable chaotropes include, but are not limited to, urea, guanidine, and sodium thiocyanate. Useful detergents can include, but are not limited to, strong detergents such SDS, or polyoxyethylene ethers (e.g., Tween or Triton detergents), Sarkosyl, mild non-ionic detergents (e.g., digitonin), mild cationic detergents such as N->2,3-(Dioleyoxy)-propyl-N,N,N-trimethylammonium, mild ionic detergents (e.g., sodium cholate or sodium deoxycholate) or zwitterionic detergents including, but not limited to, sulfobetaines (Zwittergent), 3-(3-chlolamidopropyl) dimethylammonio-1-propane sulfate (CHAPS), and 3-(3-chlolamidopropyl) dimethylammonio-2-hydroxy-1-propane sulfonate (CHAPSO). Organic, water miscible solvents such as acetonitrile, lower alkanols (especially C2-C4 alkanols such as ethanol or isopropanol), or lower alkandiols (especially C2-C4 alkandiols such as ethylene-glycol) can be used as denaturants. Phospholipids can be naturally occurring phospholipids such as phosphatidylethanolamine, phosphatidylcholine, phosphatidylserine, and phosphatidylinositol or synthetic phospholipid derivatives or variants such as dihexanoylphosphatidylcholine or diheptanoylphosphatidylcholine.

The one or more surfactants can comprise one or more of a cationic surfactant, an anionic surfactant, a non-ionic surfactant, and an amphoteric surfactant. In some embodiments, the acidic composition does not comprise both an anionic surfactant and a cationic surfactant. The one or more surfactants can be present in the acidic composition at a concentration in the range of about 0.01% to about 2% weight by volume (% w/v) of the acidic composition. The one or more surfactants can be present in the acidic composition at a concentration of about 0.1% weight by volume (% w/v) of the acidic composition. In some embodiments, the acidic composition further comprises a tween surfactant, for example a tween surfactant selected from Tween 20, Tween 40, Tween 45, Tween 60, Tween 65, Tween 80, Tween 81 and Tween 85. The tween surfactant can comprise about 0.01% (w/v) of the acidic composition.

Useful anionic surfactants herein include the water-soluble salts of alkyl sulphates and alkyl ether sulphates having from 10 to 18 carbon atoms in the alkyl radical and the water-soluble salts of sulphonated monoglycerides of fatty acids having from 10 to 18 carbon atoms. Sodium lauryl sulphate and sodium coconut monoglyceride sulphonates are examples of anionic surfactants of this type.

Suitable cationic surfactants can be broadly defined as derivatives of aliphatic quaternary ammonium compounds having one long alkyl chain containing from about 8 to 18 carbon atoms such as lauryl trimethylammonium chloride; cetyl pyridinium chloride; benzalkonium chloride; CTAB; di-isobutylphenoxyethyl-dimethylbenzylammonium chloride; coconut alkyltrimethyl-ammonium nitrite; cetyl pyridinium fluoride; etc. Certain cationic surfactants can also act as germicides in the compositions disclosed herein.

Suitable nonionic surfactants that can be used in the compositions, methods and kits of the present disclosure can be broadly defined as compounds produced by the condensation of alkylene oxide groups (hydrophilic in nature) with an organic hydrophobic compound which can be aliphatic and/or aromatic in nature. Examples of suitable nonionic surfactants include the poloxamers; sorbitan derivatives, such as sorbitan di-isostearate; ethylene oxide condensates of hydrogenated castor oil, such as PEG-30 hydrogenated castor oil; ethylene oxide condensates of aliphatic alcohols or alkyl phenols; products derived from the condensation of ethylene oxide with the reaction product of propylene oxide and ethylene diamine; long chain tertiary amine oxides; long chain tertiary phosphine oxides; long chain dialkyl sulphoxides and mixtures of such materials. These materials are useful for stabilizing foams without contributing to excess viscosity build for the consumer product composition.

Zwitterionic surfactants can be broadly described as derivatives of aliphatic quaternary ammonium, phosphonium, and sulphonium compounds, in which the aliphatic radicals can be straight chain or branched, and wherein one of the aliphatic substituents contains from about 8 to 18 carbon atoms and one contains an anionic water-solubilizing group, e.g., carboxy, sulphonate, sulphate, phosphate or phosphonate.

Exemplary anionic, single-chain surface active agents include alkyl sulfates, alkyl sulfonates, alkyl benzene sulfonates, and saturated or unsaturated fatty acids and their salts. Moieties comprising the polar head group in the cationic surfactant can include, for example, quaternary ammonium, pyridinium, sulfonium, and/or phosphonium groups. For example, the polar head group can include trimethylammonium. Exemplary cationic, single-chain surface active agents include alkyl trimethylammonium halides, alkyl trimethylammonium tosylates, and N-alkyl pyridinium halides.

Alkyl sulfates can include sodium octyl sulfate, SDeS, SDS, and sodium tetra-decyl sulfate. Alkyl sulfonates can include sodium octyl sulfonate, sodium decyl sulfonate, and sodium dodecyl sulfonate. Alkyl benzene sulfonates can include sodium octyl benzene sulfonate, sodium decyl benzene sulfonate, and sodium dodecyl benzene sulfonate. Fatty acid salts can include sodium octanoate, sodium decanoate, sodium dodecanoate, and the sodium salt of oleic acid.

Alkyl trimethylammonium halides can include octyl trimethylammonium bromide, decyl trimethylammonium bromide, dodecyl trimethylammonium bromide, myristyl trimethylammonium bromide, and CTAB. Alkyl trimethylammonium tosylates can include octyl trimethylammonium tosylate, decyl trimethylammonium tosylate, dodecyl trimethylammonium tosylate, myristyl trimethylammonium tosylate, and cetyl trimethylammonium tosylate. For example, N-alkyl pyridinium halides can include decyl pyridinium chloride, dodecyl pyridinium chloride, cetyl pyridinium chloride, decyl pyridinium bromide, dodecyl pyridinium bromide, cetyl pyridinium bromide, decyl pyridinium iodide, dodecyl pyridinium iodide, cetyl pyridinium iodide.

The cationic surfactant can comprise dodecyltrimethylammonium bromide, tetradecyltrimethylammonium bromide, cetyltrimethylammonium bromide, cetyldimethylethylammonium bromide, (C1 to C30 alkyl)-trimethylammonium bromide, a (C1 to C30 alkyl) amine, a (C1 to C30 alkyl) imidazoline, ethoxylated amine, a quaternary compound, a quaternary ester, a (C1 to C30 alkyl) amine oxide, lauramine oxide, dicetyldimonium chloride, cetrimonium chloride, a primary polyethoxylated fatty amine salt, a secondary polyethoxylated fatty amine salt, a tertiary polyethoxylated fatty amine salt, a quaternary ammonium salt, a tetra (C1 to C30 alkyl) ammonium halide, a (C1 to C30 alkyl) amide-(C1 to C30-alkyl) ammonium halide, a tri (C1 to C30 alkyl) benzylammonium halide, a tri (C1 to C30 alkyl) hydroxy-(C1 to C30 alkyl) ammonium halide, a (C1 to C30 alkyl) pyridinium chloride, a (C1 to C30 alkyl) pyridinium bromide, a amine oxide, or a combination thereof.

The anionic surfactant can comprise SDS, a (C6 to C30 alkyl) benzene sulfonate, a C6 to C30 alpha olefin sulfonate, a paraffin sulfonate, a (C6 to C30 alkyl) ester sulfonate, a (C6 to C30 alkyl) sulfate, a (C6 to C30 alkyl alkoxy) sulfate, a (C6 to C30 alkyl) sulfonate, a (C6 to C30 alkyl alkoxy) carboxylate, a (C6 to C30 alkyl alkoxylated) sulfate, a mono (C1 to C30 alkyl) (ether) phosphate, a di (C6 to C30 alkyl) (ether) phosphate, a (C6 to C30 alkyl) sarcosinate, a sulfosuccinate, sodium bis(2-ethylhexyl) sulfosuccinate, ethoxylate 4-nonylphenyl ether glycolic acid, a (C1 to C30 alkyl) isethionate, taurate, ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium lauryl sulfate, potassium laureth sulfate, sodium lauryl phosphate, sodium tridecyl phosphate, sodium behenyl phosphate, sodium laureth-2 phosphate, sodium ceteth-3 phosphate, sodium trideceth-4 phosphate, sodium dilauryl phosphate, sodium ditridecyl phosphate, sodium ditrideceth-6 phosphate, sodium lauroyl sarcosinate, lauroyl sarcosine, cocoyl sarcosine, ammonium cocoyl sulfate, sodium cocoyl sulfate, sodium trideceth sulfate, sodium tridecyl sulfate, ammonium trideceth sulfate, ammonium tridecyl sulfate, sodium cocoyl isethionate, disodium laureth sulfosuccinate, sodium methyl oleoyl taurate, sodium laureth carboxylate, sodium trideceth carboxylate, sodium lauryl sulfate, potassium cocoyl sulfate, potassium lauryl sulfate, monoethanolamine cocoyl sulfate, sodium tridecyl benzene sulfonate, sodium dodecyl benzene sulfonate, or a combination thereof.

The non-ionic surfactant can comprise, for example, a C6 to C18 alkyl alcohol, a (C6 to C18 alkyl) phenol, a (C6 to C18 alkyl) ethoxylate, a (C6 to C18 alkyl) phenol (C₁ to C₃ alkoxylate), a block oxy (C₁ to C₃ alkylene) condensate of a C6 to C18 alkyl phenol, an oxy (C₁ to C₃ alkylene) condensate of alkanol, an oxyethylene/oxypropylene block copolymer, an amine oxide, a phosphine oxide, an alkylamine oxide having 8 to 50 carbon atoms, a mono or di (C8 to C30) alkyl alkanolamide, a (C6 to C30 alkyl) polysaccharide, a sorbitan fatty acid ester, a polyoxyethylene sorbitan fatty acid ester, a polyoxyethylene sorbitol ester, a polyoxyethylene nonylphenyl ether, a polyoxyethylenic acid, a polyoxyethylene alcohol, a coco monoethanolamide, a coco diethanolamide, a coco diglycoside, a (C8 to C30 alkyl) polyglycoside, cocamidopropyl, lauramine oxide, polyoxyethylene (20) sorbitan monolaurate, an ethoxylated linear C8 to C30 alcohol, cetearyl alcohol, lanolin alcohol, stearic acid, glyceryl stearate, polyethylene glycol 100 stearate, 4-(1,1,3,3-tetramethylbutyl) phenyl polyethylene glycol, polyoxyethylene (10) cetyl ether, eicosaethylene glycol octadecyl ether, and HO(CH₂CH₂O)₂₀(CH₂CH(CH₃)O)₇₀ (CH₂CH₂O)₂₀H.

The one or more surfactants can comprise about 0.001% (w/v) to about 2.0% (w/v) of the acidic composition (e.g., lysis buffer). The one or more surfactants can be capable of lysing biological entities to release sample nucleic acids comprised therein. The one or more surfactants can comprise about 0.001%, 0.01%, 0.1%, 1%, 2%, 3%, 4%, 5%, 6%, 7%, 8%, 9%, 10%, or a number or a range between any two of these values, (w/v) of the acidic composition. The one or more surfactants disclosed herein can comprise one or more of a cationic surfactant, an anionic surfactant, a non-ionic surfactant, and an amphoteric surfactant. The acidic composition further can comprise a tween surfactant. The tween surfactant can be selected from Tween 20, Tween 40, Tween 45, Tween 60, Tween 65, Tween 80, Tween 81 and Tween 85. The tween surfactant can comprise about 0.01% (w/v) to about 1.0% (w/v) of the acidic composition. In some embodiments, the acidic composition comprises about 0.2% (w/v) SDS and the (NH₄)₂SO₄ is at a concentration of about less than 5 mM. In some embodiments, the acidic composition further comprises MgSO₄.

The one or more surfactants can comprise Triton X-100. The Triton X-100 surfactant can comprise about 0.01% (w/v) to about 1.0% (w/v) of the acidic composition. In some embodiments, the acidic composition comprises about 0.1% Triton X-100. In some embodiments, the acidic composition comprises about 0.1% Triton X-100 and less than about 5 mM ammonium sulfate ((NH₄)₂SO₄) and/or magnesium sulfate (MgSO₄). In some embodiments, the acidic composition comprises about 0.1% Triton X-100, about 5 mM ammonium sulfate ((NH₄)₂SO₄) and about 4 mM magnesium sulfate (MgSO₄).

### Chelators and Reducing Agents

In some embodiments, the acidic composition comprises a chelating agent, a reducing agent, or both. In some embodiments, the acidic composition does not comprise a reducing agent, a chelating agent, or both. The chelating agent can be ethylene diamine tetra-acetate (EDTA), ethylene glycol bis (amino ethyl) N,N′-tetra-acetate (EGTA), nitrilo-tri-acetate (NTA), Tris, or a combination thereof. Examples of divalent ions (e.g., magnesium ions)-chelating agents include, but are not limited to, 1,2-bis(2-aminophenoxy) ethane-N,N,N′,N′-tetraacetic acid, EDTA, ethylene glycol-bis (2-aminoethylether)-N,N,N′,N′-tetraacetic acid, EGTA, 1,2-bis (o-aminophenoxy) ethane-N,N,N′,N′-tetraacetic acid (BAPTA), a buffer containing citrate, N,N-Bis(2-(bis-(carboxymethyl) amino) ethyl)-glycine (DTPA), NTA, a buffer that precipitates a calcium ion from the sample (e.g., a phosphate buffer, a carbonate buffer and a bicarbonate buffer), citric acids and its salts, gluconic acid and its salts, alkali metal pyrophosphates, alkali metal polyphosphates, sodium hexametaphosphate, triethylene tetramine, diethylene triamine, o-phenanthroline, oxalic acid, or any combination thereof. The reducing agent can be 2-mercaptoethanol, dithiothreitol (DTT), tris (2-carboxyethyl) phosphine (TCEP), dithioerythritol (DTE), reduced glutathione, cysteamine, tri-n-butylphosphine (TBP), dithioerythriol, tris (3-hydroxypropyl) phosphine (THPP), 2-mercaptoethylamin-HCl, dithiobutylamine (DTBA), cysteine, cysteine-thioglycolate, salts of sulfurous acid, thioglycolic acid and hydroxyethyldisulphide (HED), or any combination thereof.

The chelating agent and/or the reducing agent can be present in the acidic composition at a concentration in the range of about 0.1 mM to about 14 mM, for example about 0.1, 0.2, 0.3, 0.4, 0.5, 0.6, 0.7, 0.8, 0.9, 1, 2, 3, 4, 5, 6, 7, 8, 9, 10, 11, 12, 13, or 14 mM, or a number or a range between any two of these values.

### Reagent Composition

The reagent compositions described herein (e.g., dried composition) can be provided in a “dry form,” or in a form not suspended in liquid medium. The “dry form” of the compositions can include dry powders, lyophilized compositions, spray-dried, or precipitated compositions. The “dry form” compositions can include one or more lyoprotectants, such as sugars and their corresponding sugar alcohols, such as sucrose, lactose, trehalose, dextran, erythritol, arabitol, xylitol, sorbitol, and mannitol; amino acids, such as arginine or histidine; lyotropic salts, such as MgSO₄; polyols, such as propylene glycol, glycerol, poly (ethylene glycol), or polypropylene glycol); and combinations thereof. Additional exemplary lyoprotectants include gelatin, dextrins, modified starch, and carboxymethyl cellulose. As used herein, the terms “lyophilization”, “lyophilized”, and “freeze-dried” refer to a process by which the material to be dried is first frozen and then the ice or frozen solvent is removed by sublimation in a vacuum environment. “Lyophilisate” refers to a lyophilized substance. As disclosed herein, the dried composition can comprise one or more additives and one or more amplification reagents. The compositions described herein (e.g., wet composition) can be provided in a “wet form,” or in a form suspended in liquid medium.

The dried composition can be frozen or lyophilized or spray dried. The dried composition can be heat dried. The dried composition can comprise one or more additives (e.g., a polymer, a sugar or sugar alcohol). The sugar or sugar alcohol can comprise sucrose, lactose, trehalose, dextran, erythritol, arabitol, xylitol, sorbitol, mannitol, or any combination thereof. The polymer can comprise polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof. The one or more additives can comprise one or more amino acids. The one or more additives can comprise Tween 80, Tween 20 and/or Triton X-100. In some embodiments, the one or more additives help lyophilization of the reaction compositions and/or the dissolution of dried pellets. The one or more additives can comprise a nonionic detergent at a concentration of about 0.01% in the dried composition (e.g., dried pellet).

The frozen or lyophilized or spray dried or heat dried composition or the aqueous composition for preparing the frozen or lyophilized or spray dried composition can comprise one or more of the following: (i) Non-aqueous solvents such as ethylene glycol, glycerol, dimethylsulphoxide, and dimethylformamide. (ii) Surfactants such as Tween 80, Brij 35, Brij 30, Lubrol-px, Triton X-10; Pluronic F127 (polyoxyethylene-polyoxypropylene copolymer) also known as poloxamer, poloxamine, and SDS. (iii) Disaccharides such as trehalose, sucrose, lactose, and maltose. (iv) Polymers (which can have different MWs) such as polyethylene glycol, dextran, polyvinyl alcohol), hydroxypropyl methylcellulose, gelatin, polyvinylpyrrolidone, hydroxyethyl cellulose, Ficoll, and albumin. (v) Amino acids such as glycine, proline, 4-hydroxyproline, L-serine, glutamate, alanine, lysine, sarcosine, and gamma-aminobutyric acid.

The reagent composition (e.g., a dried composition, a wet composition) can comprise one or more amplification reagents. In some embodiments, the reagent composition can comprise a reducing agent, a chelating agent, or both. In some embodiments, the reagent composition does not comprise a reducing agent, a chelating agent, or both. The chelating agent can be EDTA, EGTA, NTA, Tris, or any combination thereof. The reducing agent can be DTT, TCEP, DTE, reduced glutathione, cysteamine, TBP, dithioerythriol, THPP, 2-mercaptoethylamin-HCl, DTBA, cysteine, cysteine-thioglycolate, salts of sulfurous acid, thioglycolic acid, HED, or any combination thereof.

### Kits

Provided herein are kits for isothermal amplification and detection of genomic DNA in a biological sample. In some embodiments, the kit contains an acidic composition (e.g., elution/lysis solution) comprising lytic reagents, magnesium, and either an acid or, e.g., a low pH buffer such as glycine/HCl at pH 2.2. In some embodiments, the kit also comprises reagent reaction components comprising primers, enzymes, dNTPs, detection probes, and a buffer with sufficient capacity to maintain optimal pH for amplification after mixing with the acidic composition (e.g., elution/lysis solution).

Disclosed herein include kits for detecting a target nucleic acid sequence in a sample. In some embodiments, the kit comprises: (a) an acidic composition as provided herein, wherein the acidic composition is capable of lysing biological entities to release sample nucleic acids comprised therein, wherein the sample nucleic acids comprise dsDNA suspected of comprising a target nucleic acid sequence, wherein the target nucleic acid sequence is no longer than 100 nucleotides in length. In some embodiments, the kit comprises: (b) a reagent composition (e.g., a dried composition, a wet composition) comprising a buffering agent and one or more amplification reagents for amplifying the target nucleic acid sequence under isothermal amplification conditions, wherein said one or more amplification reagents comprise: (i) a first primer and a second primer, wherein the first primer is capable of hybridizing to a sequence of a first strand of the target nucleic acid sequence, and the second primer is capable to hybridizing to a sequence of a second strand of the target nucleic acid sequence; and (ii) an enzyme having a hyperthermophile polymerase activity capable of generating a nucleic acid amplification product.

The buffering agent can be MES, MOPS, MOPSO, Tris, HEPES, phosphate, acetate, citrate, succinate, ammonium buffers, or any combination thereof. The kit can comprise: at least one component providing real-time detection activity for a nucleic acid amplification product. The real-time detection activity can be provided by a molecular beacon. A kit can include a control polynucleotide, and where multiple target sequences are amplified, a plurality of control polynucleotides can be included in the kit. In some embodiments, the enzyme having a hyperthermophile polymerase activity has an amino acid sequence that is at least about 90% or 95% identical to the amino acid sequence of SEQ ID NO: 1 or a functional fragment thereof. The enzyme having a hyperthermophile polymerase activity can be a polymerase comprising the amino acid sequence of SEQ ID NO: 1.

The nucleic acid amplification product can be about 20 to 40 bases long. The nucleic acid amplification product can comprise: (1) the sequence of the first primer, and the reverse complement thereof, (2) the sequence of the second primer, and the reverse complement thereof, and (3) a spacer sequence flanked by (1) the sequence of the first primer and the reverse complement thereof and (2) the sequence of the second primer and the reverse complement thereof, wherein the spacer sequence is 1 to 10 bases long.

The biological entities can comprise one or more of prokaryotic cells, eukaryotic cells, viral particles, exosomes, protoplasts, and microvesicles. The biological entities can comprise a virus, a bacteria, a fungi, a protozoa, portions thereof, or any combination thereof. The target nucleic acid sequence can be a nucleic acid sequence of a virus, bacteria, fungi, or protozoa. The sample nucleic acids can be derived from a virus, bacteria, fungi, or protozoa as disclosed herein.

The first primer and/or the second primer can be about 8 to 16 bases long. The first primer and/or the second primer can comprise one or more of DNA bases, modified DNA bases, or a combination thereof.

The reagent composition (e.g., a dried composition) can be lyophilized and/or heat-dried and can comprise one or more additives. The one or more additives comprise: an amino acid; a sugar or sugar alcohol. The sugar or sugar alcohol can comprise sucrose, lactose, trehalose, dextran, erythritol, arabitol, xylitol, sorbitol, mannitol, or any combination thereof. The one or more additives can comprise a polymer. The polymer can comprise polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof.

A mixture of the acidic composition and the reagent composition can have a pH of about 7 to about 9, e.g., a pH of about 8.8. In some embodiments, a mixture of the sample, the acidic composition, and the reagent composition has a pH of about 7 to about 9, e.g., a pH of about 8.8. The buffering agent can comprise Tris. A mixture of the sample, the acidic composition, and the reagent composition can comprise Tris at a concentration in the range of about 30 mM Tris to about 50 mM Tris. The kit can comprise a sterile container housing the acidic composition and the reagent composition.

Kits can also comprise one or more of the components in any number of separate vessels, chambers, containers, packets, tubes, vials, microtiter plates and the like, or the components can be combined in various combinations in such containers. Components of the kit can, for example, be present in one or more containers. In some embodiments, all of the components are provided in one container. In some embodiments, the enzymes (e.g., polymerase(s) and/or reverse transcriptase(s)) can be provided in a separate container from the primers. The components can, for example, be lyophilized, heat dried, freeze dried, or in a stable buffer. In some embodiments, polymerase(s) and/or reverse transcriptase(s) are in lyophilized form or heat dried form in a single container, and the primers are either lyophilized, heat dried, freeze dried, or in buffer, in a different container. In some embodiments, polymerase(s) and/or reverse transcriptase(s), and the primers are, in lyophilized form or heat dried form, in a single container.

Kits can further comprise dNTPs used in the reaction, or modified nucleotides, vessels, cuvettes or other containers used for the reaction, or a vial of water or buffer for re-hydrating lyophilized or heat-dried components. The buffer used can, for example, be appropriate for both polymerase and primer annealing activity.

Kits can also comprise instructions for performing one or more methods described herein and/or a description of one or more components described herein. Instructions and/or descriptions can be in printed form and can be included in a kit insert. A kit also can include a written description of an internet location that provides such instructions or descriptions.

Kits can further comprise reagents used for detection methods, for example, reagents used for FRET, lateral flow devices, dipsticks, fluorescent dye, colloidal gold particles, latex particles, a molecular beacon, or polystyrene beads.

### EXAMPLES

Some aspects of the embodiments discussed above are disclosed in further detail in the following examples, which are not in any way intended to limit the scope of the present disclosure.

### Example 1

### Study of DNA Dissociation in Acidic Solutions

This example demonstrates dsDNA separation in acidic compositions provided herein for use with the amplification and detection methods disclosed herein.

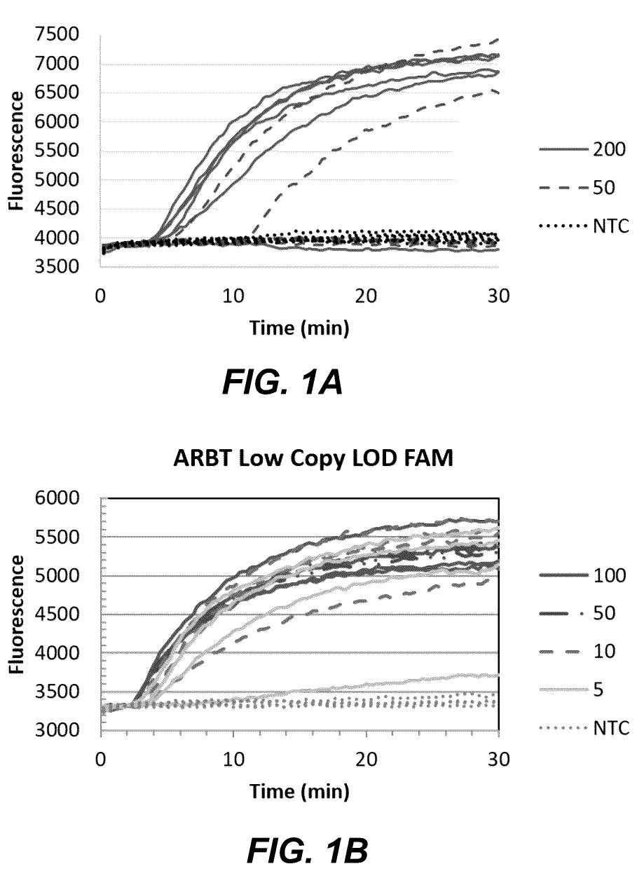

The use of a low pH solution for dissociation of gDNA for APA amplification was investigated. FIG. 1A shows the result of an experiment in which target genomic DNA (gDNA) was pre-incubated in TE solution (10 mM Tris, pH 8.0, 0.1 mM EDTA) at 78° C. for 2 minutes, then cooled down and added to the master mix. In the absence of an acidic component, only 5/6 curves showed amplification at a target input of 200 copies (cps)/reaction, and only 2/6 curves had amplification for 50 copies per reaction. FIG. 1B shows the result of an experiment in which target gDNA was pre-incubated in an acidic solution (5 mM (NH₄)₂SO₄, 4 mM MgSO₄, 0.1% Tx-100, and 10 mM HCl) at 78° C. for 2 minutes, then cooled back down and added to the master mix. This method was found to yield good detection down to 5 copies.

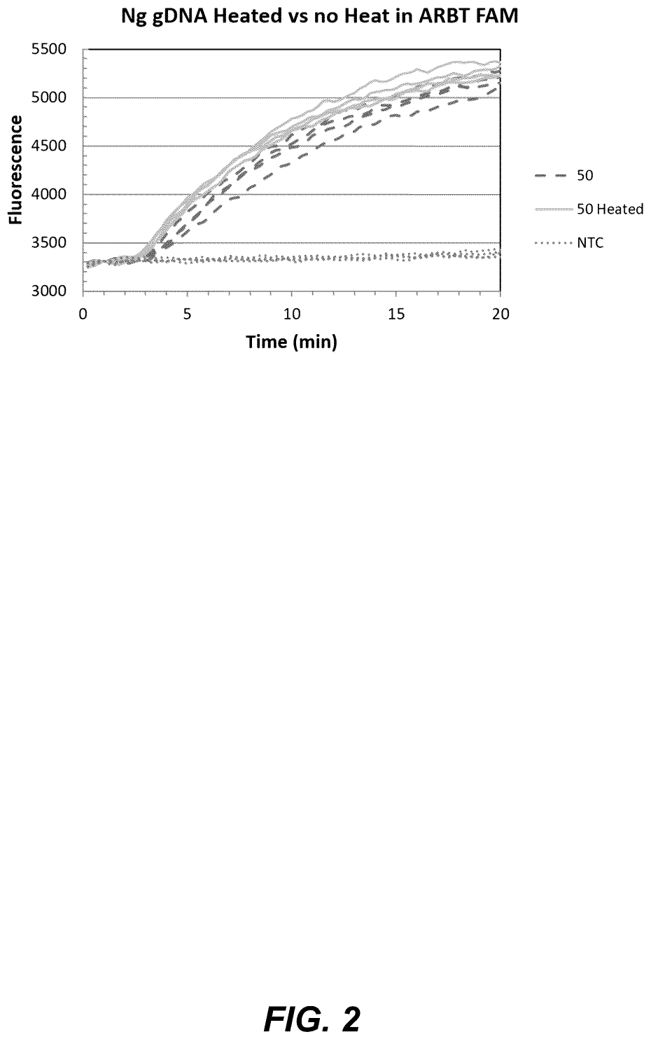

The effect of pre-incubation temperature on N. gonorrhoeae (Ng) gDNA detection was investigated (FIG. 2). The test compared heating vs. no heating for incubating 50 copies of Ng gDNA in acidic solution (5 mM (NH₄)₂SO₄, 4 mM MgSO₄, 0.1% Triton X-100, and 10 mM HCl) for 2 minutes before amplification. The results indicate that initial pre-heating is not necessary for DNA dissociation in an acidic solution. In fact, just pre-incubating the gDNA at room temperature was found to be enough to see the effect.

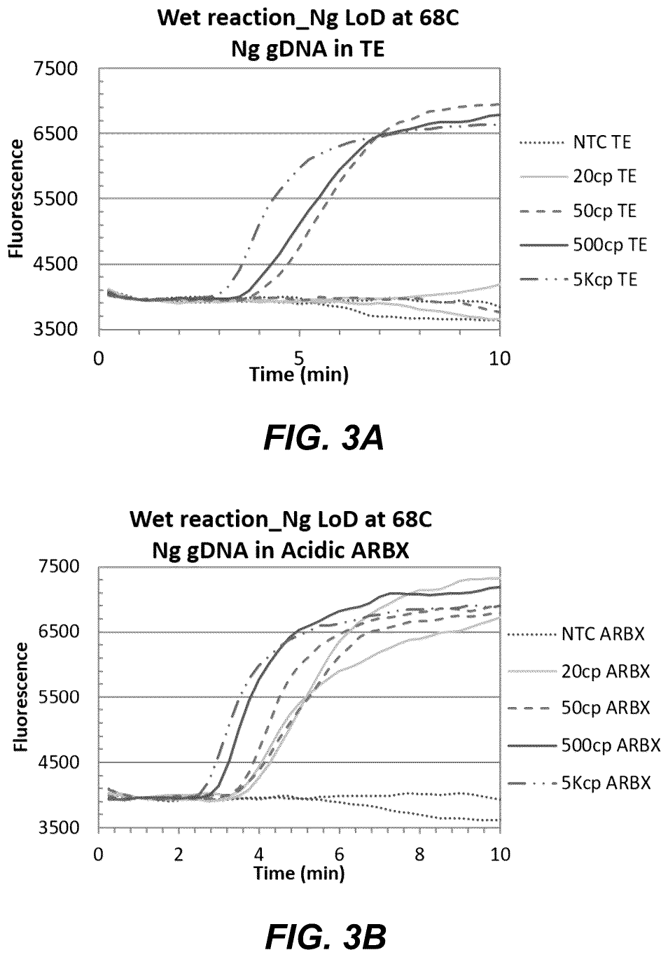

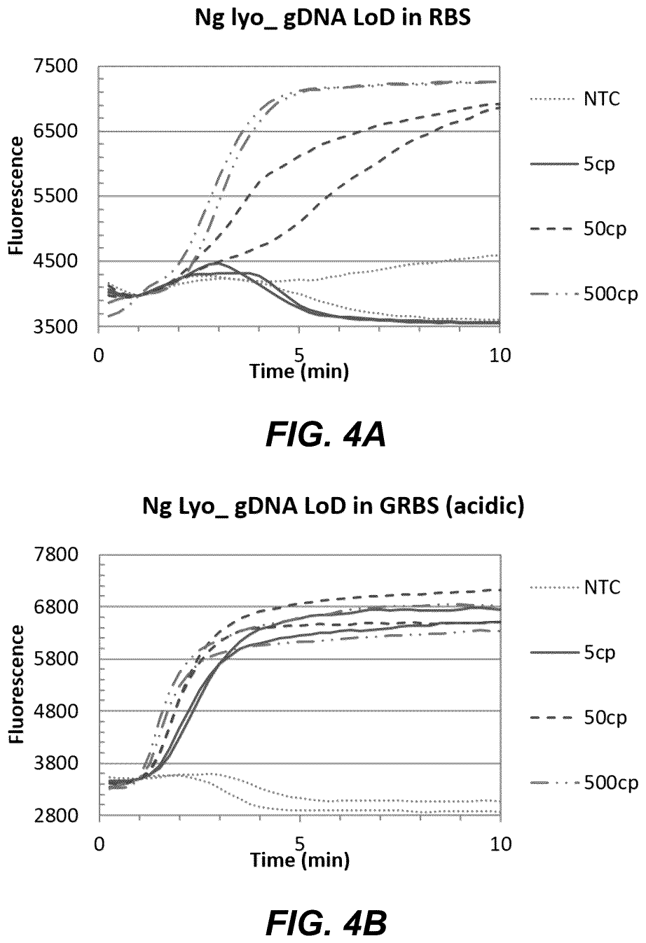

Next, the effect of elution buffer pH on N. gonorrhoeae (Ng) gDNA detection in a wet reaction (FIGS. 3A-3B) and as well as prior to mixing with dried reaction components (FIGS. 4A-4B) was investigated. N. gonorrhoeae (Ng) gDNA was diluted in either an acidic solution (5 mM (NH₄)₂SO₄, 4 mM MgSO₄, 0.1% Tx-100, and 10 mM HCl; FIG. 3B) or a control solution (TE; pH 8; 10 mM Tris, pH 8.0, 0.1 mM EDTA; FIG. 3A) before Archaeal Polymerase Amplification (APA) in wet reaction at 68° C. for 10 minutes. N. gonorrhoeae (Ng) gDNA was diluted in neutral pH elution buffer (5 mM (NH₄)₂SO₄, 4 mM MgSO₄, and 0.2% SDS; FIG. 4A) or acidic elution buffer (5 mM (NH₄)₂SO₄, 4 mM MgSO₄, 10 mM glycine/8.8 mM HCl (glycine/HCl buffer), and 0.2% SDS; FIG. 4B) at room temperature before mixing with dried reaction components containing Tris buffer at pH 8.8 for amplification at 68° C. for 10 minutes.

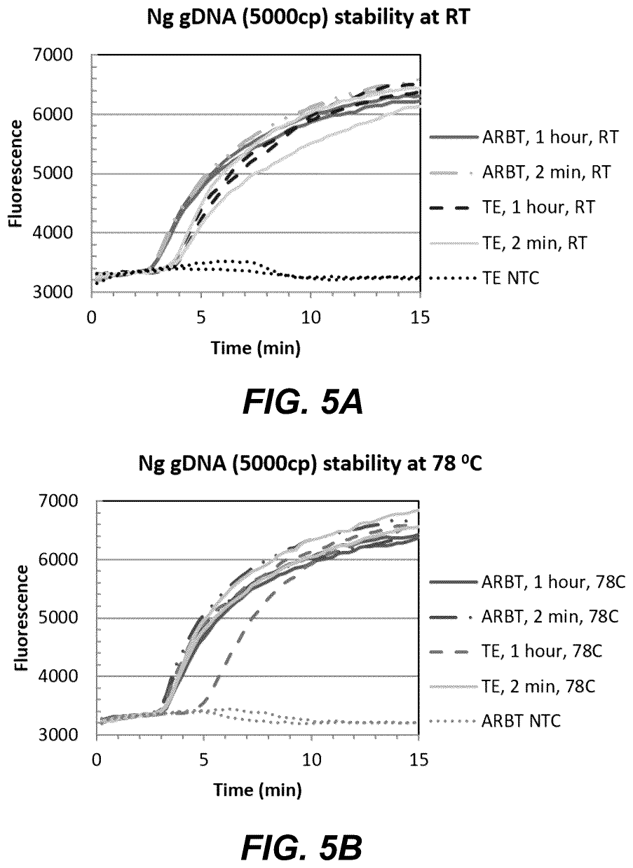

The effect of temperature and pH on N. gonorrhoeae (Ng) gDNA stability was also investigated. 5000 cps of Ng gDNA per reaction were incubated in an acidic solution (5 mM (NH₄)₂SO₄, 4 mM MgSO₄, 0.1% Tx-100, and 10 mM HCl) or TE solution (pH 8; 10 mM Tris, pH 8.0, 0.1 mM EDTA) for 2 minutes vs. 1 hour at 78° C. (FIG. 5B) or room temperature (FIG. 5A) before mixing with amplification components containing Tris buffer at pH 8.8 for amplification at 68° C. for 15 minutes.

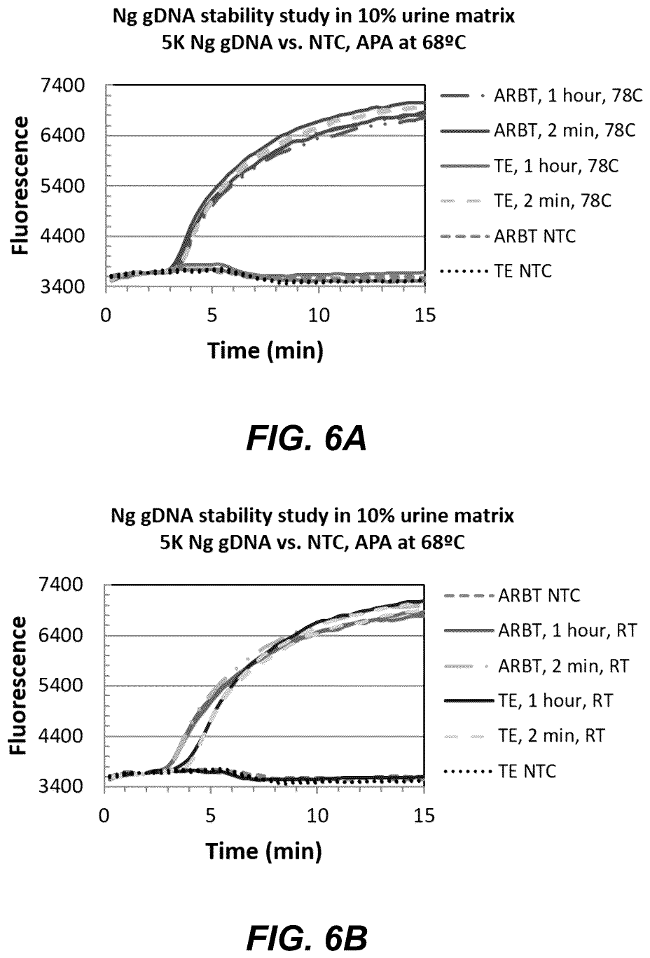

Next the effect of temperature and pH on N. gonorrhoeae (Ng) gDNA stability in 10% urine matrix was investigated. 5000 copies of Ng gDNA per reaction were incubated in an acidic solution (5 mM (NH₄)₂SO₄, 4 mM MgSO₄, 0.1% Tx-100, and 8.8 mM HCl) or TE solution (10 mM Tris, pH 8.0, 0.1 mM EDTA) containing 10% urine for 2 minutes or 1 hour at 78° C. (FIG. 6A) or room temperature (RT; FIG. 6B) before mixing with amplification components containing Tris Buffer (pH 8.8) for amplification at 68° C. for 15 minutes.

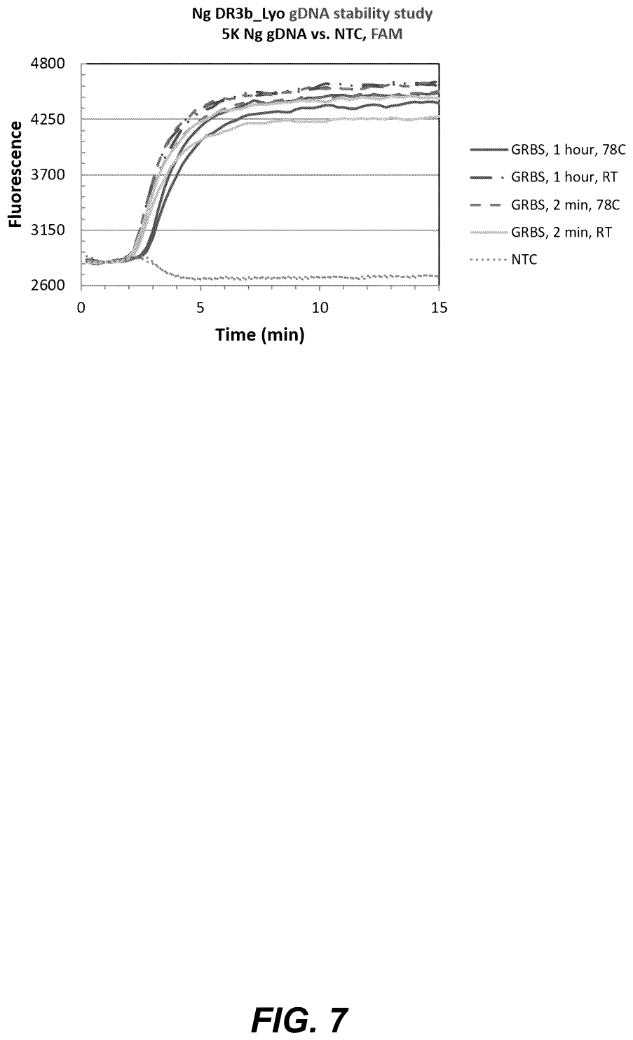

The effect of temperature and pH on N. gonorrhoeae (Ng) gDNA stability in a dry reaction format was also investigated (FIG. 7). 5000 copies of Ng gDNA per reaction were incubated in an acidic solution (5 mM (NH₄)₂SO₄, 4 mM MgSO₄, 10 mM glycine/8.8 mM HCl (glycine/HCl buffer), and 0.2% SDS) for 2 minutes vs. 1 hour at 78° C. or room temperature before mixing with dried reaction components for amplification at 68° C. for 15 minutes. The lyophilized (lyo) pellets contained Tris buffer at pH 8.8.

Collectively, these results yield the surprising finding that exposing genomic DNA to a low pH solution as an initial step in Archaeal Polymerase Amplification does not adversely affect amplification as is commonly believed. Accordingly, the methods, compositions, and kits provided herein can advantageously provide a point-of-care molecular diagnostic for DNA pathogens in clinical samples. Following pathogen lysis, genomic DNA strands can be separated with no need for a separate thermal or enzymatic step.

### Example 2

### Improvements in Assay LODs for gDNA Detection

This example provides approaches for improving the limit of detection (LOD) of assays for detection of gDNA using the disclosed methods and compositions.

In some embodiments, the current limit of detection (LOD) in a dry-down format is >50 copies of gDNA for C. trachomatis/N. gonorrhoeae (Ct/Ng) lyophilized with standard dNTPs. Without being bound by any particular theory, achieving low LOD can comprise destabilizing gDNA in resuspension buffer, and embodiments can involve limiting the effects of Mg²⁺ and monovalent salt, as well as increasing pH to >9.5, or decreasing pH to <4. Some embodiments of the methods and compositions provided herein can overcome various issues, such as resuspension buffer stability, Mg2+ precipitating if in a high pH solution, and the fact that there can already be low salt in resuspension buffer (e.g., 5 mM (NH₄)₂SO₄, 4 mM MgSO₄, 0.2% SDS (7 mM Na⁺, 7 mM DS⁻)).

Factors identified as affecting DNA duplex stability in some embodiments include: % GC, length of dsDNA, sequence dependence, effects of Magnesium and monovalent cations (e.g., Mg²⁺ dominant over monovalent cations on Tm under typical PCR conditions, 10 mM Mg²⁺ equivalent to 1 M Na⁺, 10 mM Tris equivalent to 5 mM Na⁺), extreme pH, and additives.

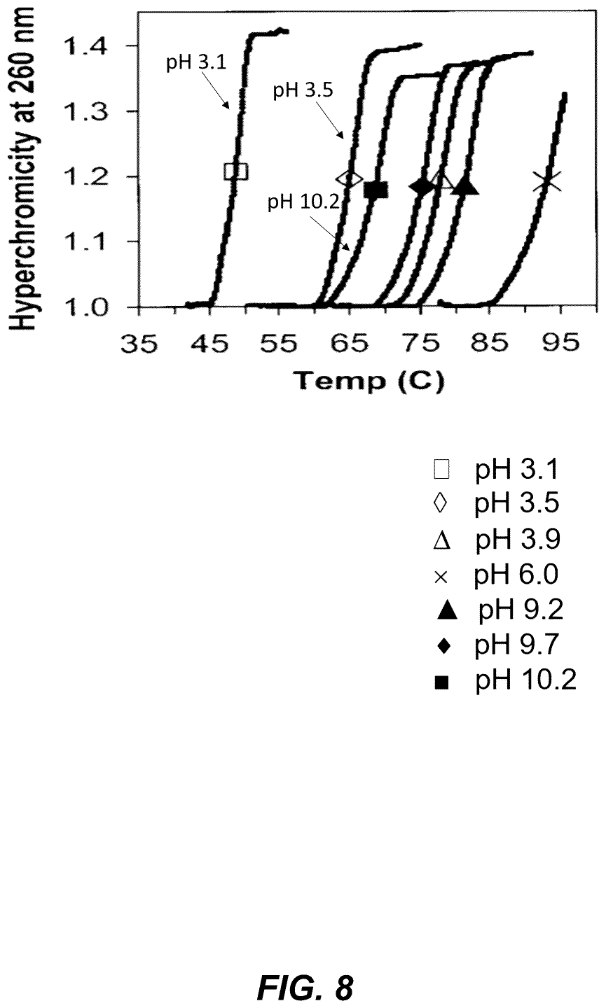

As seen in FIG. 8 (adapted from Biophys J. 2001 February; 80 (2): 874-881. Effect of pH on the overstretching transition of double-stranded DNA: evidence of force-induced DNA melting), which shows non-limiting exemplary melting curves of λ DNA (48502 bps) as a function of pH, a significant Tm decrease at extreme pH (>pH 9.7 and <pH 3.9) is observed. In some embodiments provided herein, low pH is more effective than high pH for λ DNA melting. In some embodiments, LOD can be improved by lowering pH in the resuspension buffer, e.g., resuspension buffer RBS (5 mM (NH₄)₂SO₄, 4 mM MgSO₄, 0.2% SDS, pH ˜7) as compared to a low pH RBS (RBS/10 mM HCl, pH 2-3). No apparent precipitation of RBS/10 mM HCl was observed for three days at room temperature. Embodiments of the compositions and methods provided herein can be optimized to address issues that can arise, such as competition between proton and Mg²⁺ (e.g., Mg²⁺ inhibiting DNA dissociation in low pH solutions) as well as the speed and extent of DNA depurination in low pH solution.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. A method for amplifying a target nucleic acid sequence in a sample, comprising:
(a) contacting a sample comprising biological entities with an acidic composition to generate an acidic mixture,
wherein the acidic composition is capable of lysing the biological entities to release sample nucleic acids comprised therein, wherein the sample nucleic acids comprise double-stranded DNA (dsDNA) suspected of comprising a target nucleic acid sequence, wherein the target nucleic acid sequence is no longer than 100 nucleotides in length,
wherein the acidic composition has a pH of less than 4 and comprises (i) a monovalent salt and/or a divalent salt, (ii) one or more surfactants, and (iii) an acidic agent, thereby denaturing the dsDNA to generate single-stranded DNA (ssDNA);
(b) contacting a reagent composition comprising a buffering agent with the acidic mixture to generate a neutral mixture, wherein the neutral mixture comprises the ssDNA, wherein the reagent composition comprises one or more amplification reagents; and
(c) amplifying the target nucleic acid sequence in the neutral mixture, thereby generating a nucleic acid amplification product, optionally the reagent composition is a dried composition or a wet composition.

2. The method of claim 1, wherein amplifying the target nucleic acid sequence comprises generating the nucleic acid amplification product at detectable levels within about 20 minutes.

3. The method of any one of claims 1-2, wherein the acidic composition has a pH of about 1 to about 6.9, optionally a pH of about 1 to about 3.9, and further optionally a pH of about 2.2.

4. The method of any one of claims 1-3, wherein the neutral mixture has a pH of about 7 to about 9, optionally the neutral mixture has a pH of about 8.8.

5. The method of any one of claims 1-4, the method further comprising detecting the target nucleic acid sequence in the sample, wherein detecting the target nucleic acid sequence in the sample comprises:
(d) detecting the nucleic acid amplification product, wherein the detecting is performed in less than about 20 minutes, less than about 15 minutes, less than about 10 minutes, or less than about 5 minutes, from the time the reagent composition is contacted with the acidic mixture.

6. The method of any one of claims 1-5, wherein contacting the reagent composition with the acidic mixture comprises dissolving the reagent composition in the acidic mixture.

7. The method of any one of claims 1-6, wherein the one or more amplification reagents comprise one or more of an enzyme having a hyperthermophile polymerase activity, a first primer, a second primer, and dNTPs.

8. The method of any one of claims 1-7, wherein the reagent composition is lyophilized and/or heat-dried and comprises one or more additives, wherein the one or more additives comprise:
an amino acid;
a sugar or sugar alcohol, optionally the sugar or sugar alcohol comprises sucrose, lactose, trehalose, dextran, erythritol, arabitol, xylitol, sorbitol, mannitol, or any combination thereof; and/or
a polymer, optionally the polymer comprises polyethylene glycol, dextran, polyvinyl alcohol, hydroxypropyl methylcellulose, gelatin, polyvinylpyrrolidone, hydroxyethyl cellulose, Ficoll, albumin, a polypeptide, a collagen peptide, or any combination thereof.

9. The method of any one of claims 1-8, wherein the dsDNA suspected of comprising the target nucleic acid sequence comprises a first strand and a second strand complementary to each other.

10. The method of any one of claims 1-9, wherein amplifying the target nucleic acid sequence comprises:
amplifying the target nucleic acid sequence in an isothermal amplification condition, wherein the amplifying comprises contacting the ssDNA with:
i) a first primer and a second primer, wherein the first primer is capable of hybridizing to a sequence of the first strand of the target nucleic acid sequence, and the second primer is capable of hybridizing to a sequence of the second strand of the target nucleic acid sequence; and
ii) an enzyme having a hyperthermophile polymerase activity, thereby generating a nucleic acid amplification product, wherein the nucleic acid amplification product comprises:
(1) the sequence of the first primer, and the reverse complement thereof,
(2) the sequence of the second primer, and the reverse complement thereof, and
(3) a spacer sequence flanked by (1) the sequence of the first primer and the reverse complement thereof and (2) the sequence of the second primer and the reverse complement thereof, wherein the spacer sequence is 1 to 10 bases long.

11. The method of any one of claims 1-10, wherein the enzyme having a hyperthermophile polymerase activity has an amino acid sequence that is at least about 90% identical to the amino acid sequence of SEQ ID NO: 1 or a functional fragment thereof, optionally the enzyme having a hyperthermophile polymerase activity has an amino acid sequence that is at least about 95% identical to the amino acid sequence of SEQ ID NO: 1, further optionally the enzyme having a hyperthermophile polymerase activity is a polymerase comprising the amino acid sequence of SEQ ID NO: 1.

12. The method of any one of claims 1-11, wherein the enzyme having a hyperthermophile polymerase activity has low or no exonuclease activity.

13. The method of any one of claims 1-12, wherein the first primer and/or the second primer is about 8 to 16 bases long, optionally the first primer and/or the second primer comprises one or more of DNA bases, modified DNA bases, or a combination thereof.

14. The method of any one of claims 1-13, wherein the nucleic acid amplification product is about 20 to 40 bases long.

15. The method of any one of claims 1-14, wherein the spacer sequence comprises a portion of the target nucleic acid sequence, optionally the spacer sequence is 1 to 10 bases long.

16. The method of any one of claims 1-15, wherein the dsDNA comprises genomic DNA (gDNA), plasmid DNA, or both.

17. The method of any one of claims 1-16,
wherein the biological entities comprise one or more of prokaryotic cells, eukaryotic cells, viral particles, exosomes, protoplasts, and microvesicles; and/or
wherein the biological entities comprise a virus, a bacteria, a fungi, a protozoa, portions thereof, or any combination thereof.

18. The method of any one of claims 1-17, wherein the target nucleic acid sequence is a nucleic acid sequence of a virus, bacteria, fungi, or protozoa, optionally the sample nucleic acids are derived from a virus, bacteria, fungi, or protozoa.

19. The method of claim 18,
wherein the virus is SARS-COV-2, Human Immunodeficiency Virus Type 1 (HIV-1), Human T-Cell Lymphotrophic Virus Type 1 (HTLV-1), Hepatitis B Virus (HBV), Hepatitis C Virus (HCV), Herpes Simplex, Herpesvirus 6, Herpesvirus 7, Epstein-Barr Virus, Respiratory Syncytial Virus (RSV), Cytomegalo-virus, Varicella-Zoster Virus, JC Virus, Parvovirus B19, Influenza A, Influenza B, Influenza C, Rotavirus, Human Adenovirus, Rubella Virus, Human Enteroviruses, Genital Human Papillomavirus (HPV), and Hantavirus;
wherein the bacteria comprises one or more of Mycobacteria tuberculosis, Rickettsia rickettsii, Ehrlichia chaffeensis, Borrelia burgdorferi, Yersinia pestis, Treponema pallidum, Chlamydia trachomatis, Chlamydia pneumoniae, Mycoplasma pneumoniae, Mycoplasma sp., Legionella pneumophila, Legionella dumoffii, Mycoplasma fermentans, Ehrlichia sp., Haemophilus influenzae, Neisseria meningitidis, Neisseria gonorrhoeae, Streptococcus pneumonia, S. agalactiae, and Listeria monocytogenes;
wherein the fungi comprises one or more of Cryptococcus neoformans, Pneumocystis carinii, Histoplasma capsulatum, Blastomyces dermatitidis, Coccidioides immitis, and Trichophyton rubrum; and/or
wherein the protozoa comprises one or more of Trypanosoma cruzi, Leishmania sp., Plasmodium, Entamoeba histolytica, Babesia microti, Giardia lamblia, Cyclospora sp., and Eimeria sp.

20. The method of any one of claims 1-19, wherein the sample is a biological sample or an environmental sample,
wherein the environmental sample is, or is obtained from, a food sample, a beverage sample, a paper surface, a fabric surface, a metal surface, a wood surface, a plastic surface, a soil sample, a fresh water sample, a waste water sample, a saline water sample, exposure to atmospheric air or other gas sample, cultures thereof, or any combination thereof; and/or
wherein the biological sample is, or is obtained from, a tissue sample, saliva, blood, plasma, sera, stool, urine, sputum, mucous, lymph, synovial fluid, cerebrospinal fluid, ascites, pleural effusion, seroma, pus, swab of skin or a mucosal membrane surface, cultures thereof, or any combination thereof.

21. The method of any one of claims 1-20, wherein the acidic agent comprises an organic acid, an inorganic acid, or both.

22. The method of any one of claims 1-21, wherein the acidic agent is selected from the group consisting of hydrochloric acid, glycine hydrochloride, sulfuric acid, acetic acid, citric acid, and phosphoric acid.

23. The method of any one of claims 1-22, wherein the acidic agent is present in the acidic composition at a concentration of less than about 100 mM, optionally in the range of about 1 mM to about 100 mM, and further optionally at a concentration of about 10 mM.

24. The method of any one of claims 1-23, wherein the monovalent salt and/or the divalent salt comprises a sodium salt, a potassium salt, a calcium salt, a magnesium salt, or any combination thereof.

25. The method of any one of claims 1-24, wherein the monovalent salt is selected from the group consisting of ammonium sulfate, sodium fluoride, sodium chloride, sodium bromide, sodium iodide, potassium fluoride, potassium chloride, potassium bromide, and potassium iodide.

26. The method of any one of claims 1-25, wherein the divalent salt is selected from the group consisting of magnesium sulfate, calcium chloride, magnesium chloride, copper (II) chloride, zinc chloride, calcium oxide, magnesium oxide, barium oxide, sodium sulfate, calcium sulfate, copper (II) sulfate, potassium carbonate, and sodium carbonate.

27. The method of any one of claims 1-26, wherein the monovalent salt and/or the divalent salt is present in the acidic composition at a concentration in the range of about 1 mM to about 14 mM, and optionally at a concentration of about 5 mM or about 4 mM.

28. The method of any one of claims 1-27, wherein the one or more surfactants comprises one or more of a cationic surfactant, an anionic surfactant, a non-ionic surfactant, and an amphoteric surfactant.

29. The method of any one of claims 1-28, wherein the one or more surfactants is present in the acidic composition at a concentration in the range of about 0.01% to about 2% weight by volume (% w/v) of the acidic composition, optionally the one or more surfactants is present in the acidic composition at a concentration of about 0.1% weight by volume (% w/v) of the acidic composition.

30. The method of any one of claims 1-29, wherein the acidic composition further comprises a chelating agent, optionally the chelating agent is selected from the group consisting of ethylene diamine tetra-acetate (EDTA), ethylene glycol bis (amino ethyl) N,N′-tetra-acetate (EGTA), nitrilo-tri-acetate (NTA), and Tris.

31. The method of claim 30, wherein the chelating agent is present in the acidic composition at a concentration in the range of about 0.1 mM to about 14 mM.

32. The method of any one of claims 1-31, wherein contacting the sample with the acidic composition is performed at a temperature in the range of about 18° C. to about 99° C., optionally contacting the sample with the acidic composition is performed at a temperature of about 78° C.

33. The method of any one of claims 1-31, wherein contacting the sample with the acidic composition is performed at a temperature in the range of about 18° C. to about 25° C.

34. The method of any one of claims 1-33, wherein contacting the sample with the acidic composition is performed for a period of about 5 seconds to about 60 minutes.

35. The method of any one of claims 1-34, wherein the buffering agent comprises MES, MOPS, MOPSO, Tris, HEPES, phosphate, acetate, citrate, succinate, ammonium buffers, or any combination thereof.

36. The method of any one of claims 1-35, wherein the neutral mixture has a pH in the range of about 7 to about 9, optionally the neutral mixture has a pH of about 8.8.

37. The method of any one of claims 1-36, wherein the target nucleic acid sequence comprises a length of no longer than about 90 nucleotides, optionally the target nucleic acid sequence comprises a length of about 30 nucleotides.

38. The method of any one of claims 1-37, wherein the amplifying is performed in an isothermal amplification condition, optionally the isothermal amplification condition comprises a constant temperature of about 30° C. to about 72° C., further optionally the isothermal amplification condition comprises a constant temperature of about 67° C.

39. The method of any one of claims 1-38, wherein the amplifying is performed for a period of about 5 minutes, about 6 minutes, about 7 minutes, about 8 minutes, about 9 minutes, about 10 minutes, about 20 minutes, about 30 minutes, about 40 minutes, about 50 minutes, or about 60 minutes, optionally the amplifying is performed for a period of about 15 minutes.

40. The method of any one of claims 1-39, wherein the amplifying is performed in helicase-free, single-stranded binding protein-free, cleavage agent-free, and recombinase-free, isothermal amplification conditions.

41. The method of any one of claims 1-40, wherein step (d) further comprises determining the amount of the dsDNA that comprises the target nucleic acid sequence in the sample.

42. The method of any one of claims 1-41, wherein detecting the nucleic acid amplification product comprises use of a real-time detection method.

43. The method of any one of claims 1-42, wherein detecting the nucleic acid amplification product comprises contacting the nucleic acid amplification product with a signal-generating oligonucleotide capable of hybridizing to the nucleic acid amplification product, wherein the single-generating oligonucleotide comprises a fluorophore, a quencher, or both.

44. The method of any one of claims 1-43, wherein detecting the nucleic acid amplification product comprises detecting a fluorescent signal, optionally the fluorescent signal is from a molecular beacon.

45. The method of any one of claims 1-44, wherein the method is performed in a single reaction vessel.

46. The method of any one of claims 1-45, wherein the amplifying comprises multiplex amplification of two or more target nucleic acid sequences, and wherein the detecting comprises multiplex detection of two or more nucleic acid amplification products derived from said two or more target nucleic acid sequences, optionally the two or more target nucleic acid sequences are specific to two or more different organisms.

47. The method of any one of claims 1-46, wherein the amplifying does not comprise using any enzyme other than the enzyme having a hyperthermophile polymerase activity.

48. The method of any one of claims 1-47, wherein the amplifying comprises one or more of the following: Archaeal Polymerase Amplification (APA), loop-mediated isothermal Amplification (LAMP), helicase-dependent Amplification (HDA), recombinase polymerase amplification (RPA), strand displacement amplification (SDA), nucleic acid sequence-based amplification (NASBA), transcription mediated amplification (TMA), nicking enzyme amplification reaction (NEAR), rolling circle amplification (RCA), multiple displacement amplification (MDA), Ramification (RAM), circular helicase-dependent amplification (cHDA), single primer isothermal amplification (SPIA), signal mediated amplification of RNA technology (SMART), self-sustained sequence replication (3SR), genome exponential amplification reaction (GEAR) and isothermal multiple displacement amplification (IMDA).

49. The method of any one of claims 1-48, wherein the amplifying does not comprise one or more of the following: Archaeal Polymerase Amplification (APA), LAMP, HDA, RPA, SDA, NASBA, TMA, NEAR, RCA, MDA, RAM, cHDA, SPIA, SMART, 3SR, GEAR and IMDA, and optionally the amplifying does not comprise LAMP.

50. The method of any one of claims 1-49, wherein the method does not comprise one or more of the following: (i) dilution of the acidic mixture; (ii) dilution of the neutral mixture; (iii) heat denaturation of the acidic mixture; (iv) sonication of the acidic mixture; (v) sonication of the neutral mixture; (vi) the addition of ribonuclease inhibitors to the acidic mixture; (vii) the addition of ribonuclease inhibitors to the neutral mixture; (viii) purification of the sample; (ix) purification of the sample nucleic acids; (x) purification of the nucleic acid amplification product; (xi) removal of the one or more surfactants from the acidic mixture or the neutral mixture; (xii) heating denaturing and/or enzymatic denaturing of the sample nucleic acids prior to and/or during amplification; (xiii) the addition of ribonuclease H to the acidic mixture or the neutral mixture; (xiv) contacting the sample, the acidic mixture, or the neutral mixture with a helicase, a single-stranded binding protein, a nicking enzyme, a restriction enzyme, or a recombinase; (xv) contacting the sample, the acidic mixture, or the neutral mixture with glycerol, formamide, or urea; (xvi) purifying the ssDNA from the acidic mixture or the neutral mixture; (xvii) a heat denaturing step; and (xviii) enriching, purifying and/or isolating the dsDNA.

51. The method of any one of claims 1-50, wherein the acidic composition and/or the reagent composition does not comprise a reducing agent, a chelating agent, or both.

52. The method of claim 51,
wherein the chelating agent is selected from the group comprising ethylene diamine tetra-acetate (EDTA), ethylene glycol bis (amino ethyl) N,N′-tetra-acetate (EGTA), nitrilo-tri-acetate (NTA), Tris, or any combination thereof; and/or
wherein the reducing agent is selected from the group 2-mercaptoethanol, dithiothreitol (DTT), tris (2-carboxyethyl) phosphine (TCEP), dithioerythritol (DTE), reduced glutathione, cysteamine, tri-n-butylphosphine (TBP), dithioerythriol, tris (3-hydroxypropyl) phosphine (THPP), 2-mercaptoethylamine-HCl, dithiobutylamine (DTBA), cysteine, cysteine-thioglycolate, salts of sulfurous acid, thioglycolic acid and hydroxyethyldisulfide (HED), or any combination thereof.

53. The method of any one of claims 1-52, wherein the acidic composition does not comprise both an anionic surfactant and a cationic surfactant.

54. The method of any one of claims 1-53, wherein the acidic composition further comprises a tween surfactant, optionally the tween surfactant is selected from the group consisting of Tween 20, Tween 40, Tween 45, Tween 60, Tween 65, Tween 80, Tween 81 and Tween 85, further optionally the tween surfactant comprises about 0.01% (w/v) of the acidic composition.

55. An acidic composition for lysing biological entities and denaturing double-stranded DNA (dsDNA) comprised therein, comprising:
a monovalent salt at a concentration of less than 30 mM in the acidic composition and/or a divalent salt at a concentration of less than 15 mM in the acidic composition;
one or more surfactants; and
an acidic agent present in the acidic composition at a concentration of less than 100 mM, and
wherein the acidic composition has a pH less than 4.

56. The acidic composition of claim 55, wherein the acidic composition does not comprise a reducing agent, a chelating agent, or both.

57. The acidic composition of claim 56,
wherein the chelating agent is selected from the group comprising ethylene diamine tetra-acetate (EDTA), ethylene glycol bis (amino ethyl) N,N′-tetra-acetate (EGTA), nitrilo-tri-acetate (NTA), Tris, or any combination thereof; and/or
wherein the reducing agent is selected from the group 2-mercaptoethanol, dithiothreitol (DTT), tris (2-carboxyethyl) phosphine (TCEP), dithioerythritol (DTE), reduced glutathione, cysteamine, tri-n-butylphosphine (TBP), dithioerythriol, tris (3-hydroxypropyl) phosphine (THPP), 2-mercaptoethylamine-HCl, dithiobutylamine (DTBA), cysteine, cysteine-thioglycolate, salts of sulfurous acid, thioglycolic acid and hydroxyethyldisulfide (HED), or any combination thereof.

58. The acidic composition of any one of claims 55-57, wherein the acidic composition does not comprise both an anionic surfactant and a cationic surfactant.

59. The acidic composition of any one of claims 55-58, wherein the acidic composition does not comprise glycerol, formamide, or urea.

60. The acidic composition of any one of claims 55-59, wherein the acidic composition has a pH in the range of about 1 to about 3.9, optionally the acidic composition has a pH of about 2.

61. The acidic composition of any one of claims 55-60, wherein the acidic agent comprises an organic acid and/or an inorganic acid.

62. The acidic composition of any one of claims 55-61, wherein the acidic agent is selected from the group consisting of hydrochloric acid, glycine hydrochloride, acetic acid, citric acid, and phosphoric acid.

63. The acidic composition of any one of claims 55-62, wherein the acidic agent is present in the acidic composition at a concentration in the range of about 1 mM to about 100 mM, optionally the acidic agent is present in the acidic composition at a concentration of about 10 mM.

64. The acidic composition of any one of claims 55-63, wherein the monovalent salt and/or the divalent salt comprises a sodium salt, a potassium salt, a calcium salt, a magnesium salt, or any combination thereof.

65. The acidic composition of any one of claims 55-64, wherein the monovalent salt is selected from the group consisting of ammonium sulfate, ammonium chloride, sodium fluoride, sodium chloride, sodium bromide, sodium iodide, potassium fluoride, potassium chloride, potassium bromide, and potassium iodide.

66. The acidic composition of any one of claims 55-65, where the divalent salt is selected from the group consisting of magnesium sulfate, calcium chloride, magnesium chloride, copper (II) chloride, zinc chloride, calcium oxide, magnesium oxide, barium oxide, sodium sulfate, calcium sulfate, copper (II) sulfate, potassium carbonate, and sodium carbonate.

67. The acidic composition of any one of claims 55-66, wherein the monovalent salt and/or the divalent salt is present in the acidic composition at a concentration in the range of about 1 mM to about 14 mM, optionally the monovalent salt and/or the divalent salt is present in the acidic composition at a concentration of about 5 mM or about 4 mM.

68. The acidic composition of any one of claims 55-67, wherein the one or more surfactants comprises one or more of a cationic surfactant, an anionic surfactant, a non-ionic surfactant, and an amphoteric surfactant.

69. The acidic composition of any one of claims 55-68, wherein the one or more surfactants is present in the acidic composition at a concentration in the range of about 0.01% to about 2% weight by volume (% w/v) of the acidic composition, optionally wherein the one or more surfactants is present in the acidic composition at a concentration of about 0.1% weight by volume (% w/v) of the acidic composition.

70. The acidic composition of any one of claims 55-69, wherein the acidic composition further comprises a tween surfactant, optionally the tween surfactant is selected from the group consisting of Tween 20, Tween 40, Tween 45, Tween 60, Tween 65, Tween 80, Tween 81 and Tween 85, further optionally the tween surfactant comprises about 0.01% to about 1% (w/v) of the acidic composition.

71. The acidic composition of any one of claims 55-70, wherein the acidic composition further comprises a chelating agent, optionally the chelating agent is selected from the group comprising ethylene diamine tetra-acetate (EDTA), ethylene glycol bis (amino ethyl) N,N′-tetra-acetate (EGTA), nitrilo-tri-acetate (NTA), Tris, or any combination thereof, further optionally the chelating agent is present in the acidic composition at a concentration in the range of about 0.1 mM to about 14 mM.

72. A kit for detecting a target nucleic acid sequence in a sample, comprising:
(a) the acidic composition of any one of claims 55-71, wherein the acidic composition is capable of lysing biological entities to release sample nucleic acids comprised therein, wherein the sample nucleic acids comprise double-stranded DNA (dsDNA) suspected of comprising a target nucleic acid sequence, wherein the target nucleic acid sequence is no longer than 100 nucleotides in length; and
(b) a reagent composition comprising a buffering agent and one or more amplification reagents for amplifying the target nucleic acid sequence under isothermal amplification conditions, wherein said one or more amplification reagents comprise:
(i) a first primer and a second primer, wherein the first primer is capable of hybridizing to a sequence of a first strand of the target nucleic acid sequence, and the second primer is capable of hybridizing to a sequence of a second strand of the target nucleic acid sequence; and
(ii) an enzyme having a hyperthermophile polymerase activity capable of generating a nucleic acid amplification product,
optionally the reagent composition is a dried composition or a wet composition.

73. The kit of claim 72, wherein the buffering agent is selected from the group comprising MES, MOPS, MOPSO, Tris, HEPES, phosphate, acetate, citrate, succinate, ammonium buffers, or any combination thereof.

74. The kit of any one of claims 72-73, further comprising at least one component providing real-time detection activity for a nucleic acid amplification product, optionally the real-time detection activity is provided by a molecular beacon.

75. The kit of any one of claims 72-74, wherein the enzyme having a hyperthermophile polymerase activity has an amino acid sequence that is at least about 90% identical to the amino acid sequence of SEQ ID NO: 1 or a functional fragment thereof, optionally the enzyme having a hyperthermophile polymerase activity has an amino acid sequence that is at least about 95% identical to the amino acid sequence of SEQ ID NO: 1, further optionally the enzyme having a hyperthermophile polymerase activity is a polymerase comprising the amino acid sequence of SEQ ID NO: 1.

76. The kit of any one of claims 72-75, wherein the nucleic acid amplification product is about 20 to 40 bases long, and wherein the nucleic acid amplification product comprises:
(1) the sequence of the first primer, and the reverse complement thereof,
(2) the sequence of the second primer, and the reverse complement thereof, and
(3) a spacer sequence flanked by (1) the sequence of the first primer and the reverse complement thereof and (2) the sequence of the second primer and the reverse complement thereof, wherein the spacer sequence is 1 to 10 bases long.

77. The kit of any one of claims 72-76, wherein the biological entities comprise one or more of prokaryotic cells, eukaryotic cells, viral particles, exosomes, protoplasts, and microvesicles, optionally the biological entities comprise a virus, a bacteria, a fungi, a protozoa, portions thereof, or any combination thereof.

78. The kit of any one of claims 72-77, wherein the target nucleic acid sequence is a nucleic acid sequence of a virus, bacteria, fungi, or protozoa, optionally the sample nucleic acids are derived from a virus, bacteria, fungi, or protozoa.

79. The kit of claim 78,
wherein the virus is SARS-COV-2, Human Immunodeficiency Virus Type 1 (HIV-1), Human T-Cell Lymphotrophic Virus Type 1 (HTLV-1), Hepatitis B Virus (HBV), Hepatitis C Virus (HCV), Herpes Simplex, Herpesvirus 6, Herpesvirus 7, Epstein-Barr Virus, Respiratory Syncytial Virus (RSV), Cytomegalo-virus, Varicella-Zoster Virus, JC Virus, Parvovirus B19, Influenza A, Influenza B, Influenza C, Rotavirus, Human Adenovirus, Rubella Virus, Human Enteroviruses, Genital Human Papillomavirus (HPV), and Hantavirus;
wherein the bacteria comprises one or more of Mycobacteria tuberculosis, Rickettsia rickettsii, Ehrlichia chaffeensis, Borrelia burgdorferi, Yersinia pestis, Treponema pallidum, Chlamydia trachomatis, Chlamydia pneumoniae, Mycoplasma pneumoniae, Mycoplasma sp., Legionella pneumophila, Legionella dumoffii, Mycoplasma fermentans, Ehrlichia sp., Haemophilus influenzae, Neisseria meningitidis, Neisseria gonorrhoeae, Streptococcus pneumonia, S. agalactiae, and Listeria monocytogenes;
wherein the fungi comprises one or more of Cryptococcus neoformans, Pneumocystis carinii, Histoplasma capsulatum, Blastomyces dermatitidis, Coccidioides immitis, and Trichophyton rubrum; and/or
wherein the protozoa comprises one or more of Trypanosoma cruzi, Leishmania sp., Plasmodium, Entamoeba histolytica, Babesia microti, Giardia lamblia, Cyclospora sp., and Eimeria sp.

80. The kit of any one of claims 72-79, wherein the first primer and/or the second primer is about 8 to 16 bases long, optionally the first primer and/or the second primer comprises one or more of DNA bases, modified DNA bases, or a combination thereof.

81. The kit of any one of claims 72-80, wherein the reagent composition is lyophilized and/or heat-dried and comprises one or more additives, wherein the one or more additives comprise:
an amino acid;
a sugar or sugar alcohol, optionally the sugar or sugar alcohol comprises sucrose, lactose, trehalose, dextran, erythritol, arabitol, xylitol, sorbitol, mannitol, or any combination thereof; and/or
a polymer, optionally the polymer comprises polyethylene glycol, dextran,
polyvinyl alcohol, hydroxypropyl methylcellulose, gelatin, polyvinylpyrrolidone, hydroxyethyl cellulose, Ficoll, albumin, a polypeptide, a collagen peptide, or any combination thereof.

82. The kit of any one of claims 72-81, wherein a mixture of the acidic composition and the reagent composition has a pH of about 7 to about 9, optionally a pH of about 8.8.

83. The kit of any one of claims 72-82, wherein a mixture of the sample, the acidic composition, and the reagent composition has a pH of about 7 to about 9, optionally a pH of about 8.8.

84. The kit of any one of claims 72-83, wherein the buffering agent comprises Tris, and wherein a mixture of the sample, the acidic composition, and the reagent composition comprises Tris at a concentration in the range of about 30 mM Tris to about 50 mM Tris.

85. The kit of any one of claims 72-84, wherein the kit comprises a sterile container housing the acidic composition and the reagent composition.


SEQUENCE LISTING (5 sequences)

SEQ ID NO: 1 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIETLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 2 - protein, 775 aa, synthetic construct
MILDTDYITENGKPVIRVFKKENGEFKIEYDRTFEPYFYALLKDDSAIEDVKKVTAKRHGTVVKVKRAEKVQKKFLGRPIEVWKLYFNHPQDVPAIRDRIRAHPAVVDIYEYDIPFAKRYLIDKGLIPMEGDEELTMLAFDIDTLYHEGEEFGTGPILMISYADGSEARVITWKKIDLPYVDVVSTEKEMIKRFLRVVREKDPDVLITYNGDNFDFAYLKKRCEELGIKFTLGRDGSEPKIQRMGDRFAVEVKGRIHFDLYPVIRRTINLPTYTLEAVYEAVFGKPKEKVYAEEIAQAWESGEGLERVARYSMEDAKVTYELGREFFPMEAQLSRLIGQSLWDVSRSSTGNLVEWFLLRKAYKRNELAPNKPDERELARRRGGYAGGYVKEPERGLWDNIVYLDFRSLYPSIIITHNVSPDTLNREGCKEYDVAPEVGHKFCKDFPGFIPSLLGDLLEERQKIKRKMKATVDPLEKKLLDYRQRAIKILANSFYGYYGYAKARWYCKECAESVTAWGREYIEMVIRELEEKFGFKVLYADTDGLHATIPGADAETVKKKAKEFLKYINPKLPGLLELEYEGFYVRGFFVTKKKYAVIDEEGKITTRGLEIVRRDWSEIAKETQARVLEAILKHGDVEEAVRIVKEVTEKLSKYEVPPEKLVIHEQITRDLRDYKATGPHVAVAKRLAARGVKIRPGTVISYIVLKGSGRIGDRAIPADEFDPTKHRYDAEYYIENQVLPAVERILKAFGYRKEDLRYQKTKQVGLGAWLKVKGKK

SEQ ID NO: 3 - DNA, 12 bp, synthetic construct
  source 1..12: mol_type = other DNA
atgcatgcatgc

SEQ ID NO: 4 - DNA, 12 bp, synthetic construct
  source 1..12: mol_type = other DNA
gcatgcatgcat

SEQ ID NO: 5 - DNA, 18 bp, synthetic construct
  source 1..18: mol_type = other DNA
atgcataaaaaagcatgc